(19) United States
(12) Patent Application Publication
(10) Pub. No.: US 2023/0279095 A1
(43) Pub. Date: Sep. 7, 2023

(54) Methods and Treatment for Adult-Onset Still's Disease and Systemic-Onset Juvenile Idiopathic Arthritis Involving Antibodies to IL-18

(71) Applicant: Avalo Therapeutics, Inc., Rockville, MD (US)

(72) Inventors: Garry A. Neil, Havertown, PA (US); H. Jeffrey Wilkins, Ambler, PA (US)

(73) Assignee: Avalo Therapeutics, Inc., Rockville, MD (US)

(21) Appl. No.: 18/068,215

(22) Filed: Dec. 19, 2022

Related U.S. Application Data
(60) Provisional application No. 63/291,694, filed on Dec. 20, 2021.

(51) Int. Cl. C07K 16/24; A61K 39/395; A61K 45/06; A61P 19/02; A61P 37/06

(52) U.S. Cl. CPC C07K 16/244; A61K 39/3955; A61K 45/06; A61P 19/02; A61P 37/06; A61K 2039/545

(57) ABSTRACT

The present disclosure relates to methods of treating adult-onset Still’s disease (AOSD) or systemic-onset juvenile idiopathic arthritis (SoJIA), optionally associated with elevated IL-18 levels, comprising administering to a human subject diagnosed with AOSD or SoJIA an effective amount of an anti-IL-18 antibody at certain dosages.

### CROSS REFERENCE TO RELATED APPLICATIONS

This application claims the benefit of priority to U.S. Provisional Application No. 63/291,694, filed Dec. 20, 2021, the contents of which are incorporated herein by reference for all purposes.

### SEQUENCE LISTING

The instant application contains a Sequence Listing which has been submitted electronically in XML format and is hereby incorporated by reference in its entirety. Said XML copy, created on Dec. 12, 2022, is named “2022-12-12_01339-0003-00US_ST26” and is 229,819 bytes in size.

### FIELD OF THE INVENTION

The present disclosure relates to methods of treating subjects with adult-onset Still’s disease (AOSD) or systemic-onset juvenile idiopathic arthritis (SoJIA) by administering an anti-interleukin 18 (IL-18) neutralizing antibody.

### BACKGROUND OF THE INVENTION

Adult-onset Still’s disease is an inflammatory disorder characterized by daily fevers, arthritis, and an evanescent rash. It was first described in children by George Still in 1897, but later described in adult patients who had features similar to children with systemic juvenile idiopathic arthritis and did not fulfill criteria for classic rheumatoid arthritis. (Bywaters, EG, Ann Rheum Dis. 30(2), 121-133 (1971)). Adult-onset Still’s disease is a rare disease. A retrospective French study estimated the annual incidence of AOSD to be 0.16 cases per 100,000 people, with an equal distribution between the sexes. (Magadur-Joly G, et al., Ann Rheum Dis. 54(7):587 (1995)). It is estimated that there are approximately 3,500 to 7,000 AOSD patients in the United States. (Gerfaud-Valentin M, et al., Medicine (Baltimore) 93(2):91 (2014)). There is a bimodal age distribution, with one peak between the ages of 15 and 25 years and the second between the ages of 36 and 46 years. (Magadur-Joly G, et al., Ann Rheum Dis. 54(7):587 (1995)). However, patients older than 70 years have also been reported. (Steffe LA & Cooke CL, JAMA 249(15):2062 (1983)).

No targeted therapies for AOSD are currently approved in the US. Treatment is tailored to disease stage, symptoms and presence of end organ damage, and the minimization of corticosteroid adverse effects. Initial therapy is usually with non-steroidal anti-inflammatory drugs (NSAIDs) and corticosteroids. Refractory patients are treated with immunosuppressive agents including methotrexate. Patients who remain refractory may be treated empirically with biologic agents including anti-TNF antibodies (adalimumab), anakinra (anti-IL-1), or tocilizumab (anti-IL-6). Patients must be carefully monitored for the development of macrophage activation syndrome (MAS). While many patients may derive at least partial benefit from this approach, others remain refractory or partially refractory. (Jamilloux, Y., et al., Therapeutics and Clinical Risk Management (2014)). Thus, there is a need for new targeted therapies.

The clinical course of AOSD can be divided into three main patterns: monophasic (or monocyclic), intermittent, and chronic, with approximately one-third of patients falling into each category. (Kontzias, A. & Efthimiou, P., Drugs 68(3):319 (2008); Gerfaud-Valentin M, et al., Medicine (Baltimore) 93(2):91 (2014)). Patients with monophasic or intermittent disease frequently progress to the chronic articular pattern. (Kontzias, A. & Efthimiou, P., Drugs 68(3):319 (2008); Gerfaud-Valentin M, et al., Medicine (Baltimore) 93(2):91 (2014); Fautrel, B., Best Pract. Res. Clin. Rheumatol. 22(5):773 (2008)).

Most active AOSD patients have fever, evanescent rash, arthralgias and arthritis. The most commonly involved joints are the knees, wrists, ankles, elbows, proximal interphalangeal joints, and shoulders. (Elkon, KB, et al., Arthritis Rheum. 25(6):647 (1982)). Many patients also have myalgia (Pouchot, J, et al., Medicine (Baltimore) 70(2):118 (1991)), pharyngitis (Nguyen, KH & Weisman MH, J Rheumatol. 24(3):592 (1997)), and liver disease (Gerfaud-Valentin, M, et al., Autoimmun Rev. Jul;13(7):708-722 (2014)). Fulminant hepatic failure has also been reported in AOSD. (Dino, O, et al., J Rheumatol. 23(4):784-785 (1996)). Cardiopulmonary disease (Gerfaud-Valentin, M, et al., Autoimmun Rev. Jul;13(7):708-722 (2014); Cheema, GS & Quismorio, FP Jr. Curr Opin Pulm Med. 5(5):305 (1999)), lymphadenopathy (Gerfaud-Valentin, M, et al., Autoimmun Rev. Jul;13(7):708-722 (2014)), and splenomegaly (Gerfaud-Valentin, M, et al., Autoimmun Rev. Jul;13(7):708-722 (2014)) can also be present.

Adult-onset Still’s disease can be complicated by macrophage activation syndrome (MAS), which is also referred to as hemophagocytic lymphohistiocytosis (HLH) in 12% to 19% of patients. (Arlet, JB, et al., Ann Rheum Dis. 65(12):1596. (2006); Bae, CB, et al., Medicine (Baltimore) 94(4):e451 (2015); Hot, A, et al., Medicine (Baltimore) 89(1):37 (2010)). Other hematological disorders that can be associated with AOSD include microangiopathic hemolytic anemia associated with thrombotic thrombocytopenic purpura-hemolytic uremic syndrome. (Diamond, JR, G. J Nephrol. 10(5):253 (1997); Perez, MG & Rodwig, FR Jr., South Med J. 96(1):46 (2003); Arlet, JB, et al., Ann Rheum Dis. 65(12):1596. (2006)). Abdominal pain may occur in up to half of patients with AOSD (Gerfaud-Valentin, M, et al., Autoimmun Rev. Jul;13(7):708-722 (2014)) and symptoms may be related to lymphadenitis, aseptic peritonitis, or acute pancreatitis (Fautrel, B., Best Pract Res Clin Rheumatol. 22(5):773 (2008)). Adult-onset Still’s disease is diagnosed if ≥ 5 diagnostic criteria are present with ≥ 2 being major criteria and no exclusion criteria. (Jamilloux, Y, et al., Therapeutics and Clinical Risk Management Dec. (2014)). The major diagnostic criteria are: fever > 39° C. lasting > 2 weeks; arthralgia or arthritis lasting > 2 weeks; typical nonpruritic salmon-colored rush; leukocytosis >10,000/mm³; and granulocytes >80%. (Jamilloux, Y, et al., Therapeutics and Clinical Risk Management Dec. (2014)). The minor diagnostic criteria are: sore throat; lymphadenopathy; splenomegaly; abnormal liver function tests; and negative tests for antinuclear antibody and rheumatoid factor. (Jamilloux, Y, et al., Therapeutics and Clinical Risk Management Dec. (2014)). The exclusion criteria are: infection; malignancy; and other rheumatic disease (vasculitis). (Jamilloux, Y, et al., Therapeutics and Clinical Risk Management Dec. (2014)).

Some of the treatment goals of AOSD are: control of the physical signs and symptoms of inflammation (fever, rash, morning stiffness, joint pain, and swelling); control of laboratory indices of inflammation; prevention of end organ damage, including joint injury and other major organ complications; and minimization of the risk of adverse effects of therapy, including short and long-term adverse effects of glucocorticoids.

Systemic-onset juvenile idiopathic arthritis (SoJIA) is similar to AOSD, but differs in terms of classification criteria. (Silva, JR, and Brito, I, Acta Reumatol Port. 45(2):150-151. (2020)). It has been argued that SoJIA and AOSD are the same disease, just happening in different age groups. (Silva, JR, and Brito, I, Acta Reumatol Port. 45(2):150-151. (2020)). SoJIA is a subset of juvenile idiopathic arthritis marked by more severe extra-articular manifestations (fever, cutaneous eruptions). See NIH Genetic and Rare Diseases Information Center, Systemic onset juvenile idiopathic arthritis (available at https://rarediseases.info.nih.gov/diseases/10966/systemic-onset-juvenile-idiopathic-arthritis) (last accessed on Dec. 14, 2021). It represents 10-11% of cases of juvenile idiopathic arthritis. Id. Onset usually occurs between 3 and 5 years of age. Id. The clinical signs include fever with oscillating temperatures over a 24-hour period and peaks of over 39° C. or more, which are associated with transient cutaneous eruptions and diffuse erythematosus or urticarial-like lesions. Id. Another symptom is arthritis. Id. The number of sites of the body affected by the arthritis vary but affect both the small and large joints in a nearly symmetrical manner. Id. Some patients may also have adenopathy and/or hepatosplenomegaly. Id. Other symptoms such as pericarditis, pleural effusion, or serous peritonitis with abdominal pain may be present. Id. SoJIA patients typically have severe inflammatory disease with a large increase in ferritin levels and a decrease in the percentage of glycosylated ferritin. Id. Physicians often use a “clinical triad” to diagnose this disease: daily fever lasting more than 2 weeks, arthritis, and cutaneous eruptions. See Petty, R et al., J. Rheumatol. 31(2) 390-392 (2004). If the patient does not have cutaneous eruptions, the presence of an adenopathy, hepatosplenomegaly, or serous effusion can also confirm the diagnosis.

IL-18 is a pro-inflammatory cytokine that plays a role in both innate and acquired immune responses. In many patients with AOSD, IL-18 is markedly elevated. Concentrations of IL-18 have been found to be > 100,000 pg/ml in active AOSD and correlate with disease severity. (Kudela, H, et al., BMC Rheum 3:4 (2019)). The elevation appears to be more specific for AOSD than for other systemic rheumatic diseases (Kawashima, M, et al., Arthritis Rheum. 44(3):550 (2001); Kudela, H., et al., BMC Rheum 3:4 (2019)).

A 12-week study of 23 AOSD patients treated with tadekinig alfa (an IL-18 binding protein [IL-18 BP]) showed moderate efficacy in approximately half of the patients treated, suggesting that targeting IL-18 in AOSD may be an effective treatment strategy (Gabay, C, et al. Ann Rheum Dis. Jun;77(6):840-847 (2018)). Limitations of the study design, as well as the dose route (SC) and PK (relatively short t½) of tadekinig alfa may have limited the efficacy seen in this trial.

### BRIEF DESCRIPTION OF THE DRAWINGS

FIG. 1. Key markers of inflammation — C-reactive protein (CRP) measured in mg/L by visit. The Y-axis is CRP concentration measured in mg/L. The X-axis refers to time. Each line refers to data from one patient. Each patient is also assigned a number. Each number next to each data point on the line graph indicates the CRP concentration in mg/L for that patient at that point in time. “V” refers to “visit” and the number behind the “V” refers to the visit number. For example, “V3” refers to visit 3. “Early Term” refers to early termination. “F/U” refers to follow-up visit. The weeks on the X-axis (e.g., “Wk 1”) refer to assessment weeks as shown in Table 1, Schedule of Assessments, herein.

FIGS. 2A and 2B. FIG. 2A shows key markers of inflammation - ferritin measured in ng/L by visit for two patients. The Y-axis is ferritin concentration in ng/mL. The X-axis refers to time. Each line refers to data from one patient. Each patient is also assigned a number. Each number next to each data point on the line graph indicates the ferritin concentration in ng/L for that patient at that point in time. “V” refers to “visit” and the number behind the “V” refers to the visit number. For example, “V3” refers to visit 3. The weeks on the X-axis (e.g., “Wk 1”) refer to assessment weeks as shown in Table 1, Schedule of Assessments, herein. “Early Term” refers to early termination. “F/U” refers to follow-up visit. FIG. 2B shows key markers of inflammation - ferritin measured in ng/L by visit for one patient, patient 012-01. The Y-axis is ferritin concentration in ng/mL. The X-axis refers to time. The line refers to data from one patient. The patient is assigned a number. Each number next to each data point on the line graph indicates the ferritin concentration in ng/L for the patient at that point in time. “V” refers to “visit” and the number behind the “V” refers to the visit number. For example, “V3” refers to visit 3. The weeks on the X-axis (e.g., “Wk 1”) refer to assessment weeks as shown in Table 1, Schedule of Assessments, herein. The data for patient 012-01 is shown separately in this figure because that patient’s ferritin concentrations were higher than that of the other patients whose data is shown in FIG. 2A, and required a different scale on the Y-axis.

FIG. 3. Key markers of inflammation - modified Pouchot score by visit. The Y-axis is modified Pouchot score. The X-axis refers to time. Each line refers to data from one patient. Each patient is also assigned a number. “V” refers to “visit” and the number behind the “V” refers to the visit number. For example, “V3” refers to visit 3. “ET” refers to early termination. The weeks on the X-axis (e.g., “Wk 1”) refer to assessment weeks as shown in Table 1, Schedule of Assessments, herein.

FIG. 4. Key markers of inflammation - physician global assessment (PhGA) measured on a visual analog scale (VAS) of 100 mm by visit. The Y-axis is PhGA score measured on a VAS of 100 mm. The X-axis refers to time. Each line refers to data from one patient. Each patient is also assigned a number. “V” refers to “visit” and the number behind the “V” refers to the visit number. For example, “V3” refers to visit 3. “ET” refers to early termination. The weeks on the X-axis (e.g., “Wk 1”) refer to assessment weeks as shown in Table 1, Schedule of Assessments, herein.

FIG. 5. Key markers of inflammation - patient global assessment (PtGA) measured on a visual analog scale (VAS) of 100 mm by visit. The Y-axis is PtGA score measured on a VAS of 100 mm. The X-axis refers to time. Each line refers to data from one patient. Each patient is also assigned a number. Each number next to each data point on the line graph indicates the PtGA score for that patient at that point in time. “V” refers to “visit” and the number behind the “V” refers to the visit number. For example, “V3” refers to visit 3. “ET” refers to early termination. The weeks on the X-axis (e.g., “Wk 1”) refer to assessment weeks as shown in Table 1, Schedule of Assessments, herein.

### SUMMARY

The present disclosure includes, for example, any one or a combination of the following embodiments:

Embodiment 1. A method of treating Adult-onset Still’s disease (AOSD) or systemic-onset juvenile idiopathic arthritis (SoJIA), comprising administering to a human subject diagnosed with AOSD or SoJIA an effective amount of an anti-IL-18 antibody wherein the anti-IL-18 antibody comprises the following six CDRs:
(a) a HCDR1 having an amino acid sequence of SEQ ID NO: 122;
(b) a HCDR2 having an amino acid sequence of SEQ ID NO: 123;
(c) a HCDR3 having an amino acid sequence of SEQ ID NO: 124;
(d) a LCDR1 having an amino acid sequence of SEQ ID NO: 126;
(e) a LCDR2 having an amino acid sequence of SEQ ID NO: 127; and
(f) a LCDR3 having an amino acid sequence of SEQ ID NO: 128;
and wherein the anti-IL-18 antibody is administered at a dose of 4 mg/kg, 7 mg/kg, or 14 mg/kg.

Embodiment 2. The method of embodiment 1, wherein the subject is diagnosed with AOSD based on having 5 or more of the following criteria, 2 of which are major:
(a) Major Criteria
   i. Fever >39° C., lasting 1 week or longer
   ii. Arthralgia or arthritis, lasting 2 weeks or longer
   iii. Typical rash
   iv. Leukocytes >10,000 mm³ with >80% polymorphonuclear cells
(b) Minor Criteria
   v. Sore throat
   vi. Recent development of significant lymphadenopathy
   vii. Hepatomegaly or splenomegaly
   viii. Abnormal liver function tests
   ix. Negative tests for antinuclear antibody (IF) and rheumatoid factor (IgM).

Embodiment 3. The method of embodiment 1 or embodiment 2, wherein the subject has reported a recurring fever >38° C. within the last 5 days of screening and baseline visits.

Embodiment 4. The method of any one of embodiments 1-3, wherein if the subject is undergoing treatment with NSAIDs, the subject is on a stable dose for at least 48 hours prior to a baseline visit.

Embodiment 5. The method of any one of embodiments 1-4, wherein if the subject is undergoing treatment with glucocorticoids, the subject is on a stable dose for at least 48 hours prior to a baseline visit.

Embodiment 6. The method of any one of embodiments 1-5, wherein if the subject is undergoing treatment with conventional DMARDs, the subject is on a stable dose for at least 12 weeks prior to a baseline visit.

Embodiment 7. The method of any one of embodiments 1-6, wherein if the subject has received treatment with biological DMARDs, the subject has undergone the required washout period prior to a baseline visit, wherein the required washout period is as follows:
a) anakinra - 1 week;
b) etanercept, rilonacept - 4 weeks;
c) adalimumab, certolizumab, infliximab, golimumab, abatacept, tocilizumab and canakinumab - 8 weeks; and
d) rituximab - 36 weeks.

Embodiment 8. The method of any one of embodiments 1-7, wherein the subject does not have a serum creatinine concentration of >1.5 mg/dl.

Embodiment 9. The method of any one of embodiments 1-8, wherein the subject does not have hemoglobin ≤ 10 g/dl, neutrophils ≤1,500/µl and/or thrombocytes ≤75,000/µl.

Embodiment 10. The method of any of embodiments 1 or 3-9, comprising a method of treating SoJIA.

Embodiment 11. The method of any one of embodiments 1-10, wherein the subject has elevated free or total serum IL-18 levels.

Embodiment 12. The method of any one of embodiments 1-11, wherein the level of serum IL-18 is measured prior to administration of the anti-IL-18 antibody.

Embodiment 13. The method of any one of embodiments 1-12, wherein the level of serum IL-18 is measured after administration of the anti-IL-18 antibody, optionally as a marker for effectiveness of treatment.

Embodiment 14. The method of any one of embodiments 1-13, wherein the level of serum IL-18 is elevated as compared to levels in a subject without AOSD or SoJIA or as compared to a negative control.

Embodiment 15. The method of any one of embodiments 1-14, wherein levels of serum IL-18 in the subject are measured after administration of the anti-IL-18 antibody as a marker for effectiveness of treatment.

Embodiment 16. The method of any one of embodiments 1-15, wherein the level of serum IL-18 is free IL-18, optionally wherein the level of free IL-18 is calculated.

Embodiment 17. The method of any one of embodiments 1-15, wherein the level of serum IL-18 is total IL-18.

Embodiment 18. The method of any one of embodiments 1-16, wherein the subject has elevated serum free IL-18.

Embodiment 19. The method of any one of embodiments 1-15 or 17, wherein the subject has elevated serum total IL-18.

Embodiment 20. The method of any one of embodiments 1-19, wherein the anti-IL-18 antibody comprises a VH domain having an amino acid sequence that is at least 90% identical to the full sequence of SEQ ID NO: 121.

Embodiment 21. The method of any one of embodiments 1-20, wherein the anti-IL-18 antibody comprises a VH domain having an amino acid sequence that is identical to the full sequence of SEQ ID NO: 121.

Embodiment 22. The method of any one of embodiments 1-21, wherein the anti-IL-18 antibody comprises a VL domain having an amino acid sequence that is at least 90% identical to the full sequence of SEQ ID NO: 125.

Embodiment 23. The method of any one of embodiments 1-22, wherein the anti-IL-18 antibody comprises a VL domain having an amino acid sequence that is identical to the full sequence of SEQ ID NO: 125.

Embodiment 24. The method of any one of embodiments 1-23, wherein the anti-IL-18 antibody comprises an antibody VH domain and an antibody VL domain, wherein the amino acid sequence of the antibody VH domain is at least 90% identical to the full sequence of SEQ ID NO: 121, and the antibody VL domain is at least 90% identical to the full sequence of SEQ ID NO: 125.

Embodiment 25. The method of any one of embodiments 1-24, wherein the anti-IL-18 antibody is administered in the form of a pharmaceutically acceptable composition.

Embodiment 26. The method of any one of embodiments 1-25, wherein the anti-IL-18 antibody is administered for a period of at least 16 weeks.

Embodiment 27. The method of any one of embodiments 1-26, wherein the anti-IL-18 antibody is administered intravenously.

Embodiment 28. The method of any one of embodiments 1-26, wherein the anti-IL-18 antibody is administered subcutaneously.

Embodiment 29. The method of any one of embodiments 1-28, wherein the anti-IL-18 antibody is administered once per week, once every two weeks, once every three weeks, once every four weeks, once every five weeks, or once every six weeks.

Embodiment 30. The method of any one of embodiments 1-29, wherein the subject achieves resolution of fever.

Embodiment 31. The method of any one of embodiments 1-30, wherein the subject achieves a reduction of CRP.

Embodiment 32. The method of any one of embodiments 1-31, wherein the subject achieves a reduction of CRP of ≥50% from baseline to week 4.

Embodiment 33. The method of any one of embodiments 1-32, wherein the subject achieves a reduction of CRP of ≥50% from baseline to week 12.

Embodiment 34. The method of any one of embodiments 1-33, wherein the subject achieves a reduction of serum IL-18 levels.

Embodiment 35. The method of any one of embodiments 1-34, wherein the subject achieves a reduction of serum total IL-18 levels.

Embodiment 36. The method of any one of embodiments 1-34, wherein the subject achieves a reduction of serum free IL-18 levels.

Embodiment 37. The method of any one of embodiments 1-36, wherein the subject has undetectable levels of serum IL-18 at about 4 weeks after administration of the anti-IL-18 antibody.

Embodiment 38. The method of any one of embodiments 1-37, wherein the subject has undetectable levels of serum IL-18 at about 12 weeks after administration of the anti-IL-18 antibody.

Embodiment 39. The method of any one of embodiments 1-38, wherein the subject has undetectable levels of serum total IL-18 at about 4 weeks after administration of the anti-IL-18 antibody.

Embodiment 40. The method of any one of embodiments 1-39, wherein the subject has undetectable levels of serum total IL-18 at about 12 weeks after administration of the anti-IL-18 antibody.

Embodiment 41. The method of any one of embodiments 1-38, wherein the subject has undetectable levels of serum free IL-18 at about 4 weeks after administration of the anti-IL-18 antibody.

Embodiment 42. The method of any one of embodiments 1-39, wherein the subject has undetectable levels of serum free IL-18 at about 12 weeks after administration of the anti-IL-18 antibody.

Embodiment 43. The method of any one of embodiments 1-42, wherein the subject exhibits a reduction of the signs and symptoms of AOSD or SoJIA as measured by the Physician and/or Patient Global Assessment of disease activity assessed on a Visual Analogue Scale (VAS).

Embodiment 44. The method of any one of embodiments 1-43, wherein the subject exhibits a reduction of the signs and symptoms of AOSD or SoJIA as measured by the Physician and/or Patient Global Assessment of disease activity assessed on a Visual Analogue Scale (VAS) at least at about 4 weeks after administration of the anti-IL-18 antibody.

Embodiment 45. The method of any one of embodiments 1-44, wherein the subject exhibits a reduction of the signs and symptoms of AOSD or SoJIA as measured by the Physician and/or Patient Global Assessment of disease activity assessed on a Visual Analogue Scale (VAS) at least at about 12 weeks after administration of the anti-IL-18 antibody.

Embodiment 46. The method of any one of embodiments 1-45, wherein the subject exhibits a reduction of the signs and symptoms of AOSD or SoJIA assessed using the modified Pouchot score.

Embodiment 47. The method of any one of embodiments 1-46, wherein the subject exhibits a reduction of the signs and symptoms of AOSD or SoJIA assessed using the modified Pouchot score at about 4 weeks after administration of the anti-IL-18 antibody.

Embodiment 48. The method of any one of embodiments 1-47, wherein the subject exhibits a reduction of the signs and symptoms of AOSD or SoJIA assessed using the modified Pouchot score at about 12 weeks after administration of the anti-IL-18 antibody.

Embodiment 49. The method of any one of embodiments 1-48, wherein the subject exhibits a reduction of the signs and symptoms of AOSD or SoJIA assessed using the DAS28-CRP score.

Embodiment 50. The method of any one of embodiments 1-49, wherein the subject exhibits a reduction of the signs and symptoms of AOSD or SoJIA assessed using the DAS28-CRP score at about 4 weeks after administration of the anti-IL-18 antibody.

Embodiment 51. The method of any one of embodiments 1-50, wherein the subject exhibits a reduction of the signs and symptoms of AOSD or SoJIA assessed using the DAS28-CRP score at about 12 weeks after administration of the anti-IL-18 antibody.

Embodiment 52. The method of any one of embodiments 1-51, wherein the subject experiences a reduction in serum ferritin.

Embodiment 53. The method of any one of embodiments 1-52, wherein the subject experiences a reduction in serum ferritin at about 4 weeks after administration of the anti-IL-18 antibody.

Embodiment 54. The method of any one of embodiments 1-53, wherein the subject experiences a reduction in serum ferritin at about 12 weeks after administration of the anti-IL-18 antibody.

Embodiment 55. The method of any one of embodiments 1-54, wherein the subject experiences a reduction in erythrocyte sedimentation rate (ESR).

Embodiment 56. The method of any one of embodiments 1-55, wherein the subject experiences a reduction in erythrocyte sedimentation rate (ESR) at about 4 weeks after administration of the anti-IL-18 antibody.

Embodiment 57. The method of any one of embodiments 1-56, wherein the subject experiences a reduction in erythrocyte sedimentation rate (ESR) at about 12 weeks after administration of the anti-IL-18 antibody.

Embodiment 58. The method of any one of embodiments 1-57, further comprising administering one or more additional therapeutically active agents.

Embodiment 59. The method of embodiment 58, wherein the one or more additional therapeutically active agents comprises at least one of non-steroidal anti-inflammatory drugs (NSAIDs), corticosteroids, systemic glucocorticoids, and conventional synthetic disease-modifying anti-rheumatic drugs (DMARDs).

Embodiment 60. The method of embodiment 59, wherein the conventional synthetic disease-modifying anti-rheumatic drug (DMARD) is methotrexate.

Embodiment 61. The method of embodiment 59, wherein the corticosteroid is prednisone.

Embodiment 62. A kit for use in a method of any one of embodiments 1-61, comprising an anti-IL-18 antibody and reagents for carrying out the method.

### DETAILED DESCRIPTION OF THE INVENTION

The following definitions are provided to facilitate an understanding of the invention. They are not intended to limit the invention in any way

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an “isolated,” or “biologically pure” molecule is a compound that has been removed from its natural milieu. As such, the terms “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“IL-18” or “Interleukin-18” or “interferon-gamma inducing factor” or “IFN-γ-inducing factor” refers to a proinflammatory cytokine encoded by the IL18 gene that belongs to the IL-1 family of cytokines. Similar to IL-1β, it is synthesized as an inactive precursor called pro-IL-18 that is activated by cleavage by caspase-1. Pro-IL-18 is present in healthy cells and constitutively expressed by monocytes and epithelial cells. IL-18 has roles in stimulating both adaptive and innate immune response.

“Elevated IL-18” as used herein refers to a level of total IL-18 detected in a subject that is higher than a normal control. The normal control can be determined by those of skill in the art as applicable to the particular situation. In some instances, the normal control is an industry standard agreed upon by those of skill as being a level or range of levels that is typical of an individual without an IL-18-associated condition. In some instances, the normal control is a reference level of IL-18 from the same individual taken at a time point, and whether the subject has elevated IL-18 is determined based on a sample from that same individual taken at a different, typically later, time point.

“Free IL-18” or “free (active) IL-18” herein refers to non-bound form IL-18, which is the active form of IL-18. In humans, free IL-18 is neutralized (inactivated) by IL-18BP, which binds IL-18 and inhibits its activity by interfering with its interaction with IL-18Rα.

“Bound IL-18,” or the like, refers to IL-18 that is bound to a natural ligand, optionally wherein the natural ligand is IL-18Rα or IL-18BP. Free IL-18 may be calculated according to known methods, e.g., Palladino et al. (2012) J. Neuroinflammation 9(206).

“Total IL-18,” or the like, refers to the total amount of free IL-18 and bound IL-18.

“Serum” or “circulating” IL-18 is IL-18 that is located in the serum.

“Undetectable levels of serum IL-18” refers to levels of IL-18 that are below the level of quantification of an assay for measuring IL-18.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3. Unless otherwise stated, a “set of CDRs” includes HCDRs and LCDRs. The term antibody includes, but is not limited to, fragments that are capable of binding antigen, such as Fv, single-chain Fv (scFv), Fab, Fab′, and (Fab′)2. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” or “VH domain” refers to a region comprising a heavy chain complementarity determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “light chain variable region” or “VL domain” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)2, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on human immunoglobulin sequences.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGNTM (DNASTAR) software. Those skilled in the art can determine appropriate parameters for measuring alignment, including any algorithms needed to achieve maximal alignment over the full length of the sequences being compared.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Sample” or “subject sample” or “biological sample” generally refers to a sample which may be tested for a particular molecule. Samples may include but are not limited to cells, bone marrow, body fluids, including blood, serum, plasma, urine, saliva, stool, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins.

A “subject” can be mammalian. In any of the embodiments involving a subject, the subject can be human. In any of the embodiments involving a subject, the subject can be a cow, pig, monkey, sheep, dog, cat, fish, or poultry.

A “pediatric” subject herein is a human of less than 18 years of age, whereas an “adult” subject is 18 years or older.

The term “pharmaceutically acceptable composition” may refer to a composition comprising the anti-IL-18 antibody in formulations with a wide variety of pharmaceutically acceptable carriers.

The term “pharmaceutically acceptable carrier” refers to refers to an ingredient in a pharmaceutical formulation or composition, other than an active ingredient, which is nontoxic to a subject. A pharmaceutically acceptable carrier includes, but is not limited to, a buffer, excipient, stabilizer, and/or preservative.

“Treatment” or “treat” refers to both therapeutic treatment and prophylactic or preventative measures. Those in need of treatment include those already with the disorder as well as those prone to have the disorder or those in which the disorder is to be prevented. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

“Adult-onset Still’s disease” or “AOSD” can be diagnosed in several different ways. One accepted way to diagnose AOSD is based on classification criteria defined as having at least five or more of fever > 39° C., lasting 1 week or longer; arthralgia or arthritis, lasting 2 weeks or longer; typical rash; leukocytes > 10,000 mm³ with >80% polymorphonuclear cells; sore throat; recent development of significant lymphadenopathy; hepatomegaly or splenomegaly; abnormal liver function tests; negative tests for antinuclear antibody (IF) and rheumatoid factor (IgM); and at least two or more of fever > 39° C., lasting 1 week or longer; arthralgia or arthritis, lasting 2 weeks or longer; typical rash; or leukocytes > 10,000 mm³ with >80% polymorphonuclear cells.

The term “PtGA” or “Patient Global Assessment of Disease Activity” measured on a visual analog scale (VAS) is a measure of the patient’s disease activity as assessed by the patient on a 100 mm scale. For example, some Patient Global assessment of Disease Activity scales ask the patient to evaluate their disease activity based on a question such as: “Considering all the ways your AOSD affects you, rate how well you are doing on the following scale” of 0 to 100. A score of “0 mm” often means the patient is doing very well /has no evidence of disease and a score of “100 mm” indicates the patient is doing very poorly/has very severe disease activity.

The term “PhGA” or Physician Global Assessment of Disease Activity″ measured on a visual analog scale (VAS) is a measure of a patient’s disease activity as assessed by a physician on a 100 mm scale. A score of “0 mm” often means the patient is doing very well/has no evidence of disease and a score of “100 mm” indicates the patient is doing very poorly/has very severe disease activity. The physician performing the assessment must not be aware of the specific PtGA when performing his/her own assessment of the patient.

“CRP” refers to C-reactive protein, a protein in the blood. It is made by the liver and it is sent into the bloodstream in response to inflammation. It is measured in mg/L. CRP levels rise and fall depending on inflammation levels, with greater inflammation corresponding to higher CRP levels. Results are typically compared to the normal value ranges, but the normal range values may vary slightly among different laboratories.

“DAS28-CRP” or “Disease Activity Score-28 for Rheumatoid Arthritis with CRP” refers to a score calculated based on information about the following disease variables: the number of swollen joints and tender joints assessed using 28-joint count (TJC28 and SJC28); PtGA (Patient Global Assessment of Disease Activity) measured on a VAS (visual analog scale) of 100 mm; and CRP measured in mg/L. Using this information, the DAS28-CRP score is calculated using the following formula: 0.56 * sqrt(TJC28) + 0.28 * sqrt(SJC28) + 0.36 * ln(CRP+1) + 0.014 * PtGA + 0.96. The DAS28 score ranges from 0 to 10, with higher scores indicating a more severe current activity of the disease. The DAS28 score may also be categorized into disease activity states according to the cut-off values as follows: high disease activity: >5.1; moderate disease activity: ≥3.2 to ≤5.1; low disease activity: >2.6 to <3.2; remission: ≤2.6.

The term “corticosteroids” refers to a type of anti-inflammatory drug. They are man-made drugs that closely resemble cortisol, a hormone that the adrenal glands naturally produce. A few examples of corticosteroids include cortisone and prednisone. Glucocorticosteroids (also referred to as glucocorticoids) are a type of corticosteroid.

The term “disease-modifying anti-rheumatic drugs or “DMARDs” refers to a class of drugs indicated for the treatment of inflammatory arthritides such as rheumatoid arthritis, psoriatic arthritis, and ankylosing spondylitis, and can also be used in the treatment of other disorders such as the connective tissue diseases systemic sclerosis, systemic lupus erythematosus, and Sjogren syndrome. They can also be used in the treatment of inflammatory myositis, vasculitis, uveitis, inflammatory bowel disease, and in some types of cancer. DMARDS are immunosuppressive or immunomodulatory. Each DMARD has a unique mechanism of action, but they all ultimately interfere with any one of many critical pathways of the inflammatory cascade. DMARDS can be conventional or biologic. Common conventional DMARDs include methotrexate, leflunomide, hydroxychloroquine, and sulfasalazine. Biologic DMARDs include anakinra, infliximab, adalimumab, certolizumab, canakinumab, golimumab, etanercept, rilonacept, abatacept, rituximab, tocilizumab, and others.

The term “erythrocyte sedimentation rate” or “ESR” refers to the result of a blood test that helps detect inflammation. The test measures the rate of fall (sedimentation) of red blood cells (erythrocytes) in a sample of blood placed in a tall vertical tube. Increased ESR indicates inflammation. Results are measured in millimeters of plasma left at the top of the tube after one hour. Results are typically compared to the normal value ranges for persons of the same sex and age, but the normal range values may vary slightly among different laboratories.

The term “ferritin” as used herein refers to serum ferritin, which is ferritin in the blood. Ferritin is a protein that stores iron in cells. Results are typically compared to the normal value ranges for persons of the same sex and age, but the normal range values may vary slightly among different laboratories.

“Resolution of fever” as used herein means no temperature >37.5° C. for 48 hours.

The term or “NSAIDs” refers to “non-steroidal anti-inflammatory drugs,” which are commonly used for pain relief and to reduce fever. Common NSAIDs include aspirin, ibuprofen, and naproxen sodium. Other NSAIDs include celecoxib, diclofenac, fenoprofen, indomethacin, ketorolac tromethamine, meclofenamate sodium, diflunisal, tolmetin, ketoprofen, and flurbiprofen.

“Modified Pouchot score” refers to a score that can indicate whether a patient has active disease. For each of the following 12 clinical conditions, a score count of 1 is assigned for any condition that is present at the time of assessment: fever; evanescent rash; pharyngitis; arthritis; myalgia; pleuritis; pericarditis; pneumonitis; lymphadenopathy; hepatomegaly or elevated liver enzymes; leukocyte count > 15,000/µl; ferritin > 3000 µg/l. A total score of >4 is considered to indicate active disease.

“Systemic-onset juvenile idiopathic arthritis” or “systemic juvenile idiopathic arthritis” or “SoJIA” or “SJIA” or “sJIA” refers to a subset of juvenile idiopathic arthritis marked by more severe extra-articular manifestations (fever, cutaneous eruptions). It represents 10-11% of cases of juvenile idiopathic arthritis. Onset usually occurs between 3 and 5 years of age. The clinical signs include fever with oscillating temperatures over a 24-hour period and peaks of over 39° C. or more, which are associated with transient cutaneous eruptions and diffuse erythematosus or urticarial-like lesions. Another symptom is arthritis. The number of sites of the body affected by the arthritis vary but affect both the small and large joints in a nearly symmetrical manner. Some patients may also have adenopathy and/or hepatosplenomegaly. Other symptoms such as pericarditis, pleural effusion, or serous peritonitis with abdominal pain may be present. SoJIA patients typically have severe inflammatory disease with a large increase in ferritin levels and a decrease in the percentage of glycosylated ferritin. Physicians often use a “clinical triad” to diagnose this disease: daily fever lasting more than 2 weeks, arthritis, and cutaneous eruptions. See Petty, R et al., J. Rheumatol. 31(2) 390-392 (2004). If the patient does not have cutaneous eruptions, the presence of an adenopathy, hepatosplenomegaly, or serous effusion can also confirm the diagnosis.

### Anti-IL-18 Antibodies

In some embodiments, the anti-IL-18 antibody utilized for therapeutic purposes described herein comprises the CDR sequences and in some embodiments the heavy and light chain variable regions of Antibody 12_GL disclosed in WO 2012/085015, which is incorporated herein by reference in its entirety. Antibody 12_GL is referred to herein as Antibody A. Antibody A is a fully human IgG1κ monoclonal antibody (mAb) binding and neutralizing IL-18.

Antibody A inhibits the formation of IL-18/Rα/Rβ active complex in-vitro and in-vivo. Antibody A demonstrates high affinity of 63 pM, which is 6 fold higher compared to IL-18’s native inhibitor (IL-18 binding protein), and reduced Fc binding, to enable efficient anti-inflammatory role. As a fully human antibody, Antibody A is expected to demonstrate reduced anti-drug antibodies (ADA). Antibody A demonstrates IL-18 neutralization and bioactivity by reducing IL-18′s effects in various in vitro models with IC50 of sub nanomolar, and has proven efficient in COPD model (NHBE cells infected with Human Rhinovirus (HRV) by inhibiting IFN-y release from PBMCs exposed to infected NHBE media). Antibody A has undergone 13 weeks IV toxicity studies complete with no toxicity up to 100 mg/kg/week.

In some embodiments, the anti-IL-18 antibody comprises the CDRs of Antibody A: (a) a HCDR1 having an amino acid sequence identical to or comprising the amino acids of SEQ ID NO: 122; (b) a HCDR2 having an amino acid sequence identical to or comprising the amino acids of SEQ ID NO: 123; (c) a HCDR3 having an amino acid sequence identical to or comprising the amino acids of SEQ ID NO: 124; (d) a LCDR1 having an amino acid sequence identical to or comprising the amino acids of SEQ ID NO: 126; (e) a LCDR2 having an amino acid sequence identical to or comprising the amino acids of SEQ ID NO: 127; and (f) a LCDR3 having an amino acid sequence identical to or comprising the amino acids of SEQ ID NO: 128.

In some embodiments, the anti-IL-18 antibody comprises the Antibody A VH domain (SEQ ID NO: 121) which may be paired with the Antibody A VL domain (SEQ ID NO: 125), so that an antibody antigen-binding site is formed comprising both the Antibody A VH and VL domains.

In some embodiments, an anti-IL-18 antibody is utilized for both the detection/diagnostic and therapeutic purposes described herein. In one embodiment, the anti-IL-18 antibody used for detection or diagnostic purposes is different from the antibody used for therapeutic purposes (even in the same subject).

In some embodiments, the anti-IL-18 antibody useful for therapeutic purposes may comprise the CDR sequences or heavy and light chain variable regions of the antibodies disclosed in WO 2012/085015, which is incorporated herein by reference in its entirety. The antibodies of WO 2012/085015 referred to herein include Antibody 1, Antibody 1_GL, Antibody 2, Antibody 3, Antibody 4, Antibody 5, Antibody 6, Antibody 6_GL, Antibody 7, Antibody 7_GL, Antibody 8_GL, Antibody 9, Antibody 10, Antibody 11, Antibody 11_GL, and Antibody A.

The anti-IL-18 antibody useful for therapeutic purposes may alternatively comprise the CDR sequences of other anti-IL-18 antibodies known in the art (see for example US6706487, WO 2001/058956, EP 1621616, US 2005/0147610; EP 0 974 600; and WO 0158956).

The anti-IL-18 antibody may alternatively comprise the CDR sequences or heavy and light chain variable regions of any of the antibodies disclosed in US 8,133,978 B2, which is incorporated herein by reference in its entirety. The antibodies of US 8,133,978 B2 referred to herein include the humanized anti-IL-18 antibodies referred to in claims 1 through 7 of US 8,133,978 B2. In some embodiments, the anti-IL-18 antibody is GSK1070806, which is a humanized IgG1/kappa antibody that binds to human IL-18 with a high affinity (Kd = 30.3 pM) and neutralizes its function. See, e.g., Reid, P. et al, Int J Clin Pharmacol Ther. 2014 Oct;52(10):867-79. doi: 10.5414/CP202087.

In some embodiments, the anti-IL-18 antibody of the invention inhibits the binding of IL-18 to one or both of the IL-18 receptor (IL-18R, which comprises IL-18Rα/IL-18Rβ) and IL-18BP and thereby reduces IL-18 activity. In some embodiments, the anti-IL-18 antibody may bind to an epitope on the IL-18 molecule which wholly or partially overlaps the IL-18BP binding site.

For example, the anti-IL-18 antibody may specifically bind to an epitope of IL-18 which comprises one or more of residues Tyr1, Gly3, Leu5, Glu6, Lys8, Met51, Lys53, Asp54, Ser55, Gln56, Pro57, Arg58, Gly59, Met60, Arg104, Ser105 and Pro107 of human IL-18 or the corresponding residues from IL-18 of other species, for example a primate such as Rhesus macaque. The anti-IL-18 antibody may bind to an IL-18 epitope which comprises 1, 2, 3, 4, 5, 6, 7, 8, 9, 10, 11, 12, 13, 14, 15, 16 or all 17 residues selected from the group consisting of Tyr1, Gly3, Leu5, Glu6, Lys8, Met51, Lys53, Asp54, Ser55, Gln56, Pro57, Arg58, Gly59, Met60, Arg104, Ser105, and Pro107 of human IL-18.

In some embodiments, an anti-IL-18 antibody useful in the methods described herein may comprise one or more CDRs as described herein, e.g. a CDR3, and optionally also a CDR1 and CDR2 to form a set of CDRs. In some embodiments, the CDR or set of CDRs is a CDR or set of CDRs of any of Antibody 1, Antibody 1_GL, Antibody 2, Antibody 3, Antibody 4, Antibody 5, Antibody 6, Antibody 6_GL, Antibody 7, Antibody 7_GL, Antibody 8_GL, Antibody 9, Antibody 10, Antibody 11, Antibody 11_GL, and Antibody A, or may be a variant thereof as described herein.

In some embodiments: HCDR1 may be about 7 amino acids long, comprising or consisting of Kabat residues 31-35b; HCDR2 may be about 16 amino acids long, comprising or consisting of Kabat residues 50-65; HCDR3 may be about 15 amino acids long, comprising or consisting of Kabat residues 95-102; LCDR1 may be about 11 amino acids long, comprising or consisting of Kabat residues 24-34; LCDR2 may be about 7 amino acids long, comprising or consisting of Kabat residues 50-56; and/or LCDR3 may be about 9 amino acids long, comprising or consisting of Kabat residues 89-97. As is known in the art, in the variable region of the heavy chain, the CDR may include residues 31 to 35 plus an insertion of 2 residues, 35a and 35b.

In some embodiments, the anti-IL-18 antibody comprises a HCDR1, HCDR2 and/or HCDR3 and/or an LCDR1, LCDR2 and/or LCDR3 as provided in Table 6 (the CDRs belonging to an individual antibody). The anti-IL-18 antibody may comprise a VH as described in any one of the antibodies in the Table 6. Optionally, it may also comprise a VL of any one of these antibodies. The VL may be from the same or a different antibody as the VH. A VH domain comprising a set of HCDRs of any of the antibodies listed in the Table 6, and/or a VL domain comprising a set of LCDRs of any of the antibodies listed in the Table 6, are also provided herein.

In some embodiments, the anti-IL-18 antibody comprises Antibody A CDRs with amino acid residue substitutions: (a) a HCDR1 having an amino acid sequence identical to or comprising 1, 2, or 3 amino acid residue substitutions relative to SEQ ID NO: 122; (b) a HCDR2 having an amino acid sequence identical to or comprising 1, 2, 3 or 4 amino acid residue substitutions relative to SEQ ID NO: 123; (c) a HCDR3 having an amino acid sequence identical to or comprising 1, 2, 3, 4 or 5 amino acid residue substitutions relative to SEQ ID NO: 124;(d) a LCDR1 having an amino acid sequence identical to or comprising 1, 2, 3 or 4 amino acid residue substitutions relative to SEQ ID NO: 126; (e) a LCDR2 having an amino acid sequence identical to or comprising 1, 2, 3 or 4 amino acid residue substitutions relative to SEQ ID NO: 127; and (f) a LCDR3 having an amino acid sequence identical to or comprising 1, 2, 3, 4, 5, 6, 7, 8, or 9 amino acid residue substitutions relative to SEQ ID NO: 128.

In some embodiments, an anti-IL-18 antibody comprises a heavy chain and/or light chain of a parent antibody. In some embodiments, the anti-IL-18 antibody comprises any of the antibodies listed in the Table 6 with one or more substitutions within the CDRs. In some embodiments, the anti-IL-18 antibody comprises any of the antibodies listed in the Table 6 with one or more substitutions within the VH and/or VL. For example, an antibody molecule of the invention may comprise any one of Antibody 1, Antibody 1_GL, Antibody 2, Antibody 3, Antibody 4, Antibody 5, Antibody 6, Antibody 6_GL, Antibody 7, Antibody 7_GL, Antibody 8_GL, Antibody 9, Antibody 10, Antibody 11, Antibody 11_GL, and Antibody A, with 17, 16 or 15 or fewer substitutions, e.g. 14, 13, 12, 11, 10, 9, 8, 7, 6, 5, 4, 3, 2 or 1 substitutions within the VH and/or VL. Substitutions may potentially be made at any residue, including within the set of CDRs.

Typically, a VH domain is paired with a VL domain to provide an antibody antigen-binding site, although as discussed above a VH or VL domain alone may be used to bind antigen. For example, the Antibody A VH domain (SEQ ID NO: 121) may be paired with the Antibody A VL domain (SEQ ID NO: 125), so that an antibody antigen-binding site is formed comprising both the Antibody A VH and VL domains. Analogous embodiments are provided for the VH and VL domains of the other antibodies disclosed herein.

In other embodiments, the Antibody A VH is paired with a VL domain other than that of Antibody A VL. Light-chain promiscuity is well established in the art. Again, analogous embodiments are provided by the invention for the other VH and VL domains disclosed herein. Thus, the VH of the parent Antibody 1 or of any of the optimised clones Antibody 1_GL, Antibody 2, Antibody 3, Antibody 4, Antibody 5, Antibody 6, Antibody 6_GL, Antibody 7, Antibody 7_GL, Antibody 8_GL, Antibody 9, Antibody 10, Antibody 11, Antibody 11_GL, and Antibody A may be paired with a VL domain from a different antibody e.g. the VH and VL domains may be from different antibodies selected from Antibody 1, Antibody 1_GL, Antibody 2, Antibody 3, Antibody 4, Antibody 5, Antibody 6, Antibody 6_GL, Antibody 7, Antibody 7_GL, Antibody 8_GL, Antibody 9, Antibody 10, Antibody 11, Antibody 11_GL, and Antibody A.

In some embodiments, an anti-IL-18 antibody comprise a VH domain and a VL domain wherein; (i) the VH domain amino acid sequence is shown in SEQ ID NO: 121 and the VL domain amino acid sequence is shown in SEQ ID NO: 125, (ii) the VH domain amino acid sequence has 1, 2, 3, 4, 5, 6, 7, 8, 9, 10, 11, 12, 13, 14 or 15 amino acid substitutions as compared to SEQ ID NO: 121 and the VL domain amino acid sequence has 1, 2, 3, 4, 5, 6, 7, 8, 9, 10, 11, 12 or 13 amino acid substitutions as compared to SEQ ID NO: 125; or (iii) the VH domain amino acid sequence has at least 80%, at least 85%, at least 90% or at least 95% sequence identity with SEQ ID NO: 121 and the VL domain amino acid sequence has at least 80%, at least 85%, at least 90% or at least 95% sequence identity with SEQ ID NO: 125.

In some embodiments, the anti-IL-18 antibody comprises a VH domain having an amino acid sequence which is at least 90% identical to the full sequence of SEQ ID NO: 121. In some embodiments, the anti-IL-18 antibody comprises a VH domain having an amino acid sequence which is identical to the full sequence of SEQ ID NO: 121. In some embodiments, the anti-IL-18 antibody comprises a VL domain having an amino acid sequence which is at least 90% identical to the full sequence of SEQ ID NO: 125. In some embodiments, the anti-IL-18 antibody comprises a VL domain having an amino acid sequence which is identical to the full sequence of SEQ ID NO: 125. In some embodiments, the anti-IL-18 antibody comprises an antibody VH domain and an antibody VL domain, wherein the amino acid sequence of the antibody VH domain and the antibody VL domain are at least 90% identical to the full sequence of SEQ ID NOS: 121 and 125.

In some embodiments, the anti-IL-18 antibody comprises a heavy chain and light chain comprising the following complementarity determining regions (CDRs): (a) a HCDR1 comprising the amino acids of SEQ ID NO: 129; (b) a HCDR2 comprising the amino acids of SEQ ID NO: 130; (c) a HCDR3 comprising the amino acids of SEQ ID NO: 131; (d) a LCDR1 comprising the amino acids of SEQ ID NO: 132; (e) a LCDR2 comprising the amino acids of SEQ ID NO: 133; and (f) a LCDR3 comprising the amino acids of SEQ ID NO: 134.

In some embodiments, the anti-IL-18 antibody of the invention comprises: a heavy chain and light chain comprising the following complementarity determining regions (CDRs): (a) a HCDR1 having an amino acid sequence identical to or comprising 1, 2, or 3 amino acid residue substitutions relative to SEQ ID NO: 129; (b) a HCDR2 having an amino acid sequence identical to or comprising 1, 2, 3 or 4 amino acid residue substitutions relative to SEQ ID NO: 130; (c) a HCDR3 having an amino acid sequence identical to or comprising 1, 2, 3, 4 or 5 amino acid residue substitutions relative to SEQ ID NO: 131; (d) a LCDR1 having an amino acid sequence identical to or comprising 1, 2, 3 or 4 amino acid residue substitutions relative to SEQ ID NO: 132; (e) a LCDR2 having an amino acid sequence identical to or comprising 1, 2, 3 or 4 amino acid residue substitutions relative to SEQ ID NO: 133; and (f) a LCDR3 having an amino acid sequence identical to or comprising 1, 2, 3, or 4 amino acid residue substitutions relative to SEQ ID NO: 134.

In some embodiments, the IL-18 antibody comprises a heavy chain having an amino acid sequence identical to or comprising 1 to 12 amino acid residue substitutions relative to a heavy chain selected from the group consisting of: SEQ ID NO: 137, SEQ ID NO: 145, and SEQ ID NO: 149; and a light chain having an amino acid sequence identical to or comprising 1 to 12 amino acid residue substitutions relative to a light chain selected from the group consisting of: SEQ ID NO: 141 and SEQ ID NO: 157. In some embodiments, the anti-IL-18 antibody further comprises substituting the residue at position 71 of the light chain with the corresponding residue found in a donor antibody from which the CDRs are derived. In some embodiments, the anti-IL-18 antibody comprises a tyrosine at position 71 of the light chain. In some embodiments, the anti-IL-18 antibody comprises a phenylalanine at position 71 of the light chain.

In one embodiment, the anti-IL-18 antibody comprises a heavy chain of SEQ ID NO: 137 and a light chain of SEQ ID NO: 141. In one embodiment, the anti-IL-18 antibody comprises a heavy chain of SEQ ID NO: 145 and a light chain of SEQ ID NO: 141. In one embodiment, the anti-IL-18 antibody comprises a heavy chain of SEQ ID NO: 149 and a light chain of SEQ ID NO: 141. In one embodiment, the anti-IL-18 antibody comprises a heavy chain of SEQ ID NO: 137 and a light chain of SEQ ID NO: 157. In one embodiment, the anti-IL-18 antibody comprises a heavy chain of SEQ ID NO: 145 and a light chain of SEQ ID NO: 157. In one embodiment, the anti-IL-18 antibody comprises a heavy chain of SEQ ID NO: 149 and a light chain of SEQ ID NO: 157. In one embodiment, the anti-IL-18 antibody comprises a heavy chain of SEQ ID NO: 137 and a light chain of SEQ ID NO: 153. In one embodiment, the anti-IL-18 antibody the anti-IL-18 antibody comprises a heavy chain of SEQ ID NO: 145 and a light chain of SEQ ID NO: 153. In one embodiment, the anti-IL-18 antibody the anti-IL-18 antibody comprises a heavy chain of SEQ ID NO: 149 and a light chain of SEQ ID NO: 153.

In some embodiments, the anti-IL-18 antibody may lack antibody constant regions, for example a scFv.

In other embodiments, anti-IL-18 antibody may comprise an antibody constant region. The anti-IL-18 antibody may be a whole antibody such as an IgG, i.e. an IgG1, IgG2, or IgG4, or may be an antibody fragment or derivative as described below. Antibody molecules can also have other formats, e.g. IgG1 with YTE (Dall’ Acqua et al. (2002) J. Immunology, 169: 5171-5180; Dall’ Acqua et al. (2006) J Biol. Chem. 281(33):23514-24) and/or TM mutations (Oganesyan et al. (2008) Acta Cryst D64:700-4) in Fc region.

Another aspect of the invention provides an anti-IL-18 antibody comprising an antibody antigen binding site or antibody molecule as described herein which competes for binding to IL-18 with any antibody molecule which: (i) binds IL-18 and (ii) comprises an antibody molecule, VH and/or VL domain, CDR e.g. HCDR3, and/or set of CDRs listed in the Table 6.

For example, in some embodiments, the anti-IL-18 antibody may compete with an antibody molecule comprising: (i) a VH domain having the sequence of SEQ ID NO: 152 and a VL domain having the sequence of SEQ ID NO: 157; (ii) a VH domain having a sequence with 15 or fewer amino acid substitutions such as 14, 13, 12, 11, 10, 9, 8, 7, 6, 5, 4, 3, 2, or 1 as compared to SEQ ID NO: 152; and a VL domain having a sequence with 13 or fewer amino acid substitutions such as 12, 11, 10, 9, 8, 7, 6, 5, 4, 3, 2, or 1 as compared to or SEQ ID NO: 157, or; (iii) a VH domain and a VL domain having sequences with at least 90% sequence identity to SEQ ID NO: 152 and SEQ ID NO: 157, respectively.

Competition between anti-IL-18 antibodies may be assayed easily in vitro, for example using ELISA and/or by a biochemical competition assay such as one tagging a specific reporter molecule to one anti-IL-18 antibody which can be detected in the presence of one or more other untagged anti-IL-18 antibodies, to enable identification of anti-IL-18 antibodies which bind the same epitope or an overlapping epitope. Such methods are readily known to one of ordinary skill in the art and are described in more detail herein.

Variable domain amino acid sequence variants of any of the VH and VL domains whose sequences are specifically disclosed herein may be employed in accordance with the present invention.

As described above, aspects of the invention provide an anti-IL-18 antibody, comprising a VH domain that has at least 75%, at least 80%, at least 85%, at least 90%, at least 93%, at least 95%, at least 96%, at least 95%, at least 97%, at least 98% or at least 99% amino acid sequence identity with a VH domain of any of the antibodies listed herein, for which VH domain sequences are shown in the appended Table 6 below; and/or comprising a VL domain that has at least 75%, at least 80%, at least 85%, at least 90%, at least 93%, at least 95%, at least 96%, at least 95%, at least 97%, at least 98% or at least 99% amino acid sequence identity with a VL domain of any of the antibodies listed herein, for which VL domain sequences are shown in the appended Table 6 below.

Aspects of the invention provide an anti-IL-18 antibody comprising a VH domain having a set of VH CDRs that have at least 75%, at least 80%, at least 85%, at least 90%, at least 93%, at least 95%, at least 96%, at least 95%, at least 97%, at least 98% or at least 99% amino acid sequence identity with the set of VH CDRs of any of the antibodies listed herein, for which VH CDR sequences are shown in the appended Table 6; and/or comprising a VL domain having a set of VL CDRs that have at that has at least 75%, at least 80%, at least 85%, at least 90%, at least 93%, at least 95%, at least 96%, at least 95%, at least 97%, at least 98% or at least 99% amino acid sequence identity with the set of VL CDRs of any of the antibodies listed herein, for which the VL CDR sequences are shown in the appended Table 6.

Algorithms that can be used to calculate % identity of two amino acid sequences are known in the art and include e.g. BLAST [Altschul et al. (1990) J. Mol. Biol. 215: 405-410], FASTA [Pearson and Lipman (1988) PNAS USA 85: 2444-2448], or the Smith-Waterman algorithm [Smith and Waterman (1981) J. Mol Biol. 147: 195-197] e.g. employing default parameters.

### Pharmaceutical Formulations

The anti-IL-18 antibody may be administered in the form of a pharmaceutically acceptable composition. In various embodiments, compositions comprising anti-IL-18 antibodies are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising anti-IL-18 antibodies may be formulated for injection or infusion, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical packs and kits comprising one or more containers, each containing one or more doses of an anti-IL-18 antibody are also provided. In some embodiments, a unit dosage is provided wherein the unit dosage contains a predetermined amount of a composition comprising an anti-IL-18 antibody, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan. Treatment with Anti-IL-18 Antibodies

In some embodiments, a method of treating subjects having adult-onset Still’s disease (AOSD) or systemic-onset juvenile idiopathic arthritis (SoJIA) is provided comprising administering an effective amount of an anti-IL-18 antibody to a human subject diagnosed with AOSD or SoJIA.

In some embodiments, the subject is diagnosed with AOSD based on having 5 or more of the following criteria, 2 of which are major:
(a) Major Criteria
   Fever >39° C., lasting 1 week or longer
   Arthralgia or arthritis, lasting 2 weeks or longer
   Typical rash
   Leukocytes >10,000 mm³ with >80% polymorphonuclear cells
   (b) Minor Criteria
   Sore throat
   Recent development of significant lymphadenopathy
   Hepatomegaly or splenomegaly
   Abnormal liver function tests
   Negative tests for antinuclear antibody (IF) and rheumatoid factor (IgM).

In some embodiments, the subject has reported a recurring fever >38° C. within the last 5 days of screening and baseline visits.

In some embodiments, if the subject is undergoing treatment with NSAIDs, the subject is on a stable dose for at least 48 hours prior to a baseline visit. In some embodiments, if the subject is undergoing treatment with glucocorticoids, the subject is on a stable dose for at least 48 hours prior to a baseline visit. In some embodiments, if the subject is undergoing treatment with conventional DMARDs, the subject is on a stable dose for at least 12 weeks prior to a baseline visit. In some embodiments, if the subject has received treatment with biological DMARDs, the subject has undergone the required washout period prior to a baseline visit, and the required washout period is as follows: anakinra - 1 week; etanercept, rilonacept - 4 weeks; adalimumab, certolizumab, infliximab, golimumab, abatacept, tocilizumab and canakinumab - 8 weeks; and rituximab - 36 weeks.

In some embodiments, the subject does not have a serum creatinine concentration of > 1.5 mg/dl. In some embodiments, the subject does not have hemoglobin ≤ 10 g/dl, neutrophils ≤1,500/µl and/or thrombocytes ≤75,000/µl.

In some embodiments, the method comprises a method of treating SoJIA.

In some embodiments, the subject has elevated free or total serum IL-18 levels. In some embodiments, the level of serum IL-18 is measured prior to administration of the anti-IL-18 antibody. In some embodiments, the level of serum IL-18 is measured after administration of the anti-IL-18 antibody. In some embodiments, the level of serum IL-18 is measured as a marker for effectiveness of treatment. In some embodiments, the level of serum IL-18 is elevated as compared to levels in a subject without AOSD or SoJIA or as compared to a negative control. In some embodiments, the levels of serum IL-18 in the subject are measured after administration of the anti-IL-18 antibody as a marker for effectiveness of treatment. In some embodiments, the level of serum IL-18 is free IL-18. In some embodiments, the level of free IL-18 is calculated. In some embodiments, the level of serum IL-18 is total IL-18. In some embodiments, the subject has elevated serum free IL-18. In some embodiments, the subject has elevated serum total IL-18.

In some embodiments, the anti-IL-18 antibody is administered in the form of a pharmaceutically acceptable composition.

In some embodiments, the anti-IL-18 antibody is administered for a period of at least 16 weeks. In some embodiments, the anti-IL-18 antibody is administered intravenously. In some embodiments, the anti-IL-18 antibody is administered subcutaneously.

In some embodiments, the anti-IL-18 antibody is administered once per week, once every two weeks, once every three weeks, once every four weeks, once every five weeks, or once every six weeks.

In some embodiments, the subject achieves resolution of fever.

In some embodiments, the subject achieves a reduction of CRP. In some embodiments, the subject achieves a reduction of CRP of ≥50% from baseline to week 4. In some embodiments, the subject achieves a reduction of CRP of ≥50% from baseline to week 12.

In some embodiments, the subject achieves a reduction of serum IL-18 levels. In some embodiments, the subject achieves a reduction of serum total IL-18 levels. In some embodiments, the subject achieves a reduction of serum free IL-18 levels. In some embodiments, the subject has undetectable levels of serum IL-18 at about 4 weeks after administration of the anti-IL-18 antibody. In some embodiments, the subject has undetectable levels of serum IL-18 at about 12 weeks after administration of the anti-IL-18 antibody. In some embodiments, the subject has undetectable levels of serum total IL-18 at about 4 weeks after administration of the anti-IL-18 antibody. In some embodiments, the subject has undetectable levels of serum total IL-18 at about 12 weeks after administration of the anti-IL-18 antibody. In some embodiments, the subject has undetectable levels of serum free IL-18 at about 4 weeks after administration of the anti-IL-18 antibody. In some embodiments, the subject has undetectable levels of serum free IL-18 at about 12 weeks after administration of the anti-IL-18 antibody.

In some embodiments, the subject exhibits a reduction of the signs and symptoms of AOSD or SoJIA as measured by the Physician and/or Patient Global Assessment of disease activity assessed on a Visual Analogue Scale (VAS). In some embodiments, the subject exhibits a reduction of the signs and symptoms of AOSD or SoJIA as measured by the Physician and/or Patient Global Assessment of disease activity assessed on a Visual Analogue Scale (VAS) at least at about 4 weeks after administration of the anti-IL-18 antibody. In some embodiments, the subject exhibits a reduction of the signs and symptoms of AOSD or SoJIA as measured by the Physician and/or Patient Global Assessment of disease activity assessed on a Visual Analogue Scale (VAS) at least at about 12 weeks after administration of the anti-IL-18 antibody.

In some embodiments, the subject exhibits a reduction of the signs and symptoms of AOSD or SoJIA assessed using the modified Pouchot score. In some embodiments, the subject exhibits a reduction of the signs and symptoms of AOSD or SoJIA assessed using the modified Pouchot score at about 4 weeks after administration of the anti-IL-18 antibody. In some embodiments, the subject exhibits a reduction of the signs and symptoms of AOSD or SoJIA assessed using the modified Pouchot score at about 12 weeks after administration of the anti-IL-18 antibody.

In some embodiments, the subject exhibits a reduction of the signs and symptoms of AOSD or SoJIA assessed using the DAS28-CRP score. In some embodiments, the subject exhibits a reduction of the signs and symptoms of AOSD or SoJIA assessed using the DAS28-CRP score at about 4 weeks after administration of the anti-IL-18 antibody. In some embodiments, the subject exhibits a reduction of the signs and symptoms of AOSD or SoJIA assessed using the DAS28-CRP score at about 12 weeks after administration of the anti-IL-18 antibody.

In some embodiments, the subject experiences a reduction in serum ferritin. In some embodiments, the subject experiences a reduction in serum ferritin at about 4 weeks after administration of the anti-IL-18 antibody. In some embodiments, the subject experiences a reduction in serum ferritin at about 12 weeks after administration of the anti-IL-18 antibody.

In some embodiments, the subject experiences a reduction in erythrocyte sedimentation rate (ESR). In some embodiments, the subject experiences a reduction in erythrocyte sedimentation rate (ESR) at about 4 weeks after administration of the anti-IL-18 antibody. In some embodiments, the subject experiences a reduction in erythrocyte sedimentation rate (ESR) at about 12 weeks after administration of the anti-IL-18 antibody.

In some embodiments, the subject is administered one or more additional therapeutically active agents. In some embodiments, the one or more additional therapeutically active agents comprise at least one of non-steroidal anti-inflammatory drugs (NSAIDs), corticosteroids, systemic glucocorticoids, and conventional synthetic disease-modifying anti-rheumatic drugs (DMARDs). In some embodiments, the conventional synthetic disease-modifying anti-rheumatic drug (DMARD) is methotrexate. In some embodiments, the corticosteroid is prednisone.

### Free IL-18 Detection Assays

Most currently available assays only measure total IL-18, which includes IL-18 bound to its receptors, including IL-18bp. Total IL-18 may not provide as accurate of a picture of the levels of IL-18 causing disease, which may be free, unbound IL-18. Thus, it may be desirable to use an assay that measures free IL-18 alone when the methods include detection of free IL-18. Alternatively, free IL-18 can be calculated based on total IL-18 and total IL-18bp concentration data using formulas known in the art.

### Kits and Articles of Manufacture

Any of the aforementioned methods can be implemented via kits for the detection and/or treatment of a condition associated with elevated IL-18, including AOSD or SoJIA. The kit may contain an antibody, one or more non-naturally occurring detectable labels, marker, or reporter, a pharmaceutically acceptable carrier, a physiologically acceptable carrier, instructions for use, a container, a vessel for administration, an assay substrate, or any combination thereof.

In some embodiments, the kit is for use in a method of detecting IL-18 in a biological sample. It may contain an anti-IL-18 antibody and reagents for carrying out the method.

In some embodiments, the kit is for use in a method of detecting IL-18 in a biological sample from a subject having or suspected of having AOSD or SoJIA. It may contain an anti-IL-18 antibody and reagents for carrying out the method.

In some embodiments, the kit is for use in method of detecting elevated IL-18 in a biological sample from a subject, optionally wherein the subject is suspected of AOSD or SoJIA and also for treating the subject after diagnosis by administering an anti-IL-18 antibody in an effective amount. It may contain an anti-IL-18 antibody and reagents for carrying out the method.

In some embodiments, the kit for use in a method of detecting and/or treating comprises a solid phase to which the anti-IL-18 antibody reagent is attached. In some embodiments, the kit for use in a method of detecting and/or treating comprises a solid phase to which IL-18 derived from the biological sample will be attached.

The solid phase to be used in the kits of the present invention includes, but is not limited, to microplates, magnetic particles, filter papers for immunochromatography, polymers such as polystyrene, glass beads, glass filters and other insoluble carriers. In one embodiment, a solid substrate containing many compartments or regions has at least one compartment coated with antibodies of the invention.

The kits of the invention may also include a further component to the diagnostic agent, the anti-IL-18 antibody. The further component may include, but is not limited to, reagents, enzymes for labeling, substrates therefor, radioisotopes, light-reflecting substances, fluorescent substances, colored substances, buffer solutions, and plates, and those mentioned herein above.

### EXAMPLES

The following examples are provided to illustrate certain disclosed embodiments and are not to be construed as limiting the scope of this disclosure in any way. In the Examples discussed below, “Antibody A” refers to an anti-IL-18 antibody, wherein the anti-IL-18 antibody comprises the following six CDRs: a heavy chain CDR having an amino acid sequence of SEQ ID NO: 122; a heavy chain CDR having an amino acid sequence of SEQ ID NO: 123; a heavy chain CDR having an amino acid sequence of SEQ ID NO: 124; a light chain CDR having an amino acid sequence of SEQ ID NO: 126; a light chain CDR having an amino acid sequence of SEQ ID NO: 127; and a light chain CDR having an amino acid sequence of SEQ ID NO: 128. In some embodiments Antibody A has a variable heavy chain (VH) having an amino acid sequence of SEQ ID NO: 121 and a variable light chain (VL) having an amino acid sequence of SEQ ID NO: 125.

### Example 1 - A Phase 1b, Multicenter, Open-Label Study to Evaluate the Safety and Tolerability, Efficacy, Pharmacokinetics, and Pharmacodynamics of Antibody A in Subjects with Adult-Onset Still’s Disease

### Example 1.1 - Study Objectives and Endpoints

The primary objective of the study is to evaluate the safety and tolerability of Antibody A at a dose of 7 mg/kg (maximum 500 mg) in subjects with AOSD. It is the first time Antibody A will be studied in subjects with AOSD. Initially, 6 subjects were enrolled at the 7 mg/kg dose. If this dose was found to be tolerable in the first 6 subjects by the Safety Review Committee, the dose would be increased to 14 mg/kg (maximum 1000 mg) in the next set of 6 subjects. Per the predefined algorithm, the Safety Review Committee was given the option to decide to expand the number of subjects in the first cohort before proceeding with the dose escalation in the second cohort of subjects. If the safety of Antibody A at 7 mg/kg dose was not acceptable, dosing would be decreased to a dose of 4 mg/kg (maximum 300 mg) per the predefined algorithm.

The secondary objectives of the study are: to evaluate the effect of Antibody A on the systemic clinical manifestations of AOSD; to measure the effect of Antibody A on the systemic markers of inflammation in subjects with AOSD; and to assess the PK of Antibody A in subjects with AOSD.

The exploratory objectives are to assess the PD of Antibody A in subjects with AOSD and to assess the immunogenicity of Antibody A in subjects with AOSD.

The primary endpoint of the study is incidences of AEs, and changes in vital signs and clinical laboratory results.

The secondary endpoints of the study are: proportion of subjects who achieved resolution of fever, defined as no temperature > 37.5° C. for 48 hours; proportion of subjects whose CRP reduced >50% from Baseline to Week 4 and Week 12; change from baseline to Week 4 and Week 12 in the Physician and Patient Global Assessment of disease activity assessed on a VAS from no evidence of disease (0 mm) to very severe disease activity (100 mm); change from baseline to Week 4 and Week 12 in the modified Pouchot score; change from baseline to Week 4 and Week 12 in the DAS28-CRP; change from baseline to Week 4 and Week 12 in CRP, ferritin, and ESR; and serum concentrations of Antibody A over time.

The exploratory endpoints of the study are change from baseline in IL-18 and IL-18BP levels and incidence of ADAs to Antibody A. IL-18 is considered as an efficient marker for diagnosis and follow-up of AOSD (Jung et al. (2014) Scand J Rheumatol. 43(2):162-169).

### Example 1.2 - Study Design

The study will take place at approximately 15 study sites in the US and Europe.

Approximately 12 subjects aged 18 to 75 years with AOSD are planned to be enrolled. This age range has been chosen considering that AOSD is prevalent in adults as well as subjects aged more than 65 years. The diagnosis of AOSD was based on the classification criteria by Yamaguchi et al. (1992) J Rheumatol. 19(3):424-430, which is the most sensitive classification criteria (Fong & Lui (2013) Proceedings of Singapore Healthcare. 22(1):40-47).

### Inclusion Criteria

Subjects must fulfill the following requirements to be eligible for the study:
2. Subject is 18 to 75 years of age (inclusive) at the time of consent. The date of signature of the informed consent is defined as the beginning of the Screening Period. This inclusion criterion will only be assessed at the Screening Visit (Visit 1).
3. Subject has been diagnosed with AOSD based on classification criteria (according to Yamaguchi et al. (1992) J Rheumatol. 19(3):424-430) defined as having 5 or more of the following criteria, 2 of which are major:
   a. Major Criteria
      i. Fever >39° C., lasting 1 week or longer
      ii. Arthralgia or arthritis, lasting 2 weeks or longer
      iii. Typical rash
      iv. Leukocytes >10,000 mm³ with >80% polymorphonuclear cells
   b. Minor Criteria
      i. Sore throat
      ii. Recent development of significant lymphadenopathy
      iii. Hepatomegaly or splenomegaly
      iv. Abnormal liver function tests
      v. Negative tests for antinuclear antibody (IF) and rheumatoid factor (IgM)
4. Subject has reported a recurring fever >38° C., consistent with active disease, within the last 5 days of the Screening and Baseline visits.
5. If undergoing treatment with NSAIDs, subject is on a stable dose for at least 48 hours prior to the Baseline Visit (Visit 2).
6. If undergoing treatment with glucocorticoids, subject is on a stable dose for at least 48 hours prior to the Baseline Visit (Visit 2).
7. If undergoing treatment with conventional DMARDs, subject is on a stable dose for at least 12 weeks prior to the Baseline Visit (Visit 2).
8. For subjects who have received treatment with biological DMARDs, subject has the required washout (normalization) period prior to the Baseline Visit (Visit 2). The washout (normalization) period for biological DMARDs is as follows:
   a. Anakinra - 1 week
   b. Etanercept, rilonacept - 4 weeks
   c. Adalimumab, certolizumab, infliximab, golimumab, abatacept, tocilizumab and canakinumab - 8 weeks
   d. Rituximab - 36 weeks
9. Females of childbearing potential, and males with female partners of childbearing potential, who participate in the study agree to use a highly effective method of contraception throughout the study and for 28 days following the last dose of study drug. A highly effective method of birth control is defined as one that results in a low failure rate (ie, <1% per year) when used consistently and correctly, such as oral/injectable/inserted/implanted/transdermal contraceptives, condom with diaphragm, condom with spermicide, diaphragm with spermicide, intrauterine hormone-releasing system or intrauterine device (IUD), or sexual abstinence. Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (12 consecutive months of spontaneous amenorrhea and age ≥51 years).
10. Subject has provided written informed consent for this study.
11. Subject is willing and able to comply with the protocol.

### Exclusion Criteria

The presence of any of the following criteria excludes a subject from the study:
1. Subject is, in the opinion of the investigator, mentally or legally incapacitated, or has significant emotional problems at the time of the Screening Visit (Visit 1) that could interfere with the subject’s participation or cooperation with the conduct of study evaluations.
2. Subject has a chronic severe or uncontrolled medical disorder that might confound the results of safety assessments conducted in the study.
3. Subject has another serious chronic-inflammatory disease.
4. Subject has a relevant, active infection or another disease, which entails a tendency towards infection.
5. Subject has active macrophage activation syndrome.
6. Subject has a history of unresolved latent tuberculosis.
7. Subject has the following abnormal values:
   a. Serum creatinine concentration >1.5 mg/dl.
   b. Hemoglobin ≤ 10 g/dl, neutrophils ≤1,500/µl and/or thrombocytes ≤75,000/µl.
8. Subject has a history of neoplasia with the exception of a curatively treated non-melanoma skin tumor or carcinoma of the cervix treated in situ without any indication of recurrence within the last 10 years.
9. Subject has received a vaccination with a live vaccine within 12 weeks prior to the Baseline Visit (Visit 2).
10. Subject has used an investigational product or been enrolled in a clinical study including vaccines within 30 days of the Screening Visit (Visit 1).
11. Subject has known or suspected intolerance or hypersensitivity to the investigational product(s), closely related compounds, or any ingredients of the investigational product.

### Screen Failures

Subjects who fail inclusion and/or exclusion criteria may be rescreened for the study with the prior approval of the Medical Monitor. In the event of a rescreening, the first screening visit will be entered into the electronic case report form (eCRF) as the Screening Visit and the repeat assessments entered into the eCRF as an unscheduled visit.

### Premature Subject Withdrawal

All subjects will be advised that they are free to withdraw from participation in this study at any time, for any reason, and without prejudice. Every reasonable attempt should be made by the investigator to keep subjects in the study; however, subjects must be withdrawn from the study if they withdraw consent to participate. Investigators must attempt to contact subjects who fail to attend scheduled visits by telephone or other means to exclude the possibility of an AE being the cause of withdrawal. Should this be the cause, the AE must be documented, reported, and followed.

Subjects can decline to continue receiving study drug at any time during the study. If this occurs, the investigator is to discuss with the subject the completion of the Follow-up / Early Termination Visit 4 weeks following the last dose of study drug.

Withdrawal of consent for a study means the subject does not wish to receive further protocol-required treatment or procedures, and the subject does not wish to or is unable to continue further study participation. Subject data up to withdrawal of consent will be included in the analysis of the study, and where permitted, publicly available data can be included after withdrawal of consent. If withdrawal of consent occurs before 4 weeks have elapsed since the last dose of study drug, the investigator is to discuss with the subject the procedure for the Safety Follow-up Telephone Call to occur 4 weeks after the last dose of study drug. Subjects can decline to participate in the telephone call.

The Sponsor reserves the right to request the withdrawal of a subject due to protocol deviations or other reasons.

The investigator also has the right to withdraw subjects from the study at any time for anyreason. If a subject is withdrawn before completing the study, the subject should be followed-up as instructed in the Schedule of Assessments (Table 1). The reason for withdrawal must be determined by the investigator and recorded in the subject’s medical record and in the electronic case report form (eCRF). If a subject is withdrawn for more than 1 reason, each reason should be documented in the source document and the most clinically relevant reason should be entered in the eCRF.

Reasons for discontinuation include but are not limited to:
Lack of effect, including worsening of symptoms, disease progression, or lack of improvement
Adverse event
Death
Non-compliance with study procedure/protocol
Decision by investigator
Decision by Sponsor
Withdrawal of consent by subject
Lost to follow-up
Other (specify). For example, pregnancy.

### Subject Replacement Criteria

Subjects who withdraw from the study for reasons other than drug toxicity may be replaced with the prior approval of the Medical Monitor.

TABLE 1

Schedule of Assessments

| | Screening Visit^{a} | Baseline Visit^{b} | Treatment Period | | | | Follow-up/ Early Termination° |
|---|---|---|---|---|---|---|---|
| Visit | 1 | 2 | 3 | 4 | 5 | 6 | 7 |
| Assessment Week | -4 | 0 | 1 | 2 | 4 | 8 | 12 |
| Visit Window | | | ±2 days | ±2 days | ±2 days | ±2 days | ±2 days |
| Informed Consent ^{c, p} | X | | | | | | |
| Inclusion/Exclusion Review ^{p, q} | X | X | | | | | |
| Demographics ^{p, q} | X | | | | | | |
| Medical and Medication History ^{p, q, r} | X | | | | | | |
| Concomitant Medications ^{p, q} | X | X | X | X | X | X | X |
| Adverse Event Monitoring ^{n, p, q} | X | X | X | X | X | X | X |
| Vital Signs ^{d, p, q} | X | X | X | X | X | X | X |
| Physical Exam ^{q} | X | X | X | X | X | X | X |
| Height ^{q} | X | | | | | | |
| 12-lead ECG^{q} | | X | | | | | |
| Patient global assessment of disease activity VAS ^{f, q} | | X | X | X | X | X | X |
| Subject self-report of fever ^{e, p, q} | X | X | X | X | X | X | X |
| Physician global assessment of disease activity VAS ^{f, p} | | X | X | X | X | X | X |
| Modified Pouchot Score ^{p, q} | | X | X | X | X | X | X |
| Tender and Swollen Joint Count (DAS28-CRP) ^{q} | | X | X | X | X | X | X |
| Hematology, Clinical Chemistry, Ferritin, CRP, IL-18 and IL-18BP ^{g, q} | X | X | X | X | X | X | X |
| ESR ^{h, q} | | X | X | X | X | X | X |
| PK^{i, q} | | X | X | X | X | X | X |
| ADA^{j, q} | | X | | X | | X | X |
| Urinalysis ^{k, q} | | X | X | X | X | X | X |
| Serum pregnancy test ^{l, q} | X | | | | | | |
| Urine pregnancy test ^{g, l, q} | | X | | | X | X | X |
| Study Drug Administration ^{m, n, q} | | X | | | X | X | |

a. Local laboratory results (creatinine, hemoglobin, neutrophils, thrombocytes, and serum pregnancy) may be utilized to expedite enrollment; however, central laboratory specimens must also be collected as part of the screening procedures.
b. Subjects must meet the required normalization (washout) period for biological DMARDs prior to the Baseline Visit (Visit 2). The normalization period for DMARDs is as follows: Anakinra - 1 week; Etanercept, rilonacept - 4 weeks; Adalimumab, certolizumab, infliximab, golimumab, abatacept, tocilizumab, canakinumab - 8 weeks; Rituximab - 36 weeks.
c. Re-consent is required if the 28-day Screening or Re-screening periods have lapsed.
d. Includes pulse, blood pressure, respiratory rate, body temperature, and body weight. Body temperature will be measured by the subject 4 times daily, prior to taking any antipyretics, until fever is resolved (not more than 37.5° C.) for 48 hours. Blood pressure to be measured on the same arm (preferentially the left arm) after the subject has been in a supine or sitting position for 5 minutes. Pulse may be recorded simultaneously with blood pressure measurements. Body weight to be measured without shoes or jacket.
e. Subject will be asked to self-report their maximum temperature and corresponding date within the last 5 days of the Screening and Baseline visits. At subsequent visits the subject will be asked to self-report their maximum temperature within the last 48 hours.
f. Patient Global Assessment to be performed prior to the Physician Global Assessment.
g. If the Baseline visit occurs within 48 hours of the Screening visit, labs tests occurring for both visits will be collected only once at Screening, except for the IL-18/IL-18BP sample which will be collected at both visits. For pregnancy testing, only the serum test will be collected; the urine test does not need to be performed.
h. ESR kits will be provided for the site to run the test locally.
i. PK sampling will be performed at Baseline at 2 hours post-dose; at Weeks 1, 2, and 12 at any time during the visit; and at Weeks 4 and 8 at predose and 2 hours post-dose.
j. ADA collection to be performed predose.
k. Urinalysis to include pH, specific gravity, dipstick determinations of protein, blood, glucose and ketones; microscopic if blood or protein 2+ or higher.
l. For females of childbearing potential. A subject is not considered to be of childbearing potential if she is postmenopausal (12 consecutive months of spontaneous amenorrhea and age ≥51 years); surgically sterile (having undergone one of the following surgeries: hysterectomy, bilateral tubal ligation or occlusion, bilateral oophorectomy, or bilateral salpingectomy); and at where at least 6 weeks has passed since sterilization. Positive urine pregnancy test results should have a local serum pregnancy test performed as confirmation.
m. Investigational drug to be administered after all assessments are completed.
n. Subjects should be observed for signs and symptoms of hypersensitive or allergic reactions, with monitoring of vital signs as needed, from administration of Antibody A through the 2-hour post-dose PK sample collection.
o. Subjects who withdraw from the study less than 28 days from the last dose, and who decline to return to the site for the early termination visit, may have relevant early termination procedures listed in the eCRF performed via a Safety Follow-up Telephone Call.
p. Due to the COVID-19 pandemic, if the subject cannot be present in person, these activities may be partially or fully conducted via a telemedicine visit (video) or phone call. Site procedures put in place for conducting a telemedicine/phone visit must maintain a subject’s privacy. The site’s documents must record the date of the telemedicine visit. In the event remote informed consent procedures are utilized, acceptable processes such as those described in guidances must be utilized. Eg, FDA Guidance on Conduct of Clinical Trials of Medical Products during COVID-19 Public Health Emergency, and EMA’s Guidance on the Management of Clinical Trials During the Covid-19 (Coronavirus) Pandemic.
q. Due to the COVID-19 pandemic, if the subject cannot be present in person, these activities may be partially or fully conducted through a home healthcare visit. The investigator will ensure that home healthcare providers performing the tender/swollen joint count are trained in the conduct of the assessment.
r. Medication History to include any prior or current AOSD medications with the information from the last treatment exposure, and all medications taken within 28 days of screening through 28 days post last dose of study drug.
Abbreviations: ADA = anti-drug antibody; CRP = C-reactive protein; DAS28-CRP = Disease Activity Score-28 for rheumatoid arthritis with CRP; DMARD = disease-modifying anti-rheumatic drugs; ECG = electrocardiogram; ESR = erythrocyte sedimentation rate; IL-18 = interleukin-18;
IL-18BP = interleukin-18 binding protein; PK = pharmacokinetic; VAS = Visual Analog Scale.

### Example 1.3 - Treatments

### Identification of Investigational Product, Dose and Mode of Administration

Antibody A is the investigational product that will be and was used in this study. It was in the form of lyophilized powder in a single-use glass vial. The dose was 7 mg/kg (maximum 500 mg) for the first cohort of 6 subjects, and a predefined algorithm will determine the dosing of the second cohort of 6 subjects. The frequency of dosing was and is once every 4 weeks during the treatment period. The mode of administration was and is intravenous.

### Treatments Administered

Antibody A will be and was administered at a dose of 7 mg/kg (maximum 500 mg) IV every 4 weeks to the first cohort of 6 subjects. Safety will be and was assessed by an SRC for these 6 subjects. Twenty-eight days after the first cohort’s 6th subject has completed his/her first dose of Antibody A, if the safety is acceptable per a predefined algorithm, the second cohort of 6 subjects will be dosed with 14 mg/kg (maximum 1000 mg) IV. Per the predefined algorithm, the SRC may decide to expand the number of subjects in the first cohort before proceeding with the dose escalation in the second cohort of subjects. If the safety of 7 mg/kg is not acceptable, dosing will be decreased to 4 mg/kg (maximum 300 mg) per the predefined algorithm.

Antibody A will be and was administered via a slow IV infusion (over a minimum of 60 minutes) to reduce the risk of infusion reactions. Appropriate drugs and medical equipment to treat acute anaphylactic must be immediately available, and study personnel must be trained to recognize and treat anaphylaxis. Subjects should be observed for signs and symptoms of hypersensitive or allergic reactions, with monitoring of vital signs as needed, from administration of Antibody A through the 2-hour post-dose PK sample collection.

### Blinding and Unblinding Treatment Assignment

This is an open-label study.

### Selection of Doses in the Study

The doses of 4, 7, and 14 mg/kg, administered every 4 weeks, are contemplated or were used in this study.

### Dose Adjustment Criteria

Antibody A will be and was administered at a dose of 7 mg/kg (500 mg maximum) IV every 4 weeks to the first cohort of 6 subjects. Based on the review by the Safety Review Committee, if the safety is acceptable per a predefined algorithm, the second cohort of 6 subjects will be dosed with 14 mg/kg (maximum 1000 mg) IV. Per the predefined algorithm, the SRC may decide to expand the number of subjects in the first cohort before proceeding with the dose escalation in the second cohort of subjects. If not acceptable, dosing will be decreased to 4 mg/kg (maximum 300 mg) per the predefined algorithm.

### Prior Therapies

Prior therapies includes all treatments received within 28 days of the date of first dose of investigational product.

In addition, all AOSD treatments received at any time, whether during or prior to the 28-day window noted above, should be entered with the information from the last treatment exposure. Eg, a biological DMARD that has been normalized (washed out) 8 weeks prior to the Baseline Visit (Week 0) should have the last date and dose received entered in the appropriate eCRF page.

### Concomitant Therapies

Concomitant therapies refer to all therapies taken from Screening and for 4 weeks after the last dose of study drug or last visit, whichever is later. Concomitant therapy information must be recorded on the appropriate eCRF page.

Subjects that enter the study with a stable dose of glucocorticoids are expected to maintain the dose for the study duration. If a change is needed to reduce the dose, it should be done under best medical practice after the Week 4 visit / 28 days on study drug.

### Permitted Therapies

Patients are treated for all intercurrent medical conditions at the discretion of the investigator in accordance with community standards of medical care. Subjects receive treatments for palliation of symptoms associated with AOSD.

Subject’s vaccination status should be reviewed in accordance with current immunization guidelines prior to study start. It is recommended that vaccination be avoided within 4 weeks of initiating treatment with Antibody A.

### Prohibited Therapies

Subjects must meet the 48-hour stable dose requirement prior to the Baseline Visit (Visit 2) if a subject is undergoing treatment with NSAIDs and/or glucocorticoids.

Subjects must meet the required normalization (washout) period for biological DMARDs prior to the Baseline Visit (Visit 2). The normalization period for DMARDs is as follows: a) anakinra (1 week); b) etanercept, rilonacept (4 weeks); c) adalimumab, certolizumab, infliximab, golimumab, abatacept, tocilizumab, and canakinumab (8 weeks); and d) rituximab (36 weeks).

Any new therapy, other than Antibody A, designed to treat the subject’s AOSD is prohibited during the study. This includes biologics (eg, anti-IL-1, anti-IL-6, anti-TNFα) as well as the initiation or increase of any glucocorticoid treatment.

During the study, new initiation of investigational compounds is prohibited. Vaccines that are considered by the investigator during the clinical trial should be discussed with the medical monitor prior to administration.

At the discretion of the Sponsor, subjects receiving excluded therapies during the study may be ineligible for continuation in the study.

### Treatment After End of Study

Following discontinuation from the study, subjects may be treated at the discretion of the investigator.

### Example 1.4 - Study Procedures

### Study Duration

The sequence and maximum duration of the study periods will be as follows. The screening period will be up to 28 days. The treatment period will be 8 weeks. The follow-up period will be 4 weeks after the last dose of the investigational product or last visit, whichever is later. The maximum study duration for each subject is up to 16 weeks. The maximum treatment duration for each subject is 8 weeks.

### Safety Assessments

Safety and tolerability assessments will include the frequency and severity of AEs as well as the evaluation of changes in clinical laboratory values and vital signs.

### Clinical Laboratory Tests to be Performed

Samples for the following clinical laboratory tests will be collected at the time points specified in the Schedule of Assessments (Table 1).

Hematology tests include Hemoglobin, hematocrit, platelet count (or estimate), and white blood cell count including differential.

Serum chemistry tests include albumin, total bilirubin, total protein, calcium, alkaline phosphatase, alanine aminotransferase, aspartate aminotransferase, lactate dehydrogenase, blood urea nitrogen/urea, creatinine, magnesium, glucose, sodium, potassium, chloride, bicarbonate, phosphorous, and uric acid.

A serum/urine pregnancy test will be administered for females of childbearing potential.

Urinalysis tests include pH, specific gravity, dipstick determinations of protein, blood, glucose and ketones; and microscopic if blood or protein 2+ or higher.

Laboratory specimens will be collected and shipped to a central laboratory facility for processing by the appropriate laboratory. Local laboratory results (creatinine, hemoglobin, neutrophils, thrombocytes, and serum pregnancy) may be utilized to expedite enrollment; however, central laboratory specimens must also be collected as part of the screening procedures.

### Sampled Blood Volume

The sampled blood volume is shown in Table 2.

TABLE 2

Sampled Blood Volume per Subject

| | | | |
|---|---|---|---|
| Type | Volume per sample | Sample number | Total |
| Chemistry, Efficacy (Ferritin and CRP), Serum pregnancy | 3.5 mL | 7 | 24.5 mL |
| Efficacy (ESR only) | 2 mL | 6 | 12 mL |
| Hematology | 2 mL | 7 | 14 mL |
| Pharmacokinetics | 2 mL | 8 | 16 mL |
| Pharmacodynamics markers (IL-18, IL-18BP) | 8.5 mL | 7 | 59.5 mL |
| Anti-drug antibody | 2 mL | 4 | 8 mL |
| Total (up to) | | | 134.0 mL |

These parameters exclude rescreening laboratory samples, locally run laboratory tests performed to expedite enrollment or to monitor potential adverse events, confirmatory pregnancy tests, or for any unscheduled visits. The total collected volume may be less than the above in cases where the Baseline visit occurs with 48 hours of the Screening visit.
Abbreviations: CRP = C-reactive protein; ESR = erythrocyte sedimentation rate; IL-18 = interleukin-18; IL-18BP = interleukin 18 binding protein.

### Evaluation of Laboratory Values

The normal ranges of values for the laboratory assessments in this study will be provided by the applicable laboratory facility. They will be regarded as the reference ranges on which decisions will be made for the specific site.

If a laboratory value is out of the reference range, it is not necessarily clinically relevant. The investigator must evaluate the out-of-range values and record his/her assessment of the clinical relevance in the subject’s source documentation. Abnormal laboratory values may be considered AEs if they are clinically significant, if they require an intervention, or if they change the administration of study drug.

All laboratory values which, in the investigator’s opinion, show clinically relevant or pathological changes during or after termination of the treatment are to be discussed with the Medical Monitor, as necessary, and reported as AEs and followed.

### Clinical Examination: Vital Signs

Blood pressure, pulse rate, respiratory rate, temperature, and body weight will be measured at times specified in the Schedule of Assessments (Table 1). Additional blood pressure and pulse rate measurements may be performed, as determined by the investigator, to ensure appropriate monitoring of subject safety and accurate recording of vital sign measurements. Any changes from baseline which are deemed clinically significant by the investigator are to be recorded as an AE and followed.

### Clinical Examination: Electrocardiogram

A standard 12-lead ECG will be performed after the subject has been supine for approximately 5 minutes. All ECG recordings will be identified with the subject number, date, and time of the recording and a copy will be included with the subject’s source documentation.

Twelve-lead ECGs will be performed locally at Baseline and then as required (Table 1). All ECG values which, in the investigator’s opinion, show clinically relevant or pathological changes during or after termination of the treatment are to be discussed with the Medical Monitor and reported as AEs and followed.

### Clinical Examination: Physical Examination

A physical examination will be performed at the Screening Visit before potential exposure to the investigational product and at subsequent study visits (Table 1). Any clinically significant physical examination findings post-screening are to be reported as AEs and followed.

### Clinical Examination: Adverse Events

The investigator is responsible for the detection and documentation of events meeting the criteria and definition of an AE or SAE described previously. At each visit, the subject will be allowed time to spontaneously report any issues since the last visit or evaluation.

Any clinically relevant observations made during the visit will also be considered AEs.

### Efficacy

The efficacy of Antibody A will be determined by fever resolution, CRP, ferritin, ESR, Physician and Patient Global Assessments using VAS, modified Pouchot score, and DAS28CRP.

Fever resolution will be determined by measuring body temperature at study visits and collecting subject reports of fevers experienced within the last 5 days prior to the Screening and Baseline Visits, and within the last 48 hours at subsequent visits.

Body temperature, CRP, ferritin, and ESR will be measured at the times specified in the Schedule of Assessments (Table 1).

The Patient Global Assessment of Disease Activity (PtGA) will be measured at the times specified in the Schedule of Assessments (Table 1), using 100 mm VAS ranging from no evidence of disease (0 mm) to very severe disease activity (100 mm).

The Physician Global Assessment of Disease Activity (PhGA) will be measured at the times specified in the Schedule of Assessments (Table 1), using 100 mm VAS ranging from no evidence of disease (0 mm) to very severe disease activity (100 mm). To enhance objectivity, the physician must not be aware of the specific PtGA, when performing his/her own assessment on that patient.

The modified Pouchot score will be measured at the times specified in the Schedule of Assessments (Table 1). For each of the 12 clinical conditions listed, a score count of 1 is assigned for any that are present at the time of assessment (Rau et al. (2010) J Rheumatol. 37(11):2369-2376): fever; evanescent rash; pharyngitis; arthritis; myalgia; pleuritis; pericarditis; pneumonitis; lymphadenopathy; hepatomegaly or elevated liver enzymes; Leukocyte count > 15,000/µl; Ferritin > 3000 µg/l. A total score of > 4 is considered to indicate active disease.

DAS28-CRP will be measured at the times specified in the Schedule of Assessments (Table 1). To calculate the DAS28-CRP, information about the following disease variables will be collected: the number of swollen joints and tender joints using 28-joint count (TJC28 and SJC28); PtGA measured on a VAS of 100 mm; and CRP measured in mg/L.

Using this data, the DAS28-CRP will be calculated using the following formula:

$$\begin{matrix}0.56 * \text{sqrt} \left(\text{TJC28}\right) + 0.28 * \text{sqrt} \left(\text{SJC28}\right) + 0.36 * \text{ln} \left(\text{CRP+1}\right) \\ + 0.014 * \text{PtGA + 0} \text{.96}\end{matrix}$$

Swollen joints, tender joints and PtGA assessment will be collected and CRP will be used to determine DAS28-CRP scores.

The DAS28 score ranges from 0 to 10, with higher scores indicating a more severe current activity of the disease. The DAS28 score will also be categorized into disease activity states according to the cut-off values provided in Table 3.

TABLE 3

Definition of Disease Activity According to DAS28

| Disease activity state | Cut-off value |
|---|---|
| High disease activity | >5.1 |
| Moderate disease activity | ≥3.2 to ≤5.1 |
| Low disease activity | >2.6 to <3.2 |
| Remission | ≤2.6 |

### Pharmacokinetics

Blood draws for PK analysis will be performed in accordance with Table 1. Serum samples will be analyzed via validated methods for serum Antibody A concentrations.

### Pharmacodynamics

PD will be measured by levels of IL-18 and IL-18BP, obtained at various time points during study participation (Table 1).

### Immunogenicity

Blood draws for immunogenicity analysis will be performed in accordance with Table 1. Details of immunogenicity sample collection and processing can be found in the site and/or study laboratory manual. Serum samples will be analyzed via validated methods for ADA determination.

### Example 1.5 - Adverse Events

### Adverse Event Collection

An AE is defined as any untoward medical occurrence in a patient or clinical investigation subject administered a pharmaceutical product that does not necessarily have a causal relationship with the product. An AE can therefore be any unfavorable and unintended sign (including a new, clinically important abnormal laboratory finding), symptom, or disease, temporally associated with the product, whether or not related to the product. An AE will be considered treatment-emergent if it occurs after the first dose of investigational product (including the duration of infusion) and within 28 days of a subject’s last dose of investigational product.

All AEs are collected from the time the informed consent is signed through the follow-up period (Table 1). This includes events occurring during the screening phase of the study, regardless of whether investigational product is administered. Where possible, a diagnosis rather than a list of symptoms should be recorded. If a diagnosis has not been made, then each symptom should be listed individually. All AEs should be captured on the appropriate AE pages in the eCRF and in source documents.

All AEs must be followed to closure, regardless of whether the subject is still participating in the study. Closure indicates that an outcome is reached, stabilization is achieved (ie, the investigator does not expect any further improvement or worsening of the event), or the event is otherwise explained. When appropriate, medical tests and examinations are performed so that resolution of an event(s) can be documented.

Lack of effect, including worsening of symptoms, disease progression, or lack of improvement, should not be recorded as an AE unless it meets the definition of the criteria for an SAE. Signs and symptoms of AOSD that are tracked as part of the efficacy evaluations, eg conditions listed in the modified Pouchot score, should not be captured as AEs unless they meet the definition of an SAE.

AE that changes in severity over time should be recorded in the eCRF once at the highest severity with two exceptions. The first exception is worsening of non-AOSD related pretreatment events after initiation of investigational product must be recorded as new AEs. For example, if the subject experiences mild, intermittent headaches prior to dosing with investigational product; however, the headache intensity increases to moderate after the first dose of investigational product, a new AE of moderate intermittent headaches is to be recorded in the source documents and eCRF. The second exception is an AE which begins as a non-serious event, which later meets the definition of an SAE, should be entered once for the non-serious portion of the AE, and then be re-recorded as a new event with the start date the day it became serious.

### Severity of Adverse Events

The medical assessment of clinical severity of an AE will be determined using the definitions outlined in Common Terminology Criteria for Adverse Events (CTCAE), Version 5.0 (Published Nov. 27, 2017 by the US Department of Health and Human Services, National Institutes of Health, National Cancer Institute). Grade 1 is defined as Mild; asymptomatic or mild symptoms; or clinical or diagnostic observations only; or intervention not indicated. Grade 2 is defined as Moderate; or minimal, local or non-invasive intervention indicated; or limiting age-appropriate instrumental activities of daily living (ADL). Grade 3 is defined as Severe or medically significant but not immediately life-threatening; or hospitalization or prolongation of hospitalization indicated; or disabling; or limiting self-care ADL. Grade 4 is defined as Life-threatening consequences; or urgent intervention indicated. Grade 5 is death related to AE.

The above grading guidelines should be used whenever possible. For AEs that cannot be graded by the use of CTCAE, the severity should be graded using mild (Grade 1), moderate (Grade 2), severe (Grade 3), life threatening (Grade 4), and fatal (Grade 5).

Please refer to the above-referenced CTCAE document for full description of CTCAE terms and instrumental and self-care ADLs. It is important to distinguish between severe AEs and SAEs. Severity is a classification of intensity whereas an SAE is an AE that meets serious criteria.

### Relationship Categorization

A physician investigator must make the assessment of relationship to investigational product for each AE. The investigator should decide whether, in his or her medical judgment, there is a reasonable possibility that the event may have been caused by the investigational product. If there is no valid reason for suggesting a relationship, then the AE should be classified as “not related”. Otherwise, the AE should be categorized per the guidelines below. The causality assessment must be documented in the source document and the eCRF (Table 4).

TABLE 4

Assessment of Relationship to Investigational Product

| | |
|---|---|
| Relationship | Description |
| Not Related | Exposure to investigational product has not occurred. |
| | OR |
| | The administration of investigational product and the occurrence of the AE are not reasonably related in time. |
| | OR |
| | The AE is considered likely to be related to an etiology other than the use of the investigational product, that is, there are no facts/evidence or arguments to suggest a causal relationship to the investigational product. |
| Possibly Related | The administration of the investigational product and the occurrence of the AE are reasonably related in time. |
| | AND |
| | The AE could not be explained equally well by factors or causes other than exposure toinvestigational product. |
| Probably Related | The administration of the investigational product and the occurrence of the AE are reasonably related in time. |
| | AND |
| | The AE is more likely explained by exposure to investigational product than by otherfactors or causes. |

AE = adverse event.

### Outcome at the Time of Last Observation

The outcome at the time of last observation will be classified as: recovered/resolved; recovered/resolved with sequelae; recovering/resolving; not recovered/not resolved; fatal; or unknown.

### Reporting of Serious Adverse Events

Initial and follow-up SAE reports must be completed by the investigator or designee and sent to the contract research organization (CRO) within 24 hours of the first awareness of a SAE. The investigator or designee must complete, sign and date the appropriate SAE form and verify the accuracy of the information against corresponding source documents. This information is to be sent to the CRO Pharmacovigilance Department.

### Serious Adverse Event Definition

An SAE is any untoward medical occurrence, whether considered to be related to investigational product or not, that at any dose: results in death; is life-threatening; requires inpatient hospitalization or prolongation of existing hospitalization; results in persistent or significant disability/incapacity; is a congenital anomaly; or is an important medical event.

Note that the term “life-threatening” in the definition of “serious” refers to an event in which the subject was at risk of death at the time of the event; it does not refer to an event which hypothetically might have caused death if it were more severe.

Note that inpatient hospitalization is defined as 24 hours in a hospital or an overnight stay. An elective hospital admission to treat a condition present before exposure to the test drug, or a hospital admission for a diagnostic evaluation of an AE, does not qualify the condition or event as an SAE. Further, an overnight stay in the hospital that is only due to transportation, organization, or accommodation problems and without medical background does not need to be considered an SAE.

Note that a congenital anomaly in an infant born to a mother who was exposed to the investigational product during pregnancy is an SAE. However, a newly diagnosed pregnancy in a subject that has received an investigational product is not considered an SAE unless it is suspected that the investigational product interacted with a contraceptive method and led to the pregnancy.

Note that medical and scientific judgment should be exercised in deciding whether it is appropriate to consider other situations serious, such as important medical events that may not be immediately life-threatening or result in death or hospitalization but may jeopardize the subject or may require intervention to prevent one of the other outcomes listed in the definition above. Examples of such events are intensive treatment in an emergency room or at home for allergic bronchospasm, blood dyscrasias or convulsions that do not result in hospitalization, or development of drug dependency or drug abuse.

### Serious Adverse Event Collection Time Frame

All SAEs, regardless of the relationship to study, are collected from the time the subject signs the informed consent until the subject’s last visit (office or telephone contact). The investigator or designee must report all SAEs promptly to the CRO within 24 hours of first becoming aware of the event.

Any SAE(s), regardless of relationship to study drug, that occurred during the study, but which is not discovered by the study site until after study has completed must be reported to the CRO within 24 hours of the first awareness of the event.

### Serious Adverse Event Onset and Resolution Dates

The onset date of the SAE is defined as the date the event meets serious criteria. An AE which begins as a non-serious event, which later meets the definition of an SAE, should be entered once for the non-serious portion of the AE, and then be re-recorded as a new event with the start date the day it became serious.

The resolution date is the date an outcome is reached, stabilization is achieved (ie, the investigator does not expect any further improvement or worsening of the event), or the event is otherwise explained.

### Fatal Outcome

Fatal should only be designated as an outcome when the AE results in death. If more than 1 AE is possibly related to the subject’s death, the outcome of death should be indicated for each such AE.

Any AE that results in the subject’s death must have fatal checked as an outcome with the date of death recorded as the resolution date. AEs resulting in death must be reported within 24 hours as a SAE, if not already reported as such.

For other AEs, ongoing at the time of death that did not contribute to the subject’s death, the outcome should be considered not resolved, without a resolution date recorded.

### Adverse Events of Special Interest

The following events will be considered as AEs of special interest during this study: immunosuppression (decreased white counts, thrombocytopenia, anemia).

### Pregnancy

All females of childbearing potential, and males with female partners of childbearing potential, who participate in the study should be counseled on the need to utilize a highly effective method of birth control throughout the study and for 28 days following the last dose of study drug, and on the importance of avoiding pregnancy during study participation. A highly effective method of birth control is defined as one that results in a low failure rate (ie, <1% per year) when used consistently and correctly, such as oral/injectable/inserted/implanted/ transdermal contraceptives, condom with diaphragm, condom with spermicide, diaphragm with spermicide, intrauterine hormone-releasing system or intrauterine device (IUD), or sexual abstinence. Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (12 consecutive months of spontaneous amenorrhea and age ≥51 years).

Females and males with female partners should be instructed to contact the investigator or study staff immediately if pregnancy occurs or is suspected.

Pregnancy testing will be conducted on every female as per the schedule of assessments (Table 1). A female who is found to be pregnant at Screening will be excluded from the study and considered to be a screening failure. A female who is found to be pregnant after receiving investigational product is required to be discontinued from the study and the end of study visit assessments performed as soon as possible after learning of the pregnancy.

The investigator must report the pregnancy of any female (study participant or female partner of male study participant) who becomes pregnant during investigational product treatment or within 28 days of discontinuing the investigational product (permission must be obtained from the pregnant female partner of a male patient to follow the pregnancy to conclusion and report the results). The pregnancy must be reported within 24 hours of learning of the pregnancy to the CRO using the Pregnancy Data Collection Form via the same fax and email address as for SAE reporting. The investigator should contact the designated individual(s) who receive pregnancy notification and record information related to the pregnancy on the Pregnancy Form/other designated form provided by the Sponsor or its designee.

The investigator is also responsible for following the pregnancy until delivery or termination. These findings must be reported on the Pregnancy Data Collection Form and forwarded to the designated individual(s). The event meets the SAE criterion only if it results in a spontaneous abortion or a congenital anomaly.

### Reporting to Regulatory Agency, Institutional Review Board/Ethics Committee and Site

The Sponsor or its designee is responsible for notifying the relevant regulatory authorities and if applicable, US central institutional review board (IRB) of related, unexpected SAEs.

In addition, the Sponsor or its designee is responsible for notifying active sites of all related, unexpected SAEs occurring during all interventional studies across the development program.

The investigator is responsible for notifying the local IRB, local ethics committee (EC), or the relevant local regulatory authority of all SAEs that occur at his/her site, as required.

### Example 1.6 - Overdose and Medication Error

Overdose or medication error of investigational product, defined below (Table 5), must be reported to the Sponsor using the SAE reporting procedures outlined above whether or not they result in an AE/SAE. The 24-hour reporting period for SAEs does not apply to overdose or medication error event(s) unless the overdose or medication error event results in an SAE.

TABLE 5

Definition of Overdose and Medication Error

| | |
|---|---|
| Category | Definition |
| Overdose | Intentional or unintentional intake of a dose of investigational product exceeding more than 10% of the dose prescribed to the subject as part of the clinical trial. |
| Medication Error | Error made in prescribing, dispensing, administration and/or use of investigational product. |
| | Medication errors are reportable to the Sponsor or its designee if it involves: |
| | Dispensing, administration, and/or use of an unassigned treatment (for example, incorrect investigational product kit used by subject). |
| | Dispensing, administration and/or use of expired investigational product. |

Missing doses are not considered a medication error event and do not need reporting.
Note that an overdose or medication error event can meet one or both of the above categories.

### Example 1.7 - Safety Review Committee

A Safety Review Committee (SRC) will be involved in the conduct of this study. The SRC will be comprised of representatives from the Sponsor and the study sites, including 1 medically qualified member from each participating site. The SRC has the responsibility for monitoring the clinical study’s progress and the safety of the participating subjects.

Approximately 12 subjects are planned to be enrolled in 2 cohorts. The first cohort of 6 subjects will receive and did receive Antibody A at 7 mg/kg dose (maximum 500 mg) IV at Baseline, Week 4, and Week 8. Twenty-eight days after the first cohort’s 6th subject has completed his/her first dose of Antibody A, safety will be assessed by the SRC for the first cohort, specifically: AEs, SAEs, and laboratory values.

If there is >1 SAE in the first cohort that is attributed to Antibody A, the second cohort of 6 subjects will be dosed at 4 mg/kg (maximum 300 mg) IV at Baseline, Week 4, and Week 8.

If there is 1 SAE in the first cohort that is attributed to Antibody A, the SRC will assess the safety information and determine whether to expand the number of subjects in the first cohort or to proceed with the dose escalation in the second cohort of subjects.

If there are no SAEs attributed to Antibody A, the SRC will determine whether to proceed with the dose escalation in the second cohort of 6 subjects. The escalated dose regimen will be 14 mg/kg (maximum 1000 mg) IV at Baseline, Week 4, and Week 8. Following the dose escalation, if the second cohort experiences 1 SAE attributed to Antibody A, the SRC will assess the safety information and determine whether to continue the subjects at 14 mg/kg (maximum 1000 mg) dose or to drop down to the prior 7 mg/kg (500 mg maximum) dose. Following the dose escalation, if the second cohort experiences > 1 SAE attributed to Antibody A, subjects will be dropped down to the prior 7 mg/kg (500 mg maximum) dose.

### Example 1.8 - Statistics

Approximately 12 subjects are planned to be enrolled. Antibody A will be investigated in patients with AOSD for the first time in this study. Hence, the sample size is based on feasibility and not on hypothesis testing.

This study will have the following populations of interest: The Enrolled Population includes all subjects who are enrolled; and the Safety Population includes all subjects who are enrolled and receive at least one treatment administration during this trial.

This section presents a summary of the planned statistical analyses. Additional details regarding data handling, analytical methods, and presentation of results will be provided in a Statistical Analysis Plan (SAP) for this study. The SAP will be finalized prior to database lock.

All efficacy, safety, PK, and PD variables will be summarized using descriptive statistics. Descriptive statistics for continuous data will include number of subjects (n), mean, standard deviation (SD), median, minimum, and maximum. Summaries of change from baseline variables will include only subjects who have both a baseline value and corresponding value at the time point of interest. For all variables, baseline will be defined as the last assessment prior to the first dose of study drug. Descriptive statistics for categorical data will include frequency and percentage. Where appropriate, descriptive statistics may be presented with 95% confidence intervals. All descriptive summaries will be produced by Antibody A dose level and will include an overall category.

The disposition of all subjects enrolled in this study will be summarized by dose level and completion/discontinuation status. Subjects who discontinue the study prematurely will be summarized by dose level and reason for discontinuation. The number of subjects in each analysis set will also be summarized by dose level.

All subject data will be reviewed for the occurrence of protocol deviations. Prior to database lock, all protocol deviations will be reviewed and classified with respect to the potential to influence experimental outcomes.

The analysis of demographic and baseline data will be performed for the Safety Population. Demographic variables include age, gender, race, ethnicity, height, weight, and body mass index.

Demographics and other baseline characteristics will be summarized by dose level using descriptive statistics.

Medications will be coded using the World Health Organization Drug Dictionary. Prior and concomitant medications will be summarized by dose level.

Exposure to investigational product will be summarized by dose level using descriptive statistics. Subjects will be summarized according to cumulative exposure.

Since the investigational product will be administered via an IV infusion, the summary of compliance data is not applicable for this study.

Safety analyses will be conducted using data from the Safety Population. Safety variables include TEAEs, clinical laboratory values and vital signs. No formal inferential analyses will be conducted for safety variables, unless otherwise noted.

Adverse events will be coded using the Medical Dictionary for Regulatory Activities (MedDRA). An AE will be considered treatment-emergent if it occurs after the first dose of investigational product (including the duration of infusion) and within 28 days after a subject’s last dose of investigational product.

The overall incidence of subjects having at least one AE will be summarized by dose level. The incidence of TEAEs will be summarized by dose level, system organ class (SOC), and preferred term (PT); each subject will be counted only once per SOC and PT. Similar summaries will be produced for SAEs and AEs leading to discontinuation. The maximum intensity of AEs and the maximum relationship to investigational product will also be summarized by dose level, SOC, and PT.

Clinical laboratory values will be compared to normal ranges and flagged for levels of clinical concern. Summaries will focus on the frequencies of abnormal values as well as within-subject changes observed during the study.

Descriptive summaries for all reported values and change from baseline values will be summarized by laboratory test category, dose level, and visit.

Vital signs will be examined for changes during the study that may be attributed to exposure to investigational product.

Vital signs (systolic and diastolic blood pressure, pulse rate, respiratory rate, body weight, and body temperature) will be summarized by parameter, dose level, and visit using appropriate descriptive statistics.

All efficacy measures will be summarized using descriptive statistics by dose level and visit.

All PK parameters will be summarized using descriptive statistics by dose level and visit. Descriptive statistics for serum concentrations will include n, number of subjects with concentrations below the level of quantification (BLQ), mean, SD, coefficient of variation, median, minimum, and maximum. For descriptive summaries, serum concentrations reported as BLQ will be set to zero.

All PD parameters will be summarized by dose level and visit.

The incidence of ADAs will be summarized by dose level and visit.

No interim analysis is planned for this study.

### Example 2 - Results from A Phase 1b, Multicenter, Open-Label Study to Evaluate the Safety and Tolerability, Efficacy, Pharmacokinetics, and Pharmacodynamics of Antibody A in Subjects with Adult-Onset Still’s Disease

Presented herein in Example 2 are certain results of the study conducted according to the methods as described in Example 1.

The first cohort of six patients, the 7 mg/kg cohort, completed the study described in Example 1. There were no serious adverse events attributable to Antibody A in this cohort.

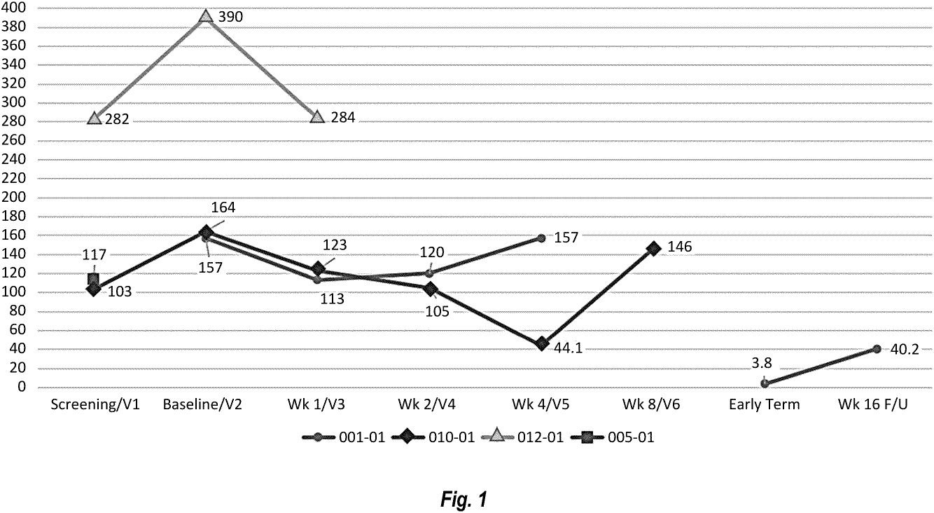

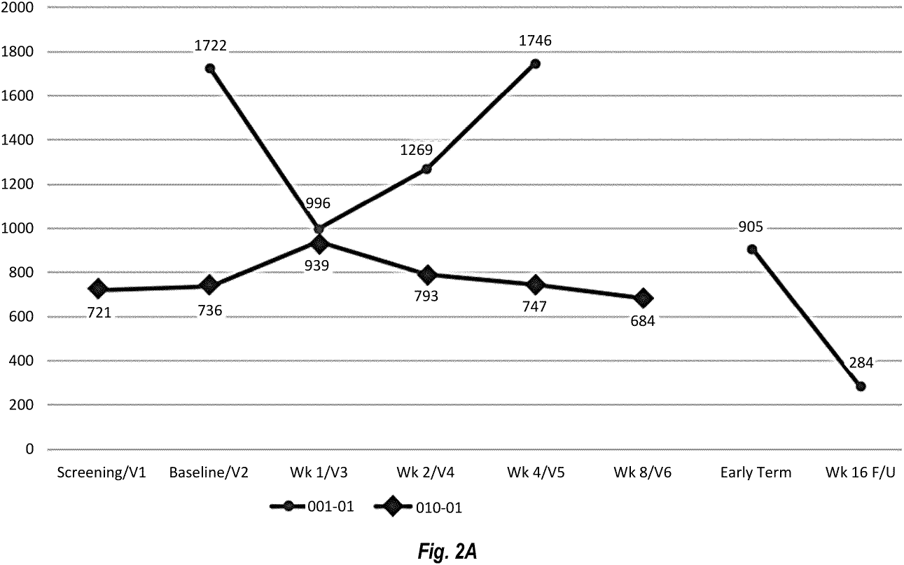

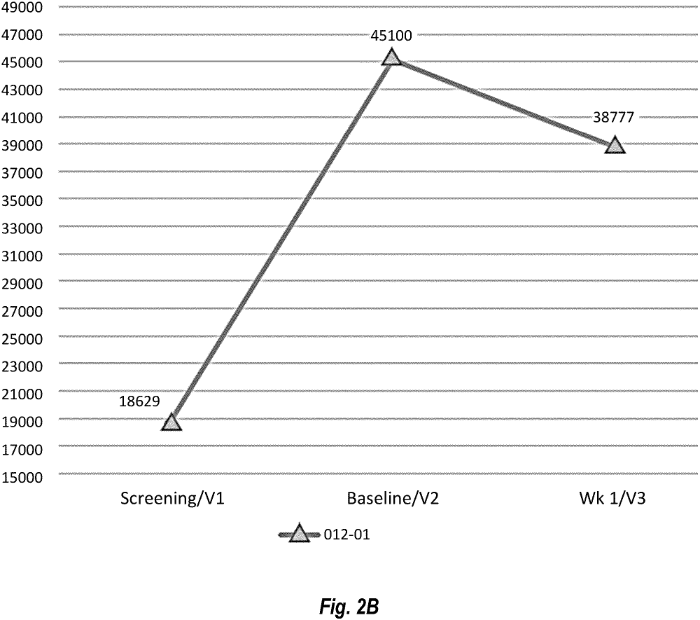

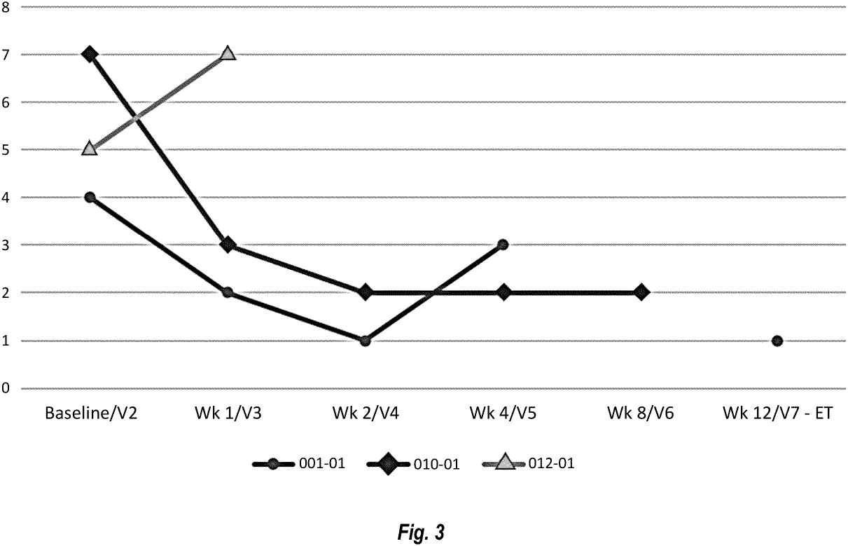

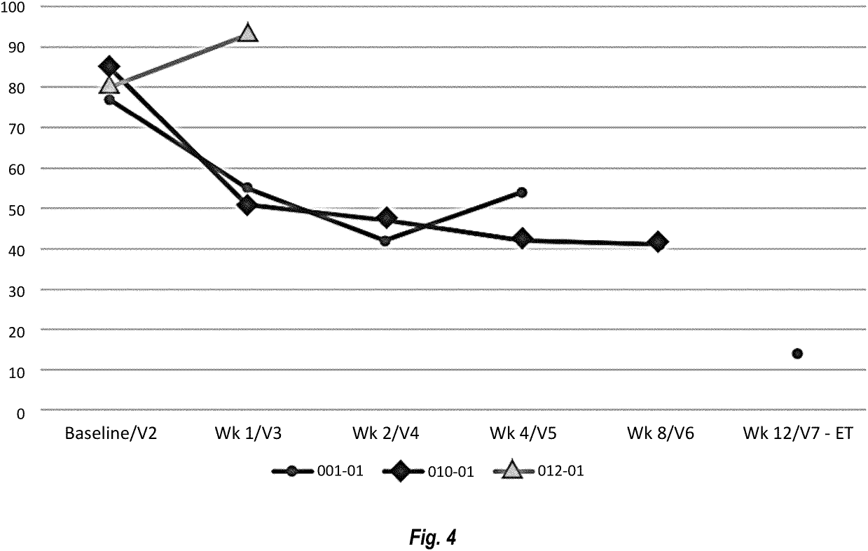

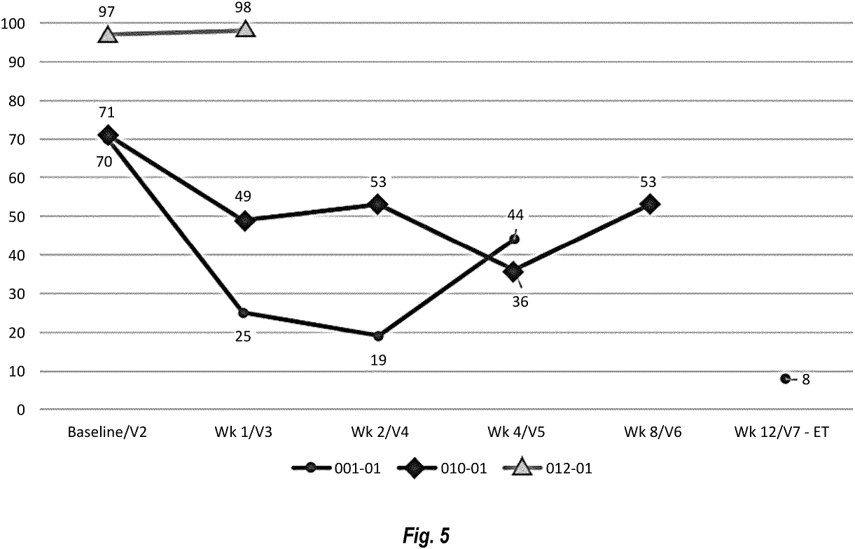

Some of the patients in the first cohort also experienced a reduction in key markers of inflammation. FIG. 1 shows CRP concentrations by visit for four patients in the first cohort. FIGS. 2A and 2B show ferritin concentrations by visit for three patients (two patients in FIG. 2A; one patient in FIG. 2B) in the first cohort. FIG. 3 shows modified Pouchot score by visit for three patients in the first cohort. FIG. 4 shows physician global assessment (PhGA) score by visit for three patients in the first cohort. FIG. 5 shows patient global assessment (PtGA) score by visit for three patients in the first cohort.

A second cohort will be treated according to Example 1: the 14 mg/kg group.

### Example 3 - A Study to Evaluate the Safety and Tolerability, Efficacy, Pharmacokinetics, and Pharmacodynamics of Antibody A in Subjects With Systemic-Onset Juvenile Idiopathic Arthritis

A study of Antibody A will be conducted in patients with systemic-onset juvenile idiopathic arthritis. The subjects will be tested according to a similar protocol as described in Example 1.

The following table provides the sequences referred to in this application.

TABLE 6

Table of Sequences

| SEQ ID NO | DESCRIPTION | SEQUENCE |
|---|---|---|
| 1 | Antibody 1 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 2 | Antibody 1 HCDR1 | Ser Gly Gly Tyr Tyr Trp Ser |
| 3 | Antibody 1 HCDR2 | Ser Ile Tyr Tyr Ser Gly Ser Thr Tyr Tyr Asn Pro Ser Leu Lys Ser |
| 4 | Antibody 1 HCDR3 | Thr Pro Ala Tyr Asp Gly Asp Ala Arg Ala Asp Phe Phe Asp Val |
| 5 | Antibody 1 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys |
| | | Gln Gln Ser Tyr Ser Thr Pro Trp Thr Phe Gly Gln Gly Thr Lys Leu Glu Ile Lys |
| 6 | Antibody 1 LCDR1 | Arg Ala Ser Gln Gly Ile Ser Ser Trp Leu Ala |
| 7 | Antibody 1 LCDR2 | Lys Ala Ser Thr Leu Glu Ser |
| 8 | Antibody 1 LCDR3 | Gln Gln Ser Tyr Ser Thr Pro Trp Thr |
| 9 | Antibody 1_GL VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ser Gly Gly Tyr Tyr Trp Ser Trp Ile Arg Gln Pro Pro Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 10 | Antibody 1_GL HCDR1 | Ser Gly Gly Tyr Tyr Trp Ser |
| 11 | Antibody 1_GL HCDR2 | Ser Ile Tyr Tyr Ser Gly Ser Thr Tyr Tyr Asn Pro Ser Leu Lys Ser |
| 12 | Antibody 1_GL HCDR3 | Thr Pro Ala Tyr Asp Gly Asp Ala Arg Ala Asp Phe Phe Asp Val |
| 13 | Antibody 1_GL VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly Ser Gly Thr Glu Phe Thr Leu Thr Ile Ser Ser Leu Gln Pro Asp Asp Phe Ala Thr Tyr Tyr Cys Gln Gln Ser Tyr Ser Thr Pro Trp Thr Phe Gly Gln Gly Thr Lys Leu Glu Ile Lys |
| 14 | Antibody 1_GL LCDR1 | Arg Ala Ser Gln Gly Ile Ser Ser Trp Leu Ala |
| 15 | Antibody 1_GL LCDR2 | Lys Ala Ser Thr Leu Glu Ser |
| 16 | Antibody 1_GL LCDR3 | Gln Gln Ser Tyr Ser Thr Pro Trp Thr |
| 17 | Antibody 2 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 18 | Antibody 2 HCDR1 | Ser Gly Gly Tyr Tyr Trp Ser |
| 19 | Antibody 2 HCDR2 | Ser Ile Tyr Tyr Ser Gly Ser Thr Tyr Tyr Asn Pro Ser Leu Lys Ser |
| 20 | Antibody 2 HCDR3 | Thr Pro Ala Tyr Asp Gly Asp Ala Arg Ala Asp Phe Phe Asp Val |
| 21 | Antibody 2 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Asp Ile Ser Phe Pro Pro Trp Thr Phe Gly Gln Gly Thr Lys Leu Glu Ile Lys |
| 22 | Antibody 2 LCDR1 | Arg Ala Ser Gln Gly Ile Ser Ser Trp Leu Ala |
| 23 | Antibody 2 LCDR2 | Lys Ala Ser Thr Leu Glu Ser |
| 24 | Antibody 2 LCDR3 | Gln Asp Ile Ser Phe Pro Pro Trp Thr |
| 25 | Antibody 3 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 26 | Antibody 3 HCDR1 | Ser Gly Gly Tyr Tyr Trp Ser |
| 27 | Antibody 3 HCDR2 | Ser Ile Tyr Tyr Ser Gly Ser Thr Tyr Tyr Asn Pro Ser Leu Lys Ser |
| 28 | Antibody 3 HCDR3 | Thr Pro Ala Tyr Asp Gly Asp Ala Arg Ala Asp Phe Phe Asp Val |
| 29 | Antibody 3 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser Leu Tyr Pro Pro Trp Thr Phe Gly Gln Gly Thr Lys Leu Glu Ile Lys |
| 30 | Antibody 3 LCDR1 | Arg Ala Ser Gln Gly Ile Ser Ser Trp Leu Ala |
| 31 | Antibody 3 LCDR2 | Lys Ala Ser Thr Leu Glu Ser |
| 32 | Antibody 3 LCDR3 | Gln Gln Ser Leu Tyr Pro Pro Trp Thr |
| 33 | Antibody 4 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 34 | Antibody 4 HCDR1 | Ser Gly Gly Tyr Tyr Trp Ser |
| 35 | Antibody 4 HCDR2 | Ser Ile Tyr Tyr Ser Gly Ser Thr Tyr Tyr Asn Pro Ser Leu Lys Ser |
| 36 | Antibody 4 HCDR3 | Thr Pro Ala Tyr Asp Gly Asp Ala Arg Ala Asp Phe Phe Asp Val |
| 37 | Antibody 4 VL | Asp Ile Val Met Thr Gln Ser Pro Ser Thr Leu Ser Ala Ser Val Gly Asp Arg Val Thr Ile Thr |
| | | Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His His Pro Asn Trp Asp Phe Gly Gln Gly Thr Lys Leu Glu Ile Lys |
| 38 | Antibody 4 LCDR1 | Arg Ala Ser Gln Gly Ile Ser Ser Trp Leu Ala |
| 39 | Antibody 4 LCDR2 | Lys Ala Ser Thr Leu Glu Ser |
| 40 | Antibody 4 LCDR3 | Gln Gln Ser His His Pro Asn Trp Asp |
| 41 | Antibody 5 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Asp Gly Asp Ala Arg Ala Asp Phe Phe Asp Val Trp Gly Arg Gly Thr Leu Val Thr Val Ser Ser |
| 42 | Antibody 5 HCDR1 | Ser Gly Gly Tyr Tyr Trp Ser |
| 43 | Antibody 5 HCDR2 | Ser Ile Tyr Tyr Ser Gly Ser Thr Tyr Tyr Asn Pro Ser Leu Lys Ser |
| 44 | Antibody 5 HCDR3 | Thr Pro Ala Tyr Asp Gly Asp Ala Arg Ala Asp Phe Phe Asp Val |
| 45 | Antibody 5 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser Leu Ile Pro Gln Trp Asp Phe Gly Gln Gly Thr Lys Leu Glu Ile Lys |
| 46 | Antibody 5 LCDR1 | Arg Ala Ser Gln Gly Ile Ser Ser Trp Leu Ala |
| 47 | Antibody 5 LCDR2 | Lys Ala Ser Thr Leu Glu Ser |
| 48 | Antibody 5 LCDR3 | Gln Gln Ser Leu Ile Pro Gln Trp Asp |
| 49 | Antibody 6 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 50 | Antibody 6 HCDR1 | Ser Gly Gly Tyr Tyr Trp Ser |
| 51 | Antibody 6 HCDR2 | Ser Ile Tyr Tyr Ser Gly Ser Thr Tyr Tyr Asn Pro Ser Leu Lys Ser |
| 52 | Antibody 6 HCDR3 | Thr Pro Ala Tyr Phe Gly Gln Asp Arg Thr Asp Phe Phe Asp Val |
| 53 | Antibody 6 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Ala Asn Ile Ala Phe Pro Pro Trp Thr Phe Gly Gln Gly Thr Lys Leu Glu Ile Lys |
| 54 | Antibody 6 LCDR1 | Arg Ala Ser Gln Gly Ile Ser Ser Trp Leu Ala |
| 55 | Antibody 6 LCDR2 | Lys Ala Ser Thr Leu Glu Ser |
| 56 | Antibody 6 LCDR3 | Ala Asn Ile Ala Phe Pro Pro Trp Thr |
| 57 | Antibody 6_GL VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ser Gly Gly Tyr Tyr Trp Ser Trp Ile Arg Gln Pro Pro Gly |
| | | Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 58 | Antibody 6_GL HCDR1 | Ser Gly Gly Tyr Tyr Trp Ser |
| 59 | Antibody 6_GL HCDR2 | Ser Ile Tyr Tyr Ser Gly Ser Thr Tyr Tyr Asn Pro Ser Leu Lys Ser |
| 60 | Antibody 6_GL HCDR3 | Thr Pro Ala Tyr Phe Gly Gln Asp Arg Thr Asp Phe Phe Asp Val |
| 61 | Antibody 6_GL VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly Ser Gly Thr Glu Phe Thr Leu Thr Ile Ser Ser Leu Gln Pro Asp Asp Phe Ala Thr Tyr Tyr Cys Ala Asn Ile Ala Phe Pro Pro Trp Thr Phe Gly Gln Gly Thr Lys Leu Glu Ile Lys |
| 62 | Antibody 6_GL LCDR1 | Arg Ala Ser Gln Gly Ile Ser Ser Trp Leu Ala |
| 63 | Antibody 6_GL LCDR2 | Lys Ala Ser Thr Leu Glu Ser |
| 64 | Antibody 6_GL | Ala Asn Ile Ala Phe Pro Pro Trp Thr |
| 65 | Antibody 7 VH | Gln Val Gln Leu Gln Gln Ser Gly Pro Arg Leu Val Lys Pro Ser Gln Thr Leu Ser Leu Thr Cys Thr Val Ser Gly Gly Ser Ile Ser Ser Gly Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 66 | Antibody 7 HCDR1 | Ser Gly Gly Tyr Tyr Trp Ser |
| 67 | Antibody 7 HCDR2 | Ser Ile Tyr Tyr Ser Gly Ser Thr Tyr Tyr Asn Pro Ser Leu Lys Ser |
| 68 | Antibody 7 HCDR3 | Thr Pro Ala Tyr Phe Gly Gln Asp Arg Thr Asp Phe Phe Asp Val |
| 69 | Antibody 7 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His His Pro Pro Trp Thr Phe Gly Gln Gly Thr Lys Leu Glu Ile Lys |
| 70 | Antibody 7 LCDR1 | Arg Ala Ser Gln Gly Ile Ser Ser Trp Leu Ala |
| 71 | Antibody 7 LCDR2 | Lys Ala Ser Thr Leu Glu Ser |
| 72 | Antibody 7 LCDR3 | Gln Gln Ser His His Pro Pro Trp Thr |
| 73 | Antibody 7_GL VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ser Gly Gly Tyr Tyr Trp Ser Trp Ile Arg Gln Pro Pro Gly Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 74 | Antibody 7_GL HCDR1 | Ser Gly Gly Tyr Tyr Trp Ser |
| 75 | Antibody 7_GL HCDR2 | Ser Ile Tyr Tyr Ser Gly Ser Thr Tyr Tyr Asn Pro Ser Leu Lys Ser |
| 76 | Antibody 7_GL HCDR3 | Thr Pro Ala Tyr Phe Gly Gln Asp Arg Thr Asp Phe Phe Asp Val |
| 77 | Antibody 7_GL VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly |
| | | Ser Gly Thr Glu Phe Thr Leu Thr Ile Ser Ser Leu Gln Pro Asp Asp Phe Ala Thr Tyr Tyr Cys Gln Gln Ser His His Pro Pro Trp Thr Phe Gly Gln Gly Thr Lys Leu Glu Ile Lys |
| 78 | Antibody 7_GL LCDR1 | Arg Ala Ser Gln Gly Ile Ser Ser Trp Leu Ala |
| 79 | Antibody 7_GL LCDR2 | Lys Ala Ser Thr Leu Glu Ser |
| 80 | Antibody 7_GL LCDR3 | Gln Gln Ser His His Pro Pro Trp Thr |
| 81 | Antibody 8_GL VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ala Gly Gly Tyr Tyr Trp Ser Trp Ile Arg Gln Pro Pro Gly Lys Gly Leu Glu Trp Ile Gly Ser Leu Tyr Tyr Ser Gly Ser Thr Tyr Tyr Asn Pro Ser Leu Lys Gly Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 82 | Antibody 8_GL HCDR1 | Ala Gly Gly Tyr Tyr Trp Ser |
| 83 | Antibody 8_GL HCDR2 | Ser Leu Tyr Tyr Ser Gly Ser Thr Tyr Tyr Asn Pro Ser Leu Lys Gly |
| 84 | Antibody 8_GL HCDR3 | Thr Pro Ala Tyr Phe Gly Gln Asp Arg Thr Asp Phe Phe Asp Val |
| 85 | Antibody 8_GL VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly Ser Gly Thr Glu Phe Thr Leu Thr Ile Ser Ser Leu Gln Pro Asp Asp Phe Ala Thr Tyr Tyr Cys Gln Gln Ser His His Pro Pro Trp Thr Phe Gly Gln Gly Thr Lys Leu Glu Ile Lys |
| 86 | Antibody 8_GL LCDR1 | Arg Ala Ser Gln Gly Ile Ser Ser Trp Leu Ala |
| 87 | Antibody 8_GL LCDR2 | Lys Ala Ser Thr Leu Glu Ser |
| 88 | Antibody 8_GL LCDR3 | Gln Gln Ser His His Pro Pro Trp Thr |
| 89 | Antibody 9 VH | Gln Val Gln Leu Gln Gln Ser Gly Pro Arg Leu Val Glu Pro Ser Gln Thr Leu Ser Leu Thr Cys Thr Val Ser Gly Gly Ser Ile Ser Ser Asp Gly Tyr Tyr Trp Ser Trp Ile Arg Gln Pro Ala Gly |
| | | Lys Gly Leu Glu Trp Ile Gly Ser Ile Tyr Tyr Ser Gly Ser Thr Tyr Tyr Asn Pro Ser Leu Lys Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 90 | Antibody 9 HCDR1 | Ser Asp Gly Tyr Tyr Trp Ser |
| 91 | Antibody 9 HCDR2 | Ser Ile Tyr Tyr Ser Gly Ser Thr Tyr Tyr Asn Pro Ser Leu Lys Ser |
| 92 | Antibody 9 HCDR3 | Thr Pro Ala Tyr Phe Gly Gln Asp Arg Thr Asp Phe Phe Asp Val |
| 93 | Antibody 9 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Arg Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His His Pro Pro Trp Thr Phe Gly Gln Gly Thr Lys Leu Glu Ile Lys |
| 94 | Antibody 9 LCDR1 | Arg Ala Ser Gln Gly Ile Ser Ser Trp Leu Ala |
| 95 | Antibody 9 LCDR2 | Lys Ala Ser Thr Leu Glu Ser |
| 96 | Antibody 9 LCDR3 | Gln Gln Ser His His Pro Pro Trp Thr |
| 97 | Antibody 10 VH | Gln Val Gln Leu Gln Gln Ser Gly Pro Arg Leu Val Glu Pro Ser Gln Thr Leu Ser Leu Thr Cys Thr Val Ser Gly Gly Ser Ile Ser Ser Asp Gly Tyr Tyr Trp Ser Trp Ile Arg Gln Pro Ala Gly Lys Gly Leu Glu Trp Ile Gly Ser Ile Tyr Tyr Ser Gly Ser Thr Tyr Tyr Asn Pro Ser Leu Arg Ser Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 98 | Antibody 10 HCDR1 | Ser Asp Gly Tyr Tyr Trp Ser |
| 99 | Antibody 10 HCDR2 | Ser Ile Tyr Tyr Ser Gly Ser Thr Tyr Tyr Asn Pro Ser Leu Arg Ser |
| 100 | Antibody 10 HCDR3 | Thr Pro Ala Tyr Phe Gly Gln Asp Arg Thr Asp Phe Phe Asp Val |
| 101 | Antibody 10 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu |
| | | Ala Trp Tyr Gln Gln Lys Pro Gly Gly Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His His Pro Pro Trp Thr Phe Ser Gln Gly Thr Lys Leu Glu Ile Lys |
| 102 | Antibody 10 LCDR1 | Arg Ala Ser Gln Gly Ile Ser Ser Trp Leu Ala |
| 103 | Antibody 10 LCDR2 | Lys Ala Ser Thr Leu Glu Ser |
| 104 | Antibody 10 LCDR3 | Gln Gln Ser His His Pro Pro Trp Thr |
| 105 | Antibody 11 VH | Gln Val Gln Leu Gln Gln Ser Gly Pro Arg Leu Val Glu Pro Ser Gln Thr Leu Ser Leu Thr Cys Thr Val Ser Gly Gly Ser Ile Ser Ala Asp Gly Tyr Tyr Trp Ser Trp Ile Arg Gln Pro Ala Gly Lys Gly Leu Glu Trp Ile Gly Ser Leu Tyr Tyr Ser Gly Ser Thr Tyr Tyr Asn Pro Ser Leu Arg Gly Arg Val Thr Ile Ser Gly Asp Thr Pro Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 106 | Antibody 11 HCDR1 | Ala Asp Gly Tyr Tyr Trp Ser |
| 107 | Antibody 11 HCDR2 | Ser Leu Tyr Tyr Ser Gly Ser Thr Tyr Tyr Asn Pro Ser Leu Arg Gly |
| 108 | Antibody 11 HCDR3 | Thr Pro Ala Tyr Phe Gly Gln Asp Arg Thr Asp Phe Phe Asp Val |
| 109 | Antibody 11 VL | Asp Ile Val Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Gly Ala Pro Lys Val Leu Ile Tyr Lys Ala Ser Thr Leu Glu Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ser His His Pro Pro Trp Thr Phe Ser Gln Gly Thr Lys Leu Glu Ile Lys |
| 110 | Antibody 11 LCDR1 | Arg Ala Ser Gln Gly Ile Ser Ser Trp Leu Ala |
| 111 | Antibody 11 LCDR2 | Lys Ala Ser Thr Leu Glu Ser |
| 112 | Antibody 11 LCDR3 | Gln Gln Ser His His Pro Pro Trp Thr |
| 113 | Antibody 11_GL VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ala Asp Gly Tyr Tyr Trp Ser Trp Ile Arg Gln Pro Pro Gly Lys Gly Leu Glu Trp Ile Gly Ser Leu Tyr Tyr Ser Gly Ser Thr Tyr Tyr Asn Pro Ser Leu Arg |
| | | Gly Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 114 | Antibody 11_GL HCDR1 | Ala Asp Gly Tyr Tyr Trp Ser |
| 115 | Antibody 11_GL HCDR2 | Ser Leu Tyr Tyr Ser Gly Ser Thr Tyr Tyr Asn Pro Ser Leu Arg Gly |
| 116 | Antibody 11_GL HCDR3 | Thr Pro Ala Tyr Phe Gly Gln Asp Arg Thr Asp Phe Phe Asp Val |
| 117 | Antibody 11_GL VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly Ser Gly Thr Glu Phe Thr Leu Thr Ile Ser Ser Leu Gln Pro Asp Asp Phe Ala Thr Tyr Tyr Cys Gln Gln Ser His His Pro Pro Trp Thr Phe Gly Gln Gly Thr Lys Leu Glu Ile Lys |
| 118 | Antibody 11_GL LCDR1 | Arg Ala Ser Gln Gly Ile Ser Ser Trp Leu Ala |
| 119 | Antibody 11_GL LCDR2 | Lys Ala Ser Thr Leu Glu Ser |
| 120 | Antibody 11_GL LCDR3 | Gln Gln Ser His His Pro Pro Trp Thr |
| 121 | Antibody 12_GL (Antibody A) VH | Gln Val Gln Leu Gln Glu Ser Gly Pro Gly Leu Val Lys Pro Ser Glu Thr Leu Ser Leu Thr Cys Thr Val Ser Gly Gly Ser Ile Ser Ala Asp Gly Tyr Tyr Trp Ser Trp Ile Arg Gln Pro Pro Gly Lys Gly Leu Glu Trp Ile Gly Ser Leu Tyr Tyr Ser Gly Ser Thr Tyr Tyr Asn Pro Ser Leu Lys Gly Arg Val Thr Ile Ser Gly Asp Thr Ser Lys Asn Gln Phe Ser Leu Lys Leu Ser Ser Val Thr Ala Ala Asp Thr Ala Val Tyr Tyr Cys Ala Arg Thr Pro Ala Tyr Phe Gly Gln Asp Arg Thr Asp Phe Phe Asp Val Trp Gly Arg Gly Thr Leu Val Thr Val Ser Ser |
| 122 | Antibody 12_GL (Antibody A) HCDR1 | Ala Asp Gly Tyr Tyr Trp Ser |
| 123 | Antibody 12_GL (Antibody A) HCDR2 | Ser Leu Tyr Tyr Ser Gly Ser Thr Tyr Tyr Asn Pro Ser Leu Lys Gly |
| 124 | Antibody 12_GL (Antibody A) HCDR3 | Thr Pro Ala Tyr Phe Gly Gln Asp Arg Thr Asp Phe Phe Asp Val |
| 125 | Antibody 12_GL (Antibody A) VL | Asp Ile Gln Met Thr Gln Ser Pro Ser Thr Leu Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Val Leu Ile Tyr Lys Ala Ser Thr Leu Glu Ser Gly Val Pro Ser Arg Phe Ser Gly Ser Gly Ser Gly Thr Glu Phe Thr Leu Thr Ile Ser Ser Leu Gln Pro Asp Asp Phe Ala Thr Tyr Tyr Cys Gln Gln Ser His His Pro Pro Trp Thr Phe Gly Gln Gly Thr Lys Leu Glu Ile Lys |
| 126 | Antibody 12_GL (Antibody A) LCDR1 | Arg Ala Ser Gln Gly Ile Ser Ser Trp Leu Ala |
| 127 | Antibody 12_GL (Antibody A) LCDR2 | Lys Ala Ser Thr Leu Glu Ser |
| 128 | Antibody 12_GL (Antibody A) LCDR3 | Gln Gln Ser His His Pro Pro Trp Thr |
| 129 | HCDR1 | Gly Tyr Tyr Phe His |
| 130 | HCDR2 | Arg Ile Asp Pro Glu Asp Asp Ser Thr Lys Tyr Ala Glu Arg Phe Lys Asp |
| 131 | HCDR3 | Trp Arg Ile Tyr Arg Asp Ser Ser Gly Arg Pro Phe Tyr Val Met Asp Ala |
| 132 | LCDR1 | Leu Ala Ser Glu Asp Ile Tyr Thr Tyr Leu Thr |
| 133 | LCDR2 | Gly Ala Asn Lys Leu Gln Asp |
| 134 | LCDR3 | Leu Gln Gly Ser Lys Phe Pro Leu Thr |
| 135 | Human IL-18 | Met Ala Ala Glu Pro Val Glu Asp Asn Cys Ile Asn Phe Val Ala Met Lys Phe Ile Asp Asn Thr Leu Tyr Phe Ile Ala Glu Asp Asp Glu Asn Leu Glu Ser Asp Tyr Phe Gly Lys Leu Glu Ser Lys Leu Ser Val Ile Arg Asn Leu Asn Asp Gln Val Leu Phe Ile Asp Gln Gly Asn Arg Pro Leu Phe Glu Asp Met Thr Asp Ser Asp Cys Arg Asp Asn Ala Pro Arg Thr Ile Phe Ile Ile Ser Met Tyr Lys Asp Ser Gln Pro Arg Gly Met Ala Val Thr Ile Ser Val Lys Cys Glu Lys Ile Ser Thr Leu Ser Cys Glu Asn Lys Ile Ile Ser Phe Lys Glu Met Asn Pro Pro Asp Asn Ile Lys Asp Thr Lys Ser Asp Ile Ile Phe Phe Gln Arg Ser Val Pro Gly His Asp Asn Lys Met Gln Phe Glu Ser Ser Ser Tyr Glu Gly Tyr Phe Leu Ala Cys Glu Lys Glu Arg Asp Leu Phe Lys Leu Ile Leu Lys Lys Glu Asp Glu Leu Gly Asp Arg Ser Ile Met Phe Thr Val Gln Asn Glu Asp |
| 136 | Human Il-18 (PN) | atggctgctg aaccagtaga agacaattgc atcaactttg tggcaatgaa atttattgac 60 |
| | | aatacgcttt actttatagc tgaagatgat gaaaacctgg aatcagatta ctttggcaag 120 cttgaatcta aattatcagt cataagaaat ttgaatgacc aagttctctt cattgaccaa 180 ggaaatcggc ctctatttga agatatgact gattctgact gtagagataa tgcaccccgg 240 accatattta ttataagtat gtataaagat agccagccta gaggtatggc tgtaactatc 300 tctgtgaagt gtgagaaaat ttcaactctc tcctgtgaga acaaaattat ttcctttaag 360 gaaatgaatc ctcctgataa catcaaggat acaaaaagtg acatcatatt ctttcagaga 420 agtgtcccag gacatgataa taagatgcaa tttgaatctt catcatacga aggatacttt 480 ctagcttgtg aaaaagagag agaccttttt aaactcattt tgaaaaaaga ggatgaattg 540 ggggatagat ctataatgtt cactgttcaa aacgaagact ag 582 |
| 137 | H1 heavy chain | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Trp Val Arg Gln Ala Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Glu Asp Thr Ser Thr Asp Thr Ala Tyr Met Glu Leu Ser Ser Leu Arg Ser Glu Asp Thr Ala Val Tyr Tyr Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser Ala Ser Thr Lys Gly Pro Ser Val Phe Pro Leu Ala Pro Ser Ser Lys Ser Thr Ser Gly Gly Thr Ala Ala Leu Gly Cys Leu Val Lys Asp Tyr Phe Pro Glu Pro Val Thr Val Ser Trp Asn Ser Gly Ala Leu Thr Ser Gly Val His Thr Phe Pro Ala Val Leu Gln Ser Ser Gly Leu Tyr Ser Leu Ser Ser Val Val Thr Val Pro Ser Ser Ser Leu Gly Thr Gln Thr Tyr Ile Cys Asn Val Asn His Lys Pro Ser Asn Thr Lys Val Asp Lys Lys Val Glu Pro Lys Ser Cys Asp Lys Thr His Thr Cys Pro Pro Cys Pro Ala Pro Glu Leu Leu Gly Gly Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asn Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro |
| | | Arg Glu Pro Gln Val Tyr Thr Leu Pro Pro Ser Arg Asp Glu Leu Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Thr Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly Lys |
| 138 | H1 heavy chain (PN) | aggtgcagc tggtgcagag cggagccgag gtgaagaagc ctggcgccag cgtcaaggtg 60 tcctgtaagg tgtccggcga gatcagcacc ggctactact tccactgggt gaggcaggcc 120 cctggcaagg gcctggagtg gatgggcaga atcgaccccg aggacgacag caccaagtac 180 gccgagcggt tcaaggacag ggtgaccatg accgaggaca ccagcaccga taccgcctac 240 atggagctgt ccagcctgag aagcgaggat accgccgtgt actactgtac cacctggcgg 300 atctacagag acagcagcgg cagacccttc tacgtgatgg atgcctgggg ccagggcaca 360 ctagtgaccg tgtccagcgc cagcaccaag ggccccagcg tgttccccct ggcccccagc 420 agcaagagca ccagcggcgg cacagccgcc ctgggctgcc tggtgaagga ctacttcccc 480 gaaccggtga ccgtgtcctg gaacagcgga gccctgacca gcggcgtgca caccttcccc 540 gccgtgctgc agagcagcgg cctgtacagc ctgagcagcg tggtgaccgt gcccagcagc 600 agcctgggca cccagaccta catctgtaac gtgaaccaca agcccagcaa caccaaggtg 660 gacaagaagg tggagcccaa gagctgtgac aagacccaca cctgcccccc ctgccctgcc 720 cccgagctgc tgggaggccc cagcgtgttc ctgttccccc ccaagcctaa ggacaccctg 780 atgatcagca gaacccccga ggtgacctgt gtggtggtgg atgtgagcca cgaggaccct 840 gaggtgaagt tcaactggta cgtggacggc gtggaggtgc acaatgccaa gaccaagccc 900 agggaggagc agtacaacag cacctaccgg gtggtgtccg tgctgaccgt gctgcaccag 960 gattggctga acggcaagga gtacaagtgt aaggtgtcca acaaggccct gcctgcccct 1020 |
| | | atcgagaaaa ccatcagcaa ggccaagggc cagcccagag agccccaggt gtacaccctg 1080 ccccctagca gagatgagct gaccaagaac caggtgtccc tgacctgcct ggtgaagggc 1140 ttctacccca gcgacatcgc cgtggagtgg gagagcaacg gccagcccga gaacaactac 1200 aagaccaccc cccctgtgct ggacagcgat ggcagcttct tcctgtacag caagctgacc 1260 gtggacaaga gcagatggca gcagggcaac gtgttcagct gctccgtgat gcacgaggcc 1320 ctgcacaatc actacaccca gaagagcctg agcctgtccc ctggcaagtg a 1371 |
| 139 | H1 variable region | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Trp Val Arg Gln Ala Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Glu Asp Thr Ser Thr Asp Thr Ala Tyr Met Glu Leu Ser Ser Leu Arg Ser Glu Asp Thr Ala Val Tyr Tyr Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser |
| 140 | H1 variable region (PN) | caggtgcagc tggtgcagag cggagccgag gtgaagaagc ctggcgccag cgtcaaggtg 60 tcctgtaagg tgtccggcga gatcagcacc ggctactact tccactgggt gaggcaggcc 120 cctggcaagg gcctggagtg gatgggcaga atcgaccccg aggacgacag caccaagtac 180 gccgagcggt tcaaggacag ggtgaccatg accgaggaca ccagcaccga taccgcctac 240 atggagctgt ccagcctgag aagcgaggat accgccgtgt actactgtac cacctggcgg 300 atctacagag acagcagcgg cagacccttc tacgtgatgg atgcctgggg ccagggcaca 360 ctagtgaccg tgtccagc 378 |
| 141 | L2 light chain | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro Lys Leu Leu Ile Tyr Gly Ala Asn Lys Leu Gln Asp Gly Val Pro Ser Arg Phe Ser Gly Ser Gly Ser Gly Thr Asp Tyr Thr Leu Thr Ile Ser Ser Leu Gln Pro Glu Asp Phe Ala Thr Tyr Tyr Cys Leu Gln Gly Ser Lys Phe Pro Leu Thr Phe Gly Gln Gly Thr Lys Leu Glu Ile Lys Arg Thr Val |
| | | Ala Ala Pro Ser Val Phe Ile Phe Pro Pro Ser Asp Glu Gln Leu Lys Ser Gly Thr Ala Ser Val Val Cys Leu Leu Asn Asn Phe Tyr Pro Arg Glu Ala Lys Val Gln Trp Lys Val Asp Asn Ala Leu Gln Ser Gly Asn Ser Gln Glu Ser Val Thr Glu Gln Asp Ser Lys Asp Ser Thr Tyr Ser Leu Ser Ser Thr Leu Thr Leu Ser Lys Ala Asp Tyr Glu Lys His Lys Val Tyr Ala Cys Glu Val Thr His Gln Gly Leu Ser Ser Pro Val Thr Lys Ser Phe Asn Arg Gly Glu Cys |
| 142 | L2 light chain (PN) | gatatccaga tgacccagtc ccccagcagc gtgtccgcct ctgtgggcga tagagtgacc 60 atcacctgcc tggccagcga ggacatctac acctacctga cctggtatca gcagaagcct 120 ggcaaggccc ctaagctgct gatctacggc gccaacaagc tgcaggacgg cgtgcccagc 180 agattcagcg gcagcggctc cggcaccgac tacaccctga ccatcagcag cctgcagcct 240 gaggatttcg ccacctacta ctgcctgcag ggcagcaagt tccccctgac cttcggccag 300 ggcaccaagc tggagatcaa gcgtacggtg gccgccccca gcgtgttcat cttccccccc 360 agcgatgagc agctgaagag cggcaccgcc agcgtggtgt gtctgctgaa caacttctac 420 ccccgggagg ccaaggtgca gtggaaggtg gacaatgccc tgcagagcgg caacagccag 480 gagagcgtga ccgagcagga cagcaaggac tccacctaca gcctgagcag caccctgacc 540 ctgagcaagg ccgactacga gaagcacaag gtgtacgcct gtgaggtgac ccaccagggc 600 ctgtccagcc ccgtgaccaa gagcttcaac cggggcgagt gc 642 |
| 143 | L2 variable region | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro Lys Leu Leu Ile Tyr Gly Ala Asn Lys Leu Gln Asp Gly Val Pro Ser Arg Phe Ser Gly Ser Gly Ser Gly Thr Asp Tyr Thr Leu Thr Ile Ser Ser Leu Gln Pro Glu Asp Phe Ala Thr Tyr Tyr Cys Leu Gln Gly Ser Lys Phe Pro Leu Thr Phe Gly Gln Gly Thr Lys Leu Glu Ile Lys |
| 144 | L2 variable region (PN) | gatatccaga tgacccagtc ccccagcagc gtgtccgcct ctgtgggcga tagagtgacc 60 atcacctgcc tggccagcga ggacatctac acctacctga cctggtatca gcagaagcct 120 |
| | | ggcaaggccc ctaagctgct gatctacggc gccaacaagc tgcaggacgg cgtgcccagc 180 agattcagcg gcagcggctc cggcaccgac tacaccctga ccatcagcag cctgcagcct 240 gaggatttcg ccacctacta ctgcctgcag ggcagcaagt tccccctgac cttcggccag 300 ggcaccaagc tggagatcaa g 321 |
| 145 | H2 heavy chain | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Trp Val Arg Arg Arg Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Glu Asp Thr Ser Thr Asp Thr Ala Tyr Met Glu Leu Ser Ser Leu Arg Ser Glu Asp Thr Ala Val Tyr Tyr Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser Ala Ser Thr Lys Gly Pro Ser Val Phe Pro Leu Ala Pro Ser Ser Lys Ser Thr Ser Gly Gly Thr Ala Ala Leu Gly Cys Leu Val Lys Asp Tyr Phe Pro Glu Pro Val Thr Val Ser Trp Asn Ser Gly Ala Leu Thr Ser Gly Val His Thr Phe Pro Ala Val Leu Gln Ser Ser Gly Leu Tyr Ser Leu Ser Ser Val Val Thr Val Pro Ser Ser Ser Leu Gly Thr Gln Thr Tyr Ile Cys Asn Val Asn His Lys Pro Ser Asn Thr Lys Val Asp Lys Lys Val Glu Pro Lys Ser Cys Asp Lys Thr His Thr Cys Pro Pro Cys Pro Ala Pro Glu Leu Leu Gly Gly Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asn Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Asp Glu Leu Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Thr Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly Lys |
| 146 | H2 heavy chain (PN) | caggtccagc tggtacagtc tggggctgag gtgaagaagc ctggggcctc agtgaaggtc 60 tcctgcaagg tttccggaga aataagtact ggatactatt tccactgggt gcgacgaagg 120 cctggaaaag ggcttgagtg gatgggaagg attgatcctg aggatgatag tactaaatat 180 gctgagaggt tcaaagacag agtcaccatg accgaggaca catctacaga cacagcctac 240 atggagctga gcagcctgag atctgaggac acggccgtgt attactgtac cacatggcgg 300 atataccgag atagttctgg ccgccccttc tatgttatgg atgcctgggg ccaagggaca 360 ctagtcacag tctcctcagc ctccaccaag ggcccatcgg tcttccccct ggcaccctcc 420 tccaagagca cctctggggg cacagcggcc ctgggctgcc tggtcaagga ctacttcccc 480 gaaccggtga cggtgtcgtg gaactcaggc gccctgacca gcggcgtgca caccttcccg 540 gctgtcctac agtcctcagg actctactcc ctcagcagcg tggtgaccgt gccctccagc 600 agcttgggca cccagaccta catctgcaac gtgaatcaca agcccagcaa caccaaggtg 660 gacaagaaag ttgagcccaa atcttgtgac aaaactcaca catgcccacc gtgcccagca 720 cctgaactcc tggggggacc gtcagtcttc ctcttccccc caaaacccaa ggacaccctc 780 atgatctccc ggacccctga ggtcacatgc gtggtggtgg acgtgagcca cgaagaccct 840 gaggtcaagt tcaactggta cgtggacggc gtggaggtgc ataatgccaa gacaaagccg 900 cgggaggagc agtacaacag cacgtaccgt gtggtcagcg tcctcaccgt cctgcaccag 960 gactggctga atggcaagga gtacaagtgc aaggtctcca acaaagccct cccagccccc 1020 atcgagaaaa ccatctccaa agccaaaggg cagccccgag aaccacaggt gtacaccctg 1080 cccccatccc gggatgagct gaccaagaac caggtcagcc tgacctgcct ggtcaaaggc 1140 ttctatccca gcgacatcgc cgtggagtgg gagagcaatg ggcagccgga gaacaactac 1200 aagaccacgc ctcccgtgct ggactccgac ggctccttct tcctctacag caagctcacc 1260 gtggacaaga gcaggtggca gcaggggaac gtcttctcat gctccgtgat gcatgaggct 1320 ctgcacaacc actacacgca gaagagcctc tccctgtctc cgggtaaa 1368 |
| 147 | H2 variable region | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Trp Val Arg Arg Arg Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Glu Asp Thr Ser Thr Asp Thr Ala Tyr Met Glu Leu Ser Ser Leu Arg Ser Glu Asp Thr Ala Val Tyr Tyr Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser |
| 148 | H2 variable region (PN) | caggtccagc tggtacagtc tggggctgag gtgaagaagc ctggggcctc agtgaaggtc 60 tcctgcaagg tttccggaga aataagtact ggatactatt tccactgggt gcgacgaagg 120 cctggaaaag ggcttgagtg gatgggaagg attgatcctg aggatgatag tactaaatat 180 gctgagaggt tcaaagacag agtcaccatg accgaggaca catctacaga cacagcctac 240 atggagctga gcagcctgag atctgaggac acggccgtgt attactgtac cacatggcgg 300 atataccgag atagttctgg ccgccccttc tatgttatgg atgcctgggg ccaagggaca 360 ctagtcacag tctcctca 378 |
| 149 | H3 heavy chain | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Phe Val Arg Arg Arg Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Ala Asp Thr Ser Thr Asp Thr Ala Tyr Met Glu Leu Ser Ser Leu Arg Ser Glu Asp Thr Ala Thr Tyr Phe Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser Ala Ser Thr Lys Gly Pro Ser Val Phe Pro Leu Ala Pro Ser Ser Lys Ser Thr Ser Gly Gly Thr Ala Ala Leu Gly Cys Leu Val Lys Asp Tyr Phe Pro Glu Pro Val Thr Val Ser Trp Asn Ser Gly Ala Leu Thr Ser Gly Val His Thr Phe Pro Ala Val Leu Gln Ser Ser Gly Leu Tyr Ser Leu Ser Ser Val Val Thr Val Pro Ser Ser Ser Leu Gly Thr Gln Thr Tyr Ile Cys Asn Val Asn His Lys Pro Ser Asn Thr Lys Val Asp Lys Lys Val Glu Pro Lys Ser Cys Asp Lys Thr His Thr Cys Pro Pro Cys Pro Ala Pro Glu Leu Leu Gly Gly Pro Ser Val Phe Leu Phe Pro Pro Lys Pro Lys Asp Thr Leu Met Ile Ser Arg |
| | | Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asn Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Asp Glu Leu Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Thr Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly Lys |
| 150 | H3 heavy chain (PN) | caggtccagc tggtacagtc tggggctgag gtgaagaagc ctggggcctc agtgaaggtc 60 tcctgcaagg tttccggaga aataagtact ggatactatt tccactttgt gcgacgaagg 120 cctggaaaag ggcttgagtg gatgggaagg attgatcctg aggatgatag tactaaatat 180 gctgagaggt tcaaagacag agtcaccatg accgcagaca catctacaga cacagcctac 240 atggagctga gcagcctgag atctgaggac acggccactt atttttgtac cacatggcgg 300 atataccgag atagttctgg ccgccccttc tatgttatgg atgcctgggg ccaagggaca 360 ctagtcacag tctcctcagc ctccaccaag ggcccatcgg tcttccccct ggcaccctcc 420 tccaagagca cctctggggg cacagcggcc ctgggctgcc tggtcaagga ctacttcccc 480 gaaccggtga cggtgtcgtg gaactcaggc gccctgacca gcggcgtgca caccttcccg 540 gctgtcctac agtcctcagg actctactcc ctcagcagcg tggtgaccgt gccctccagc 600 agcttgggca cccagaccta catctgcaac gtgaatcaca agcccagcaa caccaaggtg 660 gacaagaaag ttgagcccaa atcttgtgac aaaactcaca catgcccacc gtgcccagca 720 cctgaactcc tggggggacc gtcagtcttc ctcttccccc caaaacccaa ggacaccctc 780 atgatctccc ggacccctga ggtcacatgc gtggtggtgg acgtgagcca cgaagaccct 840 |
| | | gaggtcaagt tcaactggta cgtggacggc gtggaggtgc ataatgccaa gacaaagccg 900 cgggaggagc agtacaacag cacgtaccgt gtggtcagcg tcctcaccgt cctgcaccag 960 gactggctga atggcaagga gtacaagtgc aaggtctcca acaaagccct cccagccccc 1020 atcgagaaaa ccatctccaa agccaaaggg cagccccgag aaccacaggt gtacaccctg 1080 cccccatccc gggatgagct gaccaagaac caggtcagcc tgacctgcct ggtcaaaggc 1140 ttctatccca gcgacatcgc cgtggagtgg gagagcaatg ggcagccgga gaacaactac 1200 aagaccacgc ctcccgtgct ggactccgac ggctccttct tcctctacag caagctcacc 1260 gtggacaaga gcaggtggca gcaggggaac gtcttctcat gctccgtgat gcatgaggct 1320 ctgcacaacc actacacgca gaagagcctc tccctgtctc cgggtaaa 1368 |
| 151 | H3 variable region | Gln Val Gln Leu Val Gln Ser Gly Ala Glu Val Lys Lys Pro Gly Ala Ser Val Lys Val Ser Cys Lys Val Ser Gly Glu Ile Ser Thr Gly Tyr Tyr Phe His Phe Val Arg Arg Arg Pro Gly Lys Gly Leu Glu Trp Met Gly Arg Ile Asp Pro Glu Asp Asp Ser Thr Lys Tyr Ala Glu Arg Phe Lys Asp Arg Val Thr Met Thr Ala Asp Thr Ser Thr Asp Thr Ala Tyr Met Glu Leu Ser Ser Leu Arg Ser Glu Asp Thr Ala Thr Tyr Phe Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser |
| 152 | H3 variable region (PN) | caggtccagc tggtacagtc tggggctgag gtgaagaagc ctggggcctc agtgaaggtc 60 tcctgcaagg tttccggaga aataagtact ggatactatt tccactttgt gcgacgaagg 120 cctggaaaag ggcttgagtg gatgggaagg attgatcctg aggatgatag tactaaatat 180 gctgagaggt tcaaagacag agtcaccatg accgcagaca catctacaga cacagcctac 240 atggagctga gcagcctgag atctgaggac acggccactt atttttgtac cacatggcgg 300 atataccgag atagttctgg ccgccccttc tatgttatgg atgcctgggg ccaagggaca 360 ctagtcacag tctcctca 378 |
| 153 | L1 light chain | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro |
| | | Lys Leu Leu Ile Tyr Gly Ala Asn Lys Leu Gln Asp Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Leu Gln Gly Ser Lys Phe Pro Leu Thr Phe Gly Gln Gly Thr Lys Leu Glu Ile Lys Arg Thr Val Ala Ala Pro Ser Val Phe Ile Phe Pro Pro Ser Asp Glu Gln Leu Lys Ser Gly Thr Ala Ser Val Val Cys Leu Leu Asn Asn Phe Tyr Pro Arg Glu Ala Lys Val Gln Trp Lys Val Asp Asn Ala Leu Gln Ser Gly Asn Ser Gln Glu Ser Val Thr Glu Gln Asp Ser Lys Asp Ser Thr Tyr Ser Leu Ser Ser Thr Leu Thr Leu Ser Lys Ala Asp Tyr Glu Lys His Lys Val Tyr Ala Cys Glu Val Thr His Gln Gly Leu Ser Ser Pro Val Thr Lys Ser Phe Asn Arg Gly Glu Cys |
| 154 | L1 light chain (PN) | gacatccaga tgacccagtc tccatcttct gtgtctgcat ctgtaggaga cagagtcacc 60 atcacttgtc tggcaagtga ggacatatac acttatttaa catggtatca gcagaaacca 120 gggaaagccc ctaagctcct gatctatggt gcaaataagt tgcaagatgg ggtcccatca 180 aggttcagcg gcagtggatc tgggacagat ttcactctca ctatcagcag cctgcagcct 240 gaagattttg caacttacta ttgtctacag ggttccaagt ttccgctcac gtttggccag 300 gggaccaagc tggagatcaa acgtacggtg gctgcaccat ctgtcttcat cttcccgcca 360 tctgatgagc agttgaaatc tggaactgcc tctgttgtgt gcctgctgaa taacttctat 420 cccagagagg ccaaagtaca gtggaaggtg gacaacgccc tccaatcggg taactcccag 480 gagagtgtca cagagcagga cagcaaggac agcacctaca gcctcagcag caccctgacg 540 ctgagcaaag cagactacga gaaacacaaa gtctacgcct gcgaagtcac ccatcagggc 600 ctgagctcgc ccgtcacaaa gagcttcaac aggggagagt gt 642 |
| 155 | L1 variable region | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro Lys Leu Leu Ile Tyr Gly Ala Asn Lys Leu Gln Asp Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Leu Gln Gly Ser Lys Phe Pro Leu Thr Phe Gly Gln Gly Thr Lys Leu Glu Ile Lys |
| 156 | L1 variable region (PN) | gacatccaga tgacccagtc tccatcttct gtgtctgcat ctgtaggaga cagagtcacc 60 atcacttgtc tggcaagtga ggacatatac acttatttaa catggtatca gcagaaacca 120 gggaaagccc ctaagctcct gatctatggt gcaaataagt tgcaagatgg ggtcccatca 180 aggttcagcg gcagtggatc tgggacagat ttcactctca ctatcagcag cctgcagcct 240 gaagattttg caacttacta ttgtctacag ggttccaagt ttccgctcac gtttggccag 300 gggaccaagc tggagatcaa a 321 |
| 157 | L3 light chain | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro Gln Leu Leu Ile Tyr Gly Ala Asn Lys Leu Gln Asp Gly Val Pro Ser Arg Phe Ser Gly Ser Gly Ser Gly Thr Asp Tyr Thr Leu Thr Ile Ser Ser Leu Gln Pro Glu Asp Glu Gly Asp Tyr Tyr Cys Leu Gln Gly Ser Lys Phe Pro Leu Thr Phe Gly Gln Gly Thr Lys Leu Glu Ile Lys Arg Thr Val Ala Ala Pro Ser Val Phe Ile Phe Pro Pro Ser Asp Glu Gln Leu Lys Ser Gly Thr Ala Ser Val Val Cys Leu Leu Asn Asn Phe Tyr Pro Arg Glu Ala Lys Val Gln Trp Lys Val Asp Asn Ala Leu Gln Ser Gly Asn Ser Gln Glu Ser Val Thr Glu Gln Asp Ser Lys Asp Ser Thr Tyr Ser Leu Ser Ser Thr Leu Thr Leu Ser Lys Ala Asp Tyr Glu Lys His Lys Val Tyr Ala Cys Glu Val Thr His Gln Gly Leu Ser Ser Pro Val Thr Lys Ser Phe Asn Arg Gly Glu Cys |
| 158 | L3 light chain (PN) | gacatccaga tgacccagtc tccatcttct gtgtctgcat ctgtaggaga cagagtcacc 60 atcacttgtc tggcaagtga ggacatatac acttatttaa catggtatca gcagaaacca 120 gggaaagccc ctcaactcct gatctatggt gcaaataagt tgcaagatgg ggtcccatca 180 aggttcagcg gcagtggatc tgggacagat tatactctca ctatcagcag cctgcagcct 240 gaagatgaag gggattacta ttgtctacag ggttccaagt ttccgctcac gtttggccag 300 gggaccaagc tggagatcaa acgtacggtg gctgcaccat ctgtcttcat cttcccgcca 360 tctgatgagc agttgaaatc tggaactgcc tctgttgtgt gcctgctgaa taacttctat 420 cccagagagg ccaaagtaca gtggaaggtg gacaacgccc tccaatcggg taactcccag 480 |
| | | gagagtgtca cagagcagga cagcaaggac agcacctaca gcctcagcag caccctgacg 540 ctgagcaaag cagactacga gaaacacaaa gtctacgcct gcgaagtcac ccatcagggc 600 ctgagctcgc ccgtcacaaa gagcttcaac aggggagagt gttag 645 |
| 159 | L3 variable region | Asp Ile Gln Met Thr Gln Ser Pro Ser Ser Val Ser Ala Ser Val Gly Asp Arg Val Thr Ile Thr Cys Leu Ala Ser Glu Asp Ile Tyr Thr Tyr Leu Thr Trp Tyr Gln Gln Lys Pro Gly Lys Ala Pro Gln Leu Leu Ile Tyr Gly Ala Asn Lys Leu Gln Asp Gly Val Pro Ser Arg Phe Ser Gly Ser Gly Ser Gly Thr Asp Tyr Thr Leu Thr Ile Ser Ser Leu Gln Pro Glu Asp Glu Gly Asp Tyr Tyr Cys Leu Gln Gly Ser Lys Phe Pro Leu Thr Phe Gly Gln Gly Thr Lys Leu Glu Ile Lys |
| 160 | L3 variable region (PN) | gacatccaga tgacccagtc tccatcttct gtgtctgcat ctgtaggaga cagagtcacc 60 atcacttgtc tggcaagtga ggacatatac acttatttaa catggtatca gcagaaacca 120 gggaaagccc ctcaactcct gatctatggt gcaaataagt tgcaagatgg ggtcccatca 180 aggttcagcg gcagtggatc tgggacagat tatactctca ctatcagcag cctgcagcct 240 gaagatgaag gggattacta ttgtctacag ggttccaagt ttccgctcac gtttggccag 300 gggaccaagc tggagatcaa a 321 |
| 161 | 2c10 rat-human IgG1 chimera | Glu Val Gln Leu Gln Gln Ser Gly Ala Glu Leu Val Arg Pro Gly Thr Ser Val Lys Leu Ser Cys Lys Val Ser Gly Glu Ile Ser Thr Gly Tyr Tyr Phe His Phe Val Arg Arg Arg Pro Gly Gln Gly Leu Glu Trp Ile Gly Arg Ile Asp Pro Glu Asp Asp Ser Thr Lys Tyr Ala Glu Arg Phe Lys Asp Arg Ala Thr Leu Thr Ala Gln Thr Ser Ser Asn Thr Ala Tyr Leu Asn Leu Ser Ser Leu Thr Ser Glu Asp Thr Ala Thr Tyr Phe Cys Thr Thr Trp Arg Ile Tyr Arg Asp Ser Ser Gly Arg Pro Phe Tyr Val Met Asp Ala Trp Gly Gln Gly Thr Leu Val Thr Val Ser Ser Ala Ser Thr Lys Gly Pro Ser Val Phe Pro Leu Ala Pro Ser Ser Lys Ser Thr Ser Gly Gly Thr Ala Ala Leu Gly Cys Leu Val Lys Asp Tyr Phe Pro Glu Pro Val Thr Val Ser Trp Asn Ser Gly Ala Leu Thr Ser Gly Val His Thr Phe Pro Ala Val Leu Gln Ser Ser Gly Leu Tyr Ser Leu Ser Ser Val Val Thr Val Pro Ser Ser Ser Leu Gly Thr Gln Thr Tyr Ile Cys Asn Val Asn His Lys Pro Ser Asn Thr Lys Val Asp Lys Lys Val Glu Pro Lys Ser Cys Asp Lys |
| | | Thr His Thr Cys Pro Pro Cys Pro Ala Pro Glu Leu Leu Gly Gly Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asn Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Asp Glu Leu Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Thr Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly Lys |
| 162 | 2c10 rat-human IgG1 chimera (PN) | gaggtccagc tacagcagtc tggggctgag cttgtgagac ctgggacctc tgtgaagtta 60 tcttgcaaag tttctggcga aataagtaca ggatactatt tccactttgt gaggcgaagg 120 cctggacagg gtctggaatg gataggaagg attgatcctg aggatgatag tactaaatat 180 gctgagaggt tcaaagacag ggcgacgctc actgcacaaa catcctccaa cacagcctac 240 ctgaacctca gcagcctgac ctctgaggac actgcaactt atttttgtac cacatggcgg 300 atataccgag atagttctgg ccgccccttc tatgttatgg atgcctgggg tcaaggaaca 360 ctagtcacag tctcctcagc ctccaccaag ggcccatcgg tcttccccct ggcaccctcc 420 tccaagagca cctctggggg cacagcggcc ctgggctgcc tggtcaagga ctacttcccc 480 gaaccggtga cggtgtcgtg gaactcaggc gccctgacca gcggcgtgca caccttcccg 540 gctgtcctac agtcctcagg actctactcc ctcagcagcg tggtgaccgt gccctccagc 600 agcttgggca cccagaccta catctgcaac gtgaatcaca agcccagcaa caccaaggtg 660 gacaagaaag ttgagcccaa atcttgtgac aaaactcaca catgcccacc gtgcccagca 720 cctgaactcc tggggggacc gtcagtcttc ctcttccccc caaaacccaa ggacaccctc 780 atgatctccc ggacccctga ggtcacatgc gtggtggtgg acgtgagcca cgaagaccct 840 |
| | | gaggtcaagt tcaactggta cgtggacggc gtggaggtgc ataatgccaa gacaaagccg 900 cgggaggagc agtacaacag cacgtaccgt gtggtcagcg tcctcaccgt cctgcaccag 960 gactggctga atggcaagga gtacaagtgc aaggtctcca acaaagccct cccagccccc 1020 atcgagaaaa ccatctccaa agccaaaggg cagccccgag aaccacaggt gtacaccctg 1080 cccccatccc gggatgagct gaccaagaac caggtcagcc tgacctgcct ggtcaaaggc 1140 ttctatccca gcgacatcgc cgtggagtgg gagagcaatg ggcagccgga gaacaactac 1200 aagaccacgc ctcccgtgct ggactccgac ggctccttct tcctctacag caagctcacc 1260 gtggacaaga gcaggtggca gcaggggaac gtcttctcat gctccgtgat gcatgaggct 1320 ctgcacaacc actacacgca gaagagcctc tccctgtctc cgggtaaa 1368 |
| 163 | 2c10 rat-human CKappa chimera | Asp Ile Gln Met Thr Gln Ser Pro Ala Ser Leu Ser Ala Ser Leu Gly Glu Thr Val Ser Ile Glu Cys Leu Ala Ser Glu Asp Ile Tyr Thr Tyr Leu Thr Trp Tyr Gln Gln Lys Pro Gly Lys Ser Pro Gln Leu Leu Ile Tyr Gly Ala Asn Lys Leu Gln Asp Gly Val Pro Ser Arg Phe Ser Gly Ser Gly Ser Gly Thr Gln Tyr Ser Leu Lys Ile Ser Gly Ile Gln Pro Glu Asp Glu Gly Asp Tyr Phe Cys Leu Gln Gly Ser Lys Phe Pro Leu Thr Phe Gly Ser Gly Thr Lys Leu Glu Ile Lys Arg Thr Val Ala Ala Pro Ser Val Phe Ile Phe Pro Pro Ser Asp Glu Gln Leu Lys Ser Gly Thr Ala Ser Val Val Cys Leu Leu Asn Asn Phe Tyr Pro Arg Glu Ala Lys Val Gln Trp Lys Val Asp Asn Ala Leu Gln Ser Gly Asn Ser Gln Glu Ser Val Thr Glu Gln Asp Ser Lys Asp Ser Thr Tyr Ser Leu Ser Ser Thr Leu Thr Leu Ser Lys Ala Asp Tyr Glu Lys His Lys Val Tyr Ala Cys Glu Val Thr His Gln Gly Leu Ser Ser Pro Val Thr Lys Ser Phe Asn Arg Gly Glu Cys |
| 164 | 2c10 rat-human CKappa chimera (PN) | gacattcaaa tgacccagtc tccagcttcc ctgtctgcat ctctgggaga aactgtctcc 60 atcgaatgtc tggcaagtga ggacatatac acttatttaa catggtatca gcagaaacca 120 gggaaatctc ctcaactcct gatctatggt gcaaataagt tgcaagatgg ggtcccatca 180 cggttcagtg gcagtggatc tggcacacag tattctctca agatcagcgg catacaacct 240 gaagatgaag gggattattt ctgtctacag ggttccaagt ttccgctcac gttcggttct 300 |
| | | gggaccaagc tggagatcaa acgtacggtg gctgcaccat ctgtcttcat cttcccgcca 360 tctgatgagc agttgaaatc tggaactgcc tctgttgtgt gcctgctgaa taacttctat 420 cccagagagg ccaaagtaca gtggaaggtg gacaacgccc tccaatcggg taactcccag 480 gagagtgtca cagagcagga cagcaaggac agcacctaca gcctcagcag caccctgacg 540 ctgagcaaag cagactacga gaaacacaaa gtctacgcct gcgaagtcac ccatcagggc 600 ctgagctcgc ccgtcacaaa gagcttcaac aggggagagt gt 642 |
| 165 | Heavy chain acceptor framework | Gln Val Gln Leu Val Gln Ser Gly Ala Glu Val Lys Lys Pro Gly Ala Ser Val Lys Val Ser Cys Lys Val Ser Gly Tyr Thr Leu Thr Glu Leu Ser Met His Trp Val Arg Gln Ala Pro Gly Lys Gly Leu Glu Trp Met Gly Gly Phe Asp Pro Glu Asp Gly Glu Thr Ile Tyr Ala Gln Lys Phe Gln Gly Arg Val Thr Met Thr Glu Asp Thr Ser Thr Asp Thr Ala Tyr Met Glu Leu Ser Ser Leu Arg Ser Glu Asp Thr Ala Val Tyr Tyr Cys Ala Thr |
| 166 | Light chain acceptor framework | Asp Ile Gln Met Thr Gln Ser Pro Ser Ser Val Ser Ala Ser Val Gly Asp Arg Val Thr Ile Thr Cys Arg Ala Ser Gln Gly Ile Ser Ser Trp Leu Ala Trp Tyr Gln Gln Lys Pro Gly Lys Ala Pro Lys Leu Leu Ile Tyr Ala Ala Ser Ser Leu Gln Ser Gly Val Pro Ser Arg Phe Ser Gly Ser Gly Ser Gly Thr Asp Phe Thr Leu Thr Ile Ser Ser Leu Gln Pro Glu Asp Phe Ala Thr Tyr Tyr Cys Gln Gln Ala Asn Ser Phe Pro |
| 167 | JH6 amino acid sequence added to SEQ ID NO: 37 | Trp Gly Gln Gly Thr Leu Val Thr Val Ser Ser |
| 168 | Jkappa 2 amino acid sequence added to SEQ ID NO: 38 | Phe Gly Gln Gly Thr Lys Leu Glu Ile Lys |

What is claimed is:

1. A method of treating adult-onset Still’s disease (AOSD) or systemic-onset juvenile idiopathic arthritis (SoJIA), comprising administering to a human subject diagnosed with AOSD or SoJIA an effective amount of an anti-IL-18 antibody wherein the anti-IL-18 antibody comprises the following six CDRs:
(a) a heavy chain CDR having an amino acid sequence of SEQ ID NO: 122;
(b) a heavy chain CDR having an amino acid sequence of SEQ ID NO: 123;
(c) a heavy chain CDR having an amino acid sequence of SEQ ID NO: 124;
(d) a light chain CDR having an amino acid sequence of SEQ ID NO: 126;
(e) a light chain CDR having an amino acid sequence of SEQ ID NO: 127; and
(f) a light chain CDR having an amino acid sequence of SEQ ID NO: 128;
and wherein the anti-IL-18 antibody is administered at a dose of 4 mg/kg, 7 mg/kg, or 14 mg/kg.

2. The method of claim 1, wherein the subject is diagnosed with AOSD based on having 5 or more of the following criteria, 2 of which are major:
(a) Major Criteria
i. Fever >39° C., lasting 1 week or longer
ii. Arthralgia or arthritis, lasting 2 weeks or longer
iii. Typical rash
iv. Leukocytes >10,000 mm³ with >80% polymorphonuclear cells
(b) Minor Criteria
v. Sore throat
vi. Recent development of significant lymphadenopathy
vii. Hepatomegaly or splenomegaly
viii. Abnormal liver function tests
ix. Negative tests for antinuclear antibody (IF) and rheumatoid factor (IgM).

3. The method of claim 1, wherein the subject has reported a recurring fever >38° C. within the last 5 days of screening and baseline visits.

4. The method of claim 1, wherein if the subject is undergoing:
treatment with NSAIDs, the subject is on a stable dose for at least 48 hours prior to a baseline visit,
treatment with glucocorticoids, the subject is on a stable dose for at least 48 hours prior to a baseline visit, and/or
treatment with conventional DMARDs, the subject is on a stable dose for at least 12 weeks prior to a baseline visit.

5. (canceled)

6. (canceled)

7. The method of claim 1, wherein if the subject has received treatment with biological DMARDs, the subject has undergone the required washout period prior to a baseline visit, wherein the required washout period is as follows:
a) anakinra - 1 week;
b) etanercept, rilonacept - 4 weeks;
c) adalimumab, certolizumab, infliximab, golimumab, abatacept, tocilizumab and canakinumab - 8 weeks; and
d) rituximab - 36 weeks.

8. The method of claim 1, wherein the subject does not have a serum creatinine concentration of > 1.5 mg/dl, and/or hemoglobin ≤ 10 g/dl, neutrophils ≤ 1,500/µl and/or thrombocytes ≤75,000/µl.

9. (canceled)

10. (canceled)

11. The method of claim 1, wherein the subject has elevated free or total serum IL-18 levels.

12. The method of claim 1, wherein the level of serum IL-18 is measured prior to administration of the anti-IL-18 antibody; or after administration of the anti-IL18 antibody, optionally as a marker for effectiveness of treatment.

13. (canceled)

14. The method of claim 1, wherein the level of serum IL-18 is elevated as compared to levels in a subject without AOSD or SoJIA or as compared to a negative control.

15. (canceled)

16. The method of claim 1, wherein the level of serum IL-18 is free IL-18, optionally wherein the level of free IL-18 is calculated, or wherein the level of serum IL-18 is total IL-18.

17. (canceled)

18. (canceled)

19. (canceled)

20. The method of claim 1, wherein the anti-IL-18 antibody comprises a VH domain having an amino acid sequence that is at least 90% identical to the full sequence of SEQ ID NO: 121, and/or wherein the anti-IL-18 antibody comprises a VH domain having an amino acid sequence that is identical to the full sequence of SEQ ID NO: 121, and/or wherein the anti-IL-18 antibody comprises a VL domain having an amino acid sequence that is at least 90% identical to the full sequence of SEQ ID NO: 125, and/or wherein the anti-IL-18 antibody comprises a VL domain having an amino acid sequence that is identical to the full sequence of SEQ ID NO: 125; and/or wherein the anti-IL-18 antibody comprises an antibody VH domain and an antibody VL domain, wherein the amino acid sequence of the antibody VH domain is at least 90% identical to the full sequence of SEQ ID NO: 121, and the antibody VL domain is at least 90% identical to the full sequence of SEQ ID NO: 125.

21. (canceled)

22. (canceled)

23. (canceled)

24. (canceled)

25. The method of claim 1, wherein the anti-IL-18 antibody is administered in the form of a pharmaceutically acceptable composition.

26. The method of claim 1, wherein the anti-IL-18 antibody is administered for a period of at least 16 weeks, and/or wherein the anti-IL-18 antibody is administered once per week, once every two weeks, once every three weeks, once every four weeks, once every five weeks, or once every six weeks, and/or wherein the anti-IL-18 antibody is administered intravenously or subcutaneously.

27. (canceled)

28. (canceled)

29. (canceled)

30. The method of claim 1, wherein the subject achieves: resolution of fever, a reduction of CRP, a reduction of CRP of >50% from baseline to week 4, a reduction of CRP of >50% from baseline to week 12, a reduction of serum IL-18 levels, a reduction of serum total IL-18 levels, and/or a reduction of serum free IL-18 levels.

31. (canceled)

32. (canceled)

33. (canceled)

34. (canceled)

35. (canceled)

36. (canceled)

37. The method of claim 1, wherein the subject has: undetectable levels of serum IL-18 at about 4 weeks after administration of the anti-IL-18 antibody, undetectable levels of serum IL-18 at about 12 weeks after administration of the anti-IL-18 antibody, undetectable levels of serum total IL-18 at about 4 weeks after administration of the anti-IL-18 antibody, undetectable levels of serum total IL-18 at about 12 weeks after administration of the anti-IL-18 antibody, undetectable levels of serum free IL-18 at about 4 weeks after administration of the anti-IL-18 antibody, and/or undetectable levels of serum free IL-18 at about 12 weeks after administration of the anti-IL-18 antibody.

38. (canceled)

39. (canceled)

40. (canceled)

41. (canceled)

42. (canceled)

43. The method of claim 1, wherein the subject exhibits: a reduction of the signs and symptoms of AOSD or SoJIA as measured by the Physician and/or Patient Global Assessment of disease activity assessed on a Visual Analogue Scale (VAS), a reduction of the signs and symptoms of AOSD or SoJIA as measured by the Physician and/or Patient Global Assessment of disease activity assessed on a Visual Analogue Scale (VAS) at least at about 4 weeks after administration of the anti-IL-18 antibody, a reduction of the signs and symptoms of AOSD or SoJIA as measured by the Physician and/or Patient Global Assessment of disease activity assessed on a Visual Analogue Scale (VAS) at least at about 12 weeks after administration of the anti-IL-18 antibody, a reduction of the signs and symptoms of AOSD or SoJIA assessed using the modified Pouchot score, a reduction of the signs and symptoms of AOSD or SoJIA assessed using the modified Pouchot score at about 4 weeks after administration of the anti-IL-18 antibody, a reduction of the signs and symptoms of AOSD or SoJIA assessed using the modified Pouchot score at about 12 weeks after administration of the anti-IL-18 antibody, a reduction of the signs and symptoms of AOSD or SoJIA assessed using the DAS28-CRP score, a reduction of the signs and symptoms of AOSD or SoJIA assessed using the DAS28-CRP score at about 4 weeks after administration of the anti-IL-18 antibody, and/or a reduction of the signs and symptoms of AOSD or SoJIA assessed using the DAS28-CRP score at about 12 weeks after administration of the anti-IL-18 antibody.

44. (canceled)

45. (canceled)

46. (canceled)

47. (canceled)

48. (canceled)

49. (canceled)

50. (canceled)

51. (canceled)

52. The method of claim 1, wherein the subject experiences: a reduction in serum ferritin, a reduction in serum ferritin at about 4 weeks after administration of the anti-IL-18 antibody, a reduction in serum ferritin at about 12 weeks after administration of the anti-IL-18 antibody, a reduction in erythrocyte sedimentation rate (ESR), a reduction in erythrocyte sedimentation rate (ESR) at about 4 weeks after administration of the anti-IL-18 antibody, and/or a reduction in erythrocyte sedimentation rate (ESR) at about 12 weeks after administration of the anti-IL-18 antibody.

53. (canceled)

54. (canceled)

55. (canceled)

56. (canceled)

57. (canceled)

58. The method of claim 1, further comprising administering one or more additional therapeutically active agents, optionally wherein the one or more additional therapeutically active agents comprises at least one of non-steroidal anti-inflammatory drugs (NSAIDs), corticosteroids, systemic glucocorticoids, and conventional synthetic disease-modifying anti-rheumatic drugs (DMARDs).

59. (canceled)

60. The method of claim 58, wherein the conventional synthetic disease-modifying anti-rheumatic drug (DMARD) is methotrexate or wherein the corticosteroid is prednisone.

61. (canceled)

62. A kit for use in a method of claim 1, comprising an anti-IL-18 antibody and reagents for carrying out the method.


SEQUENCE LISTING (168 sequences)

SEQ ID NO: 1 - protein, 125 aa, synthetic construct
  REGION 1..125: note = Synthetic: Antibody 1 VH
QVQLQQSGPRLVKPSQTLSLTCTVSGGSISSGGYYWSWIRQPAGKGLEWIGSIYYSGSTYYNPSLKSRVTISGDTPKNQFSLKLSSVTAADTAVYYCARTPAYDGDARADFFDVWGRGTLVTVSS

SEQ ID NO: 2 - protein, 7 aa, synthetic construct
  REGION 1..7: note = Synthetic: Antibody 1 HCDR1
SGGYYWS

SEQ ID NO: 3 - protein, 16 aa, synthetic construct
  REGION 1..16: note = Synthetic: Antibody 1 HCDR2
SIYYSGSTYYNPSLKS

SEQ ID NO: 4 - protein, 15 aa, synthetic construct
  REGION 1..15: note = Synthetic: Antibody 1 HCDR3
TPAYDGDARADFFDV

SEQ ID NO: 5 - protein, 107 aa, synthetic construct
  REGION 1..107: note = Synthetic: Antibody 1 VL
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIK

SEQ ID NO: 6 - protein, 11 aa, synthetic construct
  REGION 1..11: note = Synthetic: Antibody 1 LCDR1
RASQGISSWLA

SEQ ID NO: 7 - protein, 7 aa, synthetic construct
  REGION 1..7: note = Synthetic: Antibody 1 LCDR2
KASTLES

SEQ ID NO: 8 - protein, 9 aa, synthetic construct
  REGION 1..9: note = Synthetic: Antibody 1 LCDR3
QQSYSTPWT

SEQ ID NO: 9 - protein, 125 aa, synthetic construct
  REGION 1..125: note = Synthetic: Antibody 1_GL VH
QVQLQESGPGLVKPSETLSLTCTVSGGSISSGGYYWSWIRQPPGKGLEWIGSIYYSGSTYYNPSLKSRVTISGDTSKNQFSLKLSSVTAADTAVYYCARTPAYDGDARADFFDVWGRGTLVTVSS

SEQ ID NO: 10 - protein, 7 aa, synthetic construct
  REGION 1..7: note = Synthetic: Antibody 1_GL HCDR1
SGGYYWS

SEQ ID NO: 11 - protein, 16 aa, synthetic construct
  REGION 1..16: note = Synthetic: Antibody 1_GL HCDR2
SIYYSGSTYYNPSLKS

SEQ ID NO: 12 - protein, 15 aa, synthetic construct
  REGION 1..15: note = Synthetic: Antibody 1_GL HCDR3
TPAYDGDARADFFDV

SEQ ID NO: 13 - protein, 107 aa, synthetic construct
  REGION 1..107: note = Synthetic: Antibody 1_GL VL
DIQMTQSPSTLSASVGDRVTITCRASQGISSWLAWYQQKPGKAPKVLIYKASTLESGVPSRFSGSGSGTEFTLTISSLQPDDFATYYCQQSYSTPWTFGQGTKLEIK

SEQ ID NO: 14 - protein, 11 aa, synthetic construct
  REGION 1..11: note = Synthetic: Antibody 1_GL LCDR1
RASQGISSWLA

SEQ ID NO: 15 - protein, 7 aa, synthetic construct
  REGION 1..7: note = Synthetic: Antibody 1_GL LCDR2
KASTLES

SEQ ID NO: 16 - protein, 9 aa, synthetic construct
  REGION 1..9: note = Synthetic: Antibody 1_GL LCDR3
QQSYSTPWT

SEQ ID NO: 17 - protein, 125 aa, synthetic construct; identical to SEQ ID NO: 1
  REGION 1..125: note = Synthetic: Antibody 2 VH

SEQ ID NO: 18 - protein, 7 aa, synthetic construct
  REGION 1..7: note = Synthetic: Antibody 2 HCDR1
SGGYYWS

SEQ ID NO: 19 - protein, 16 aa, synthetic construct
  REGION 1..16: note = Synthetic: Antibody 2 HCDR2
SIYYSGSTYYNPSLKS

SEQ ID NO: 20 - protein, 15 aa, synthetic construct
  REGION 1..15: note = Synthetic: Antibody 2 HCDR3
TPAYDGDARADFFDV

SEQ ID NO: 21 - protein, 107 aa, synthetic construct
  REGION 1..107: note = Synthetic: Antibody 2 VL
DIVMTQSPSTLSASVGDRVTITCRASQGISSWLAWYQQKPGRAPKVLIYKASTLESGVPSRFSGSGSGTDFTLTISSLQPEDFATYYCQDISFPPWTFGQGTKLEIK

SEQ ID NO: 22 - protein, 11 aa, synthetic construct
  REGION 1..11: note = Synthetic: Antibody 2 LCDR1
RASQGISSWLA

SEQ ID NO: 23 - protein, 7 aa, synthetic construct
  REGION 1..7: note = Synthetic: Antibody 2 LCDR2
KASTLES

SEQ ID NO: 24 - protein, 9 aa, synthetic construct
  REGION 1..9: note = Synthetic: Antibody 2 LCDR3
QDISFPPWT

SEQ ID NO: 25 - protein, 125 aa, synthetic construct; identical to SEQ ID NO: 1
  REGION 1..125: note = Synthetic: Antibody 3 VH

SEQ ID NO: 26 - protein, 7 aa, synthetic construct
  REGION 1..7: note = Synthetic: Antibody 3 HCDR1
SGGYYWS

SEQ ID NO: 27 - protein, 16 aa, synthetic construct
  REGION 1..16: note = Synthetic: Antibody 3 HCDR2
SIYYSGSTYYNPSLKS

SEQ ID NO: 28 - protein, 15 aa, synthetic construct
  REGION 1..15: note = Synthetic: Antibody 3 HCDR3
TPAYDGDARADFFDV

SEQ ID NO: 29 - protein, 107 aa, synthetic construct
  REGION 1..107: note = Synthetic: Antibody 3 VL
DIVMTQSPSTLSASVGDRVTITCRASQGISSWLAWYQQKPGRAPKVLIYKASTLESGVPSRFSGSGSGTDFTLTISSLQPEDFATYYCQQSLYPPWTFGQGTKLEIK

SEQ ID NO: 30 - protein, 11 aa, synthetic construct
  REGION 1..11: note = Synthetic: Antibody 3 LCDR1
RASQGISSWLA

SEQ ID NO: 31 - protein, 7 aa, synthetic construct
  REGION 1..7: note = Synthetic: Antibody 3 LCDR2
KASTLES

SEQ ID NO: 32 - protein, 9 aa, synthetic construct
  REGION 1..9: note = Synthetic: Antibody 3 LCDR3
QQSLYPPWT

SEQ ID NO: 33 - protein, 125 aa, synthetic construct; identical to SEQ ID NO: 1
  REGION 1..125: note = Synthetic: Antibody 4 VH

SEQ ID NO: 34 - protein, 7 aa, synthetic construct
  REGION 1..7: note = Synthetic: Antibody 4 HCDR1
SGGYYWS

SEQ ID NO: 35 - protein, 16 aa, synthetic construct
  REGION 1..16: note = Synthetic: Antibody 4 HCDR2
SIYYSGSTYYNPSLKS

SEQ ID NO: 36 - protein, 15 aa, synthetic construct
  REGION 1..15: note = Synthetic: Antibody 4 HCDR3
TPAYDGDARADFFDV

SEQ ID NO: 37 - protein, 107 aa, synthetic construct
  REGION 1..107: note = Synthetic: Antibody 4 VL
DIVMTQSPSTLSASVGDRVTITCRASQGISSWLAWYQQKPGRAPKVLIYKASTLESGVPSRFSGSGSGTDFTLTISSLQPEDFATYYCQQSHHPNWDFGQGTKLEIK

SEQ ID NO: 38 - protein, 11 aa, synthetic construct
  REGION 1..11: note = Synthetic: Antibody 4 LCDR1
RASQGISSWLA

SEQ ID NO: 39 - protein, 7 aa, synthetic construct
  REGION 1..7: note = Synthetic: Antibody 4 LCDR2
KASTLES

SEQ ID NO: 40 - protein, 9 aa, synthetic construct
  REGION 1..9: note = Synthetic: Antibody 4 LCDR3
QQSHHPNWD

SEQ ID NO: 41 - protein, 125 aa, synthetic construct; identical to SEQ ID NO: 1
  REGION 1..125: note = Synthetic: Antibody 5 VH

SEQ ID NO: 42 - protein, 7 aa, synthetic construct
  REGION 1..7: note = Synthetic: Antibody 5 HCDR1
SGGYYWS

SEQ ID NO: 43 - protein, 16 aa, synthetic construct
  REGION 1..16: note = Synthetic: Antibody 5 HCDR2
SIYYSGSTYYNPSLKS

SEQ ID NO: 44 - protein, 15 aa, synthetic construct
  REGION 1..15: note = Synthetic: Antibody 5 HCDR3
TPAYDGDARADFFDV

SEQ ID NO: 45 - protein, 107 aa, synthetic construct
  REGION 1..107: note = Synthetic: Antibody 5 VL
DIVMTQSPSTLSASVGDRVTITCRASQGISSWLAWYQQKPGRAPKVLIYKASTLESGVPSRFSGSGSGTDFTLTISSLQPEDFATYYCQQSLIPQWDFGQGTKLEIK

SEQ ID NO: 46 - protein, 11 aa, synthetic construct
  REGION 1..11: note = Synthetic: Antibody 5 LCDR1
RASQGISSWLA

SEQ ID NO: 47 - protein, 7 aa, synthetic construct
  REGION 1..7: note = Synthetic: Antibody 5 LCDR2
KASTLES

SEQ ID NO: 48 - protein, 9 aa, synthetic construct
  REGION 1..9: note = Synthetic: Antibody 5 LCDR3
QQSLIPQWD

SEQ ID NO: 49 - protein, 125 aa, synthetic construct
  REGION 1..125: note = Synthetic: Antibody 6 VH
QVQLQQSGPRLVKPSQTLSLTCTVSGGSISSGGYYWSWIRQPAGKGLEWIGSIYYSGSTYYNPSLKSRVTISGDTPKNQFSLKLSSVTAADTAVYYCARTPAYFGQDRTDFFDVWGRGTLVTVSS

SEQ ID NO: 50 - protein, 7 aa, synthetic construct
  REGION 1..7: note = Synthetic: Antibody 6 HCDR1
SGGYYWS

SEQ ID NO: 51 - protein, 16 aa, synthetic construct
  REGION 1..16: note = Synthetic: Antibody 6 HCDR2
SIYYSGSTYYNPSLKS

SEQ ID NO: 52 - protein, 15 aa, synthetic construct
  REGION 1..15: note = Synthetic: Antibody 6 HCDR3
TPAYFGQDRTDFFDV

SEQ ID NO: 53 - protein, 107 aa, synthetic construct
  REGION 1..107: note = Synthetic: Antibody 6 VL
DIVMTQSPSTLSASVGDRVTITCRASQGISSWLAWYQQKPGRAPKVLIYKASTLESGVPSRFSGSGSGTDFTLTISSLQPEDFATYYCANIAFPPWTFGQGTKLEIK

SEQ ID NO: 54 - protein, 11 aa, synthetic construct
  REGION 1..11: note = Synthetic: Antibody 6 LCDR1
RASQGISSWLA

SEQ ID NO: 55 - protein, 7 aa, synthetic construct
  REGION 1..7: note = Synthetic: Antibody 6 LCDR2
KASTLES

SEQ ID NO: 56 - protein, 9 aa, synthetic construct
  REGION 1..9: note = Synthetic: Antibody 6 LCDR3
ANIAFPPWT

SEQ ID NO: 57 - protein, 125 aa, synthetic construct
  REGION 1..125: note = Synthetic: Antibody 6_GL VH
QVQLQESGPGLVKPSETLSLTCTVSGGSISSGGYYWSWIRQPPGKGLEWIGSIYYSGSTYYNPSLKSRVTISGDTSKNQFSLKLSSVTAADTAVYYCARTPAYFGQDRTDFFDVWGRGTLVTVSS

SEQ ID NO: 58 - protein, 7 aa, synthetic construct
  REGION 1..7: note = Synthetic: Antibody 6_GL HCDR1
SGGYYWS

SEQ ID NO: 59 - protein, 16 aa, synthetic construct
  REGION 1..16: note = Synthetic: Antibody 6_GL HCDR2
SIYYSGSTYYNPSLKS

SEQ ID NO: 60 - protein, 15 aa, synthetic construct
  REGION 1..15: note = Synthetic: Antibody 6_GL HCDR3
TPAYFGQDRTDFFDV

SEQ ID NO: 61 - protein, 107 aa, synthetic construct
  REGION 1..107: note = Synthetic: Antibody 6_GL VL
DIQMTQSPSTLSASVGDRVTITCRASQGISSWLAWYQQKPGKAPKVLIYKASTLESGVPSRFSGSGSGTEFTLTISSLQPDDFATYYCANIAFPPWTFGQGTKLEIK

SEQ ID NO: 62 - protein, 11 aa, synthetic construct
  REGION 1..11: note = Synthetic: Antibody 6_GL LCDR1
RASQGISSWLA

SEQ ID NO: 63 - protein, 7 aa, synthetic construct
  REGION 1..7: note = Synthetic: Antibody 6_GL LCDR2
KASTLES

SEQ ID NO: 64 - protein, 9 aa, synthetic construct
  REGION 1..9: note = Synthetic: Antibody 6_GL
ANIAFPPWT

SEQ ID NO: 65 - protein, 125 aa, synthetic construct; identical to SEQ ID NO: 49
  REGION 1..125: note = Synthetic: Antibody 7 VH

SEQ ID NO: 66 - protein, 7 aa, synthetic construct
  REGION 1..7: note = Synthetic: Antibody 7 HCDR1
SGGYYWS

SEQ ID NO: 67 - protein, 16 aa, synthetic construct
  REGION 1..16: note = Synthetic: Antibody 7 HCDR2
SIYYSGSTYYNPSLKS

SEQ ID NO: 68 - protein, 15 aa, synthetic construct
  REGION 1..15: note = Synthetic: Antibody 7 HCDR3
TPAYFGQDRTDFFDV

SEQ ID NO: 69 - protein, 107 aa, synthetic construct
  REGION 1..107: note = Synthetic: Antibody 7 VL
DIVMTQSPSTLSASVGDRVTITCRASQGISSWLAWYQQKPGRAPKVLIYKASTLESGVPSRFSGSGSGTDFTLTISSLQPEDFATYYCQQSHHPPWTFGQGTKLEIK

SEQ ID NO: 70 - protein, 11 aa, synthetic construct
  REGION 1..11: note = Synthetic: Antibody 7 LCDR1
RASQGISSWLA

SEQ ID NO: 71 - protein, 7 aa, synthetic construct
  REGION 1..7: note = Synthetic: Antibody 7 LCDR2
KASTLES

SEQ ID NO: 72 - protein, 9 aa, synthetic construct
  REGION 1..9: note = Synthetic: Antibody 7 LCDR3
QQSHHPPWT

SEQ ID NO: 73 - protein, 125 aa, synthetic construct; identical to SEQ ID NO: 57
  REGION 1..125: note = Synthetic: Antibody 7_GL VH

SEQ ID NO: 74 - protein, 7 aa, synthetic construct
  REGION 1..7: note = Synthetic: Antibody 7_GL HCDR1
SGGYYWS

SEQ ID NO: 75 - protein, 16 aa, synthetic construct
  REGION 1..16: note = Synthetic: Antibody 7_GL HCDR2
SIYYSGSTYYNPSLKS

SEQ ID NO: 76 - protein, 15 aa, synthetic construct
  REGION 1..15: note = Synthetic: Antibody 7_GL HCDR3
TPAYFGQDRTDFFDV

SEQ ID NO: 77 - protein, 107 aa, synthetic construct
  REGION 1..107: note = Synthetic: Antibody 7_GL VL
DIQMTQSPSTLSASVGDRVTITCRASQGISSWLAWYQQKPGKAPKVLIYKASTLESGVPSRFSGSGSGTEFTLTISSLQPDDFATYYCQQSHHPPWTFGQGTKLEIK

SEQ ID NO: 78 - protein, 11 aa, synthetic construct
  REGION 1..11: note = Synthetic: Antibody 7_GL LCDR1
RASQGISSWLA

SEQ ID NO: 79 - protein, 7 aa, synthetic construct
  REGION 1..7: note = Synthetic: Antibody 7_GL LCDR2
KASTLES

SEQ ID NO: 80 - protein, 9 aa, synthetic construct
  REGION 1..9: note = Synthetic: Antibody 7_GL LCDR3
QQSHHPPWT

SEQ ID NO: 81 - protein, 125 aa, synthetic construct
  REGION 1..125: note = Synthetic: Antibody 8_GL VH
QVQLQESGPGLVKPSETLSLTCTVSGGSISAGGYYWSWIRQPPGKGLEWIGSLYYSGSTYYNPSLKGRVTISGDTSKNQFSLKLSSVTAADTAVYYCARTPAYFGQDRTDFFDVWGRGTLVTVSS

SEQ ID NO: 82 - protein, 7 aa, synthetic construct
  REGION 1..7: note = Synthetic: Antibody 8_GL HCDR1
AGGYYWS

SEQ ID NO: 83 - protein, 16 aa, synthetic construct
  REGION 1..16: note = Synthetic: Antibody 8_GL HCDR2
SLYYSGSTYYNPSLKG

SEQ ID NO: 84 - protein, 15 aa, synthetic construct
  REGION 1..15: note = Synthetic: Antibody 8_GL HCDR3
TPAYFGQDRTDFFDV

SEQ ID NO: 85 - protein, 107 aa, synthetic construct; identical to SEQ ID NO: 77
  REGION 1..107: note = Synthetic: Antibody 8_GL VL

SEQ ID NO: 86 - protein, 11 aa, synthetic construct
  REGION 1..11: note = Synthetic: Antibody 8_GL LCDR1
RASQGISSWLA

SEQ ID NO: 87 - protein, 7 aa, synthetic construct
  REGION 1..7: note = Synthetic: Antibody 8_GL LCDR2
KASTLES

SEQ ID NO: 88 - protein, 9 aa, synthetic construct
  REGION 1..9: note = Synthetic: Antibody 8_GL LCDR3
QQSHHPPWT

SEQ ID NO: 89 - protein, 125 aa, synthetic construct
  REGION 1..125: note = Synthetic: Antibody 9 VH
QVQLQQSGPRLVEPSQTLSLTCTVSGGSISSDGYYWSWIRQPAGKGLEWIGSIYYSGSTYYNPSLKSRVTISGDTPKNQFSLKLSSVTAADTAVYYCARTPAYFGQDRTDFFDVWGRGTLVTVSS

SEQ ID NO: 90 - protein, 7 aa, synthetic construct
  REGION 1..7: note = Synthetic: Antibody 9 HCDR1
SDGYYWS

SEQ ID NO: 91 - protein, 16 aa, synthetic construct
  REGION 1..16: note = Synthetic: Antibody 9 HCDR2
SIYYSGSTYYNPSLKS

SEQ ID NO: 92 - protein, 15 aa, synthetic construct
  REGION 1..15: note = Synthetic: Antibody 9 HCDR3
TPAYFGQDRTDFFDV

SEQ ID NO: 93 - protein, 107 aa, synthetic construct; identical to SEQ ID NO: 69
  REGION 1..107: note = Synthetic: Antibody 9 VL

SEQ ID NO: 94 - protein, 11 aa, synthetic construct
  REGION 1..11: note = Synthetic: Antibody 9 LCDR1
RASQGISSWLA

SEQ ID NO: 95 - protein, 7 aa, synthetic construct
  REGION 1..7: note = Synthetic: Antibody 9 LCDR2
KASTLES

SEQ ID NO: 96 - protein, 9 aa, synthetic construct
  REGION 1..9: note = Synthetic: Antibody 9 LCDR3
QQSHHPPWT

SEQ ID NO: 97 - protein, 125 aa, synthetic construct
  REGION 1..125: note = Synthetic: Antibody 10 VH
QVQLQQSGPRLVEPSQTLSLTCTVSGGSISSDGYYWSWIRQPAGKGLEWIGSIYYSGSTYYNPSLRSRVTISGDTPKNQFSLKLSSVTAADTAVYYCARTPAYFGQDRTDFFDVWGRGTLVTVSS

SEQ ID NO: 98 - protein, 7 aa, synthetic construct
  REGION 1..7: note = Synthetic: Antibody 10 HCDR1
SDGYYWS

SEQ ID NO: 99 - protein, 16 aa, synthetic construct
  REGION 1..16: note = Synthetic: Antibody 10 HCDR2
SIYYSGSTYYNPSLRS

SEQ ID NO: 100 - protein, 15 aa, synthetic construct
  REGION 1..15: note = Synthetic: Antibody 10 HCDR3
TPAYFGQDRTDFFDV

SEQ ID NO: 101 - protein, 107 aa, synthetic construct
  REGION 1..107: note = Synthetic: Antibody 10 VL
DIVMTQSPSTLSASVGDRVTITCRASQGISSWLAWYQQKPGGAPKVLIYKASTLESGVPSRFSGSGSGTDFTLTISSLQPEDFATYYCQQSHHPPWTFSQGTKLEIK

SEQ ID NO: 102 - protein, 11 aa, synthetic construct
  REGION 1..11: note = Synthetic: Antibody 10 LCDR1
RASQGISSWLA

SEQ ID NO: 103 - protein, 7 aa, synthetic construct
  REGION 1..7: note = Synthetic: Antibody 10 LCDR2
KASTLES

SEQ ID NO: 104 - protein, 9 aa, synthetic construct
  REGION 1..9: note = Synthetic: Antibody 10 LCDR3
QQSHHPPWT

SEQ ID NO: 105 - protein, 125 aa, synthetic construct
  REGION 1..125: note = Synthetic: Antibody 11 VH
QVQLQQSGPRLVEPSQTLSLTCTVSGGSISADGYYWSWIRQPAGKGLEWIGSLYYSGSTYYNPSLRGRVTISGDTPKNQFSLKLSSVTAADTAVYYCARTPAYFGQDRTDFFDVWGRGTLVTVSS

SEQ ID NO: 106 - protein, 7 aa, synthetic construct
  REGION 1..7: note = Synthetic: Antibody 11 HCDR1
ADGYYWS

SEQ ID NO: 107 - protein, 16 aa, synthetic construct
  REGION 1..16: note = Synthetic: Antibody 11 HCDR2
SLYYSGSTYYNPSLRG

SEQ ID NO: 108 - protein, 15 aa, synthetic construct
  REGION 1..15: note = Synthetic: Antibody 11 HCDR3
TPAYFGQDRTDFFDV

SEQ ID NO: 109 - protein, 107 aa, synthetic construct; identical to SEQ ID NO: 101
  REGION 1..107: note = Synthetic: Antibody 11 VL

SEQ ID NO: 110 - protein, 11 aa, synthetic construct
  REGION 1..11: note = Synthetic: Antibody 11 LCDR1
RASQGISSWLA

SEQ ID NO: 111 - protein, 7 aa, synthetic construct
  REGION 1..7: note = Synthetic: Antibody 11 LCDR2
KASTLES

SEQ ID NO: 112 - protein, 9 aa, synthetic construct
  REGION 1..9: note = Synthetic: Antibody 11 LCDR3
QQSHHPPWT

SEQ ID NO: 113 - protein, 125 aa, synthetic construct
  REGION 1..125: note = Synthetic: Antibody 11_GL VH
QVQLQESGPGLVKPSETLSLTCTVSGGSISADGYYWSWIRQPPGKGLEWIGSLYYSGSTYYNPSLRGRVTISGDTSKNQFSLKLSSVTAADTAVYYCARTPAYFGQDRTDFFDVWGRGTLVTVSS

SEQ ID NO: 114 - protein, 7 aa, synthetic construct
  REGION 1..7: note = Synthetic: Antibody 11_GL HCDR1
ADGYYWS

SEQ ID NO: 115 - protein, 16 aa, synthetic construct
  REGION 1..16: note = Synthetic: Antibody 11_GL HCDR2
SLYYSGSTYYNPSLRG

SEQ ID NO: 116 - protein, 15 aa, synthetic construct
  REGION 1..15: note = Synthetic: Antibody 11_GL HCDR3
TPAYFGQDRTDFFDV

SEQ ID NO: 117 - protein, 107 aa, synthetic construct; identical to SEQ ID NO: 77
  REGION 1..107: note = Synthetic: Antibody 11_GL VL

SEQ ID NO: 118 - protein, 11 aa, synthetic construct
  REGION 1..11: note = Synthetic: Antibody 11_GL LCDR1
RASQGISSWLA

SEQ ID NO: 119 - protein, 7 aa, synthetic construct
  REGION 1..7: note = Synthetic: Antibody 11_GL LCDR2
KASTLES

SEQ ID NO: 120 - protein, 9 aa, synthetic construct
  REGION 1..9: note = Synthetic: Antibody 11_GL LCDR3
QQSHHPPWT

SEQ ID NO: 121 - protein, 125 aa, synthetic construct
  REGION 1..125: note = Synthetic: Antibody 12_GL VH
QVQLQESGPGLVKPSETLSLTCTVSGGSISADGYYWSWIRQPPGKGLEWIGSLYYSGSTYYNPSLKGRVTISGDTSKNQFSLKLSSVTAADTAVYYCARTPAYFGQDRTDFFDVWGRGTLVTVSS

SEQ ID NO: 122 - protein, 7 aa, synthetic construct
  REGION 1..7: note = Synthetic: Antibody 12_GL HCDR1
ADGYYWS

SEQ ID NO: 123 - protein, 16 aa, synthetic construct
  REGION 1..16: note = Synthetic: Antibody 12_GL HCDR2
SLYYSGSTYYNPSLKG

SEQ ID NO: 124 - protein, 15 aa, synthetic construct
  REGION 1..15: note = Synthetic: Antibody 12_GL HCDR3
TPAYFGQDRTDFFDV

SEQ ID NO: 125 - protein, 107 aa, synthetic construct; identical to SEQ ID NO: 77
  REGION 1..107: note = Synthetic: Antibody 12_GL VL

SEQ ID NO: 126 - protein, 11 aa, synthetic construct
  REGION 1..11: note = Synthetic: Antibody 12_GL LCDR1
RASQGISSWLA

SEQ ID NO: 127 - protein, 7 aa, synthetic construct
  REGION 1..7: note = Synthetic: Antibody 12_GL LCDR2
KASTLES

SEQ ID NO: 128 - protein, 9 aa, synthetic construct
  REGION 1..9: note = Synthetic: Antibody 12_GL LCDR3
QQSHHPPWT

SEQ ID NO: 129 - protein, 5 aa, synthetic construct
  REGION 1..5: note = Synthetic: HCDR1
GYYFH

SEQ ID NO: 130 - protein, 17 aa, synthetic construct
  REGION 1..17: note = Synthetic: HCDR2
RIDPEDDSTKYAERFKD

SEQ ID NO: 131 - protein, 17 aa, synthetic construct
  REGION 1..17: note = Synthetic: HCDR3
WRIYRDSSGRPFYVMDA

SEQ ID NO: 132 - protein, 11 aa, synthetic construct
  REGION 1..11: note = Synthetic: LCDR1
LASEDIYTYLT

SEQ ID NO: 133 - protein, 7 aa, synthetic construct
  REGION 1..7: note = Synthetic: LCDR2
GANKLQD

SEQ ID NO: 134 - protein, 9 aa, synthetic construct
  REGION 1..9: note = Synthetic: LCDR3
LQGSKFPLT

SEQ ID NO: 135 - protein, 193 aa, Homo sapiens
  REGION 1..193: note = misc_feature - Human IL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 136 - DNA, 582 bp, Homo sapiens
  misc_feature 1..582: note = Human IL-18 (PN)
  source 1..582: mol_type = genomic DNA
atggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactag

SEQ ID NO: 137 - protein, 456 aa, synthetic construct
  REGION 1..456: note = Synthetic: H1 heavy chain
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - DNA, 1370 bp, synthetic construct
  misc_feature 1..1370: note = Synthetic: H1 heavy chain (PN)
  source 1..1370: mol_type = other DNA
aggtgcagctggtgcagagcggagccgaggtgaagaagcctggcgccagcgtcaaggtgtcctgtaaggtgtccggcgagatcagcaccggctactacttccactgggtgaggcaggcccctggcaagggcctggagtggatgggcagaatcgaccccgaggacgacagcaccaagtacgccgagcggttcaaggacagggtgaccatgaccgaggacaccagcaccgataccgcctacatggagctgtccagcctgagaagcgaggataccgccgtgtactactgtaccacctggcggatctacagagacagcagcggcagacccttctacgtgatggatgcctggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaagtga

SEQ ID NO: 139 - protein, 126 aa, synthetic construct
  REGION 1..126: note = Synthetic: H1 variable region
QVQLVQSGAEVKKPGASVKVSCKVSGEISTGYYFHWVRQAPGKGLEWMGRIDPEDDSTKYAERFKDRVTMTEDTSTDTAYMELSSLRSEDTAVYYCTTWRIYRDSSGRPFYVMDAWGQGTLVTVSS

SEQ ID NO: 140 - DNA, 378 bp, synthetic construct
  misc_feature 1..378: note = Synthetic: H1 variable region (PN)
  source 1..378: mol_type = other DNA
caggtgcagctggtgcagagcggagccgaggtgaagaagcctggcgccagcgtcaaggtgtcctgtaaggtgtccggcgagatcagcaccggctactacttccactgggtgaggcaggcccctggcaagggcctggagtggatgggcagaatcgaccccgaggacgacagcaccaagtacgccgagcggttcaaggacagggtgaccatgaccgaggacaccagcaccgataccgcctacatggagctgtccagcctgagaagcgaggataccgccgtgtactactgtaccacctggcggatctacagagacagcagcggcagacccttctacgtgatggatgcctggggccagggcacactagtgaccgtgtccagc

SEQ ID NO: 141 - protein, 214 aa, synthetic construct
  REGION 1..214: note = Synthetic: L2 light chain
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - DNA, 642 bp, synthetic construct
  misc_feature 1..642: note = Synthetic: L2 light chain (PN)
  source 1..642: mol_type = other DNA
gatatccagatgacccagtcccccagcagcgtgtccgcctctgtgggcgatagagtgaccatcacctgcctggccagcgaggacatctacacctacctgacctggtatcagcagaagcctggcaaggcccctaagctgctgatctacggcgccaacaagctgcaggacggcgtgcccagcagattcagcggcagcggctccggcaccgactacaccctgaccatcagcagcctgcagcctgaggatttcgccacctactactgcctgcagggcagcaagttccccctgaccttcggccagggcaccaagctggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 143 - protein, 107 aa, synthetic construct
  REGION 1..107: note = Synthetic: L2 variable region
DIQMTQSPSSVSASVGDRVTITCLASEDIYTYLTWYQQKPGKAPKLLIYGANKLQDGVPSRFSGSGSGTDYTLTISSLQPEDFATYYCLQGSKFPLTFGQGTKLEIK

SEQ ID NO: 144 - DNA, 321 bp, synthetic construct
  misc_feature 1..321: note = Synthetic: L2 variable region (PN)
  source 1..321: mol_type = other DNA
gatatccagatgacccagtcccccagcagcgtgtccgcctctgtgggcgatagagtgaccatcacctgcctggccagcgaggacatctacacctacctgacctggtatcagcagaagcctggcaaggcccctaagctgctgatctacggcgccaacaagctgcaggacggcgtgcccagcagattcagcggcagcggctccggcaccgactacaccctgaccatcagcagcctgcagcctgaggatttcgccacctactactgcctgcagggcagcaagttccccctgaccttcggccagggcaccaagctggagatcaag

SEQ ID NO: 145 - protein, 456 aa, synthetic construct
  REGION 1..456: note = Synthetic: H2 heavy chain
QVQLVQSGAEVKKPGASVKVSCKVSGEISTGYYFHWVRRRPGKGLEWMGRIDPEDDSTKYAERFKDRVTMTEDTSTDTAYMELSSLRSEDTAVYYCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - DNA, 1368 bp, synthetic construct
  misc_feature 1..1368: note = Synthetic: H2 heavy chain (PN)
  source 1..1368: mol_type = other DNA
caggtccagctggtacagtctggggctgaggtgaagaagcctggggcctcagtgaaggtctcctgcaaggtttccggagaaataagtactggatactatttccactgggtgcgacgaaggcctggaaaagggcttgagtggatgggaaggattgatcctgaggatgatagtactaaatatgctgagaggttcaaagacagagtcaccatgaccgaggacacatctacagacacagcctacatggagctgagcagcctgagatctgaggacacggccgtgtattactgtaccacatggcggatataccgagatagttctggccgccccttctatgttatggatgcctggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 147 - protein, 126 aa, synthetic construct
  REGION 1..126: note = Synthetic: H2 variable region
QVQLVQSGAEVKKPGASVKVSCKVSGEISTGYYFHWVRRRPGKGLEWMGRIDPEDDSTKYAERFKDRVTMTEDTSTDTAYMELSSLRSEDTAVYYCTTWRIYRDSSGRPFYVMDAWGQGTLVTVSS

SEQ ID NO: 148 - DNA, 378 bp, synthetic construct
  misc_feature 1..378: note = Synthetic: H2 variable region (PN)
  source 1..378: mol_type = other DNA
caggtccagctggtacagtctggggctgaggtgaagaagcctggggcctcagtgaaggtctcctgcaaggtttccggagaaataagtactggatactatttccactgggtgcgacgaaggcctggaaaagggcttgagtggatgggaaggattgatcctgaggatgatagtactaaatatgctgagaggttcaaagacagagtcaccatgaccgaggacacatctacagacacagcctacatggagctgagcagcctgagatctgaggacacggccgtgtattactgtaccacatggcggatataccgagatagttctggccgccccttctatgttatggatgcctggggccaagggacactagtcacagtctcctca

SEQ ID NO: 149 - protein, 456 aa, synthetic construct
  REGION 1..456: note = Synthetic: H3 heavy chain
QVQLVQSGAEVKKPGASVKVSCKVSGEISTGYYFHFVRRRPGKGLEWMGRIDPEDDSTKYAERFKDRVTMTADTSTDTAYMELSSLRSEDTATYFCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - DNA, 1368 bp, synthetic construct
  misc_feature 1..1368: note = Synthetic: H3 heavy chain (PN)
  source 1..1368: mol_type = other DNA
caggtccagctggtacagtctggggctgaggtgaagaagcctggggcctcagtgaaggtctcctgcaaggtttccggagaaataagtactggatactatttccactttgtgcgacgaaggcctggaaaagggcttgagtggatgggaaggattgatcctgaggatgatagtactaaatatgctgagaggttcaaagacagagtcaccatgaccgcagacacatctacagacacagcctacatggagctgagcagcctgagatctgaggacacggccacttatttttgtaccacatggcggatataccgagatagttctggccgccccttctatgttatggatgcctggggccaagggacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 151 - protein, 126 aa, synthetic construct
  REGION 1..126: note = Synthetic: H3 variable region
QVQLVQSGAEVKKPGASVKVSCKVSGEISTGYYFHFVRRRPGKGLEWMGRIDPEDDSTKYAERFKDRVTMTADTSTDTAYMELSSLRSEDTATYFCTTWRIYRDSSGRPFYVMDAWGQGTLVTVSS

SEQ ID NO: 152 - DNA, 378 bp, synthetic construct
  misc_feature 1..378: note = Synthetic: H3 variable region (PN)
  source 1..378: mol_type = other DNA
caggtccagctggtacagtctggggctgaggtgaagaagcctggggcctcagtgaaggtctcctgcaaggtttccggagaaataagtactggatactatttccactttgtgcgacgaaggcctggaaaagggcttgagtggatgggaaggattgatcctgaggatgatagtactaaatatgctgagaggttcaaagacagagtcaccatgaccgcagacacatctacagacacagcctacatggagctgagcagcctgagatctgaggacacggccacttatttttgtaccacatggcggatataccgagatagttctggccgccccttctatgttatggatgcctggggccaagggacactagtcacagtctcctca

SEQ ID NO: 153 - protein, 214 aa, synthetic construct
  REGION 1..214: note = Synthetic: L1 light chain
DIQMTQSPSSVSASVGDRVTITCLASEDIYTYLTWYQQKPGKAPKLLIYGANKLQDGVPSRFSGSGSGTDFTLTISSLQPEDFATYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - DNA, 642 bp, synthetic construct
  misc_feature 1..642: note = Synthetic: L1 light chain (PN)
  source 1..642: mol_type = other DNA
gacatccagatgacccagtctccatcttctgtgtctgcatctgtaggagacagagtcaccatcacttgtctggcaagtgaggacatatacacttatttaacatggtatcagcagaaaccagggaaagcccctaagctcctgatctatggtgcaaataagttgcaagatggggtcccatcaaggttcagcggcagtggatctgggacagatttcactctcactatcagcagcctgcagcctgaagattttgcaacttactattgtctacagggttccaagtttccgctcacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 155 - protein, 107 aa, synthetic construct
  REGION 1..107: note = Synthetic: L1 variable region
DIQMTQSPSSVSASVGDRVTITCLASEDIYTYLTWYQQKPGKAPKLLIYGANKLQDGVPSRFSGSGSGTDFTLTISSLQPEDFATYYCLQGSKFPLTFGQGTKLEIK

SEQ ID NO: 156 - DNA, 321 bp, synthetic construct
  misc_feature 1..321: note = Synthetic: L1 variable region (PN)
  source 1..321: mol_type = other DNA
gacatccagatgacccagtctccatcttctgtgtctgcatctgtaggagacagagtcaccatcacttgtctggcaagtgaggacatatacacttatttaacatggtatcagcagaaaccagggaaagcccctaagctcctgatctatggtgcaaataagttgcaagatggggtcccatcaaggttcagcggcagtggatctgggacagatttcactctcactatcagcagcctgcagcctgaagattttgcaacttactattgtctacagggttccaagtttccgctcacgtttggccaggggaccaagctggagatcaaa

SEQ ID NO: 157 - protein, 214 aa, synthetic construct
  REGION 1..214: note = Synthetic: L3 light chain
DIQMTQSPSSVSASVGDRVTITCLASEDIYTYLTWYQQKPGKAPQLLIYGANKLQDGVPSRFSGSGSGTDYTLTISSLQPEDEGDYYCLQGSKFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - DNA, 645 bp, synthetic construct
  misc_feature 1..645: note = Synthetic: L3 light chain (PN)
  source 1..645: mol_type = other DNA
gacatccagatgacccagtctccatcttctgtgtctgcatctgtaggagacagagtcaccatcacttgtctggcaagtgaggacatatacacttatttaacatggtatcagcagaaaccagggaaagcccctcaactcctgatctatggtgcaaataagttgcaagatggggtcccatcaaggttcagcggcagtggatctgggacagattatactctcactatcagcagcctgcagcctgaagatgaaggggattactattgtctacagggttccaagtttccgctcacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 159 - protein, 107 aa, synthetic construct
  REGION 1..107: note = Synthetic: L3 variable region
DIQMTQSPSSVSASVGDRVTITCLASEDIYTYLTWYQQKPGKAPQLLIYGANKLQDGVPSRFSGSGSGTDYTLTISSLQPEDEGDYYCLQGSKFPLTFGQGTKLEIK

SEQ ID NO: 160 - DNA, 321 bp, synthetic construct
  misc_feature 1..321: note = Synthetic: L3 variable region (PN)
  source 1..321: mol_type = other DNA
gacatccagatgacccagtctccatcttctgtgtctgcatctgtaggagacagagtcaccatcacttgtctggcaagtgaggacatatacacttatttaacatggtatcagcagaaaccagggaaagcccctcaactcctgatctatggtgcaaataagttgcaagatggggtcccatcaaggttcagcggcagtggatctgggacagattatactctcactatcagcagcctgcagcctgaagatgaaggggattactattgtctacagggttccaagtttccgctcacgtttggccaggggaccaagctggagatcaaa

SEQ ID NO: 161 - protein, 456 aa, synthetic construct
  REGION 1..456: note = Synthetic: 2c10 rat-human IgG1 chimera
EVQLQQSGAELVRPGTSVKLSCKVSGEISTGYYFHFVRRRPGQGLEWIGRIDPEDDSTKYAERFKDRATLTAQTSSNTAYLNLSSLTSEDTATYFCTTWRIYRDSSGRPFYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - DNA, 1368 bp, synthetic construct
  misc_feature 1..1368: note = Synthetic: 2c10 rat-human IgG1 chimera (PN)
  source 1..1368: mol_type = other DNA
gaggtccagctacagcagtctggggctgagcttgtgagacctgggacctctgtgaagttatcttgcaaagtttctggcgaaataagtacaggatactatttccactttgtgaggcgaaggcctggacagggtctggaatggataggaaggattgatcctgaggatgatagtactaaatatgctgagaggttcaaagacagggcgacgctcactgcacaaacatcctccaacacagcctacctgaacctcagcagcctgacctctgaggacactgcaacttatttttgtaccacatggcggatataccgagatagttctggccgccccttctatgttatggatgcctggggtcaaggaacactagtcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 163 - protein, 214 aa, synthetic construct
  REGION 1..214: note = Synthetic: 2c10 rat-human CKappa chimera
DIQMTQSPASLSASLGETVSIECLASEDIYTYLTWYQQKPGKSPQLLIYGANKLQDGVPSRFSGSGSGTQYSLKISGIQPEDEGDYFCLQGSKFPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - DNA, 642 bp, synthetic construct
  misc_feature 1..642: note = Synthetic: 2c10 rat-human CKappa chimera (PN)
  source 1..642: mol_type = other DNA
gacattcaaatgacccagtctccagcttccctgtctgcatctctgggagaaactgtctccatcgaatgtctggcaagtgaggacatatacacttatttaacatggtatcagcagaaaccagggaaatctcctcaactcctgatctatggtgcaaataagttgcaagatggggtcccatcacggttcagtggcagtggatctggcacacagtattctctcaagatcagcggcatacaacctgaagatgaaggggattatttctgtctacagggttccaagtttccgctcacgttcggttctgggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggacaacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 165 - protein, 98 aa, synthetic construct
  REGION 1..98: note = Synthetic: Heavy chain acceptor framework
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 166 - protein, 95 aa, synthetic construct
  REGION 1..95: note = Synthetic: Light chain acceptor framework
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 167 - protein, 11 aa, synthetic construct
  REGION 1..11: note = Synthetic: JH6 amino acid sequence added to SEQ ID NO: 37
WGQGTLVTVSS

SEQ ID NO: 168 - protein, 10 aa, synthetic construct
  REGION 1..10: note = Synthetic: Jkappa 2 amino acid sequence added to SEQ ID NO: 38
FGQGTKLEIK